(19) United States
(12) Patent Application Publication
(10) Pub. No.: US 2003/0207327 A1
(43) Pub. Date: Nov. 6, 2003

(54) Coisogenic eukaryotic cell collections

(76) Inventors: Eric B. Kmiec, Landenberg, PA (US); Michael C. Rice, Newton, PA (US)

Correspondence Address: FISH & NEAVE, 1251 AVENUE OF THE AMERICAS, 50TH FLOOR, NEW YORK, NY, 10020-1105, US

(21) Appl. No.: 10/260,638

(22) Filed: Sep. 27, 2002

Related U.S. Application Data
(60) Provisional application No. 60/325,992, filed on Sep. 27, 2001.

(51) Int. Cl. G01N 33/53; C12N 1/18; C12N 5/10; C12N 5/06; C12N 5/08

(52) U.S. Cl. 435/7.1; 435/354; 435/366; 435/254.2

(57) ABSTRACT

Collections of cultured eukaryotic cells, particularly human cells, in which the cells are coisogenic at a common target locus, are provided. Particularly provided are collections of coisogenic cells that differ in genomic sequence by no more than 0.05%, excluding changes at the target locus, collections in which the coisogenic cells differ in genomic sequence by no more than 0.005%, excluding changes at the target locus, and collections in which the cells lack heterologous genetic elements within 10 kilobases of the coisogenic target locus. Kits comprising the cell collections, methods of making the collections, kits for making the collections, and methods of using the collections to facilitate pharmacogenomic analyses are presented. Preferred target loci at which the cells are coisogenic include genes that affect drug resistance, drug sensitivity, and/or drug metabolism.

### CROSS-REFERENCE TO RELATED APPLICATIONS

This application claims the benefit of U.S. provisional application serial No. 60/325,992, filed Sep. 27, 2001, the disclosure of which is incorporated herein by reference in its entirety.

### FIELD OF THE INVENTION

The present invention is in the field of molecular biology, and relates to coisogenic eukaryotic cell collections and methods of use therefor. More specifically, the invention relates to collections of eukaryotic cells that have been engineered to differ from one another by as few as one encoded amino acid at a defined target locus, particularly, but not exclusively, target loci that encode proteins that affect responsiveness to therapeutic agents, and to pharmacogenomic methods based thereupon.

### BACKGROUND OF THE INVENTION

The newly-emerging field of pharmacogenomics is premised on the notion that statistical correlations of genotypic variations that occur naturally within a population (allelic variation) with their respective phenotypes can be used to predict an individual patient's responsiveness to therapy based upon knowledge of the patient's genotype; the ultimate goal is to stratify patient populations into genetic cohorts for which therapy can be separately tailored. See, e.g., Adam et al., “Pharmacogenomics to predict drug response,” Pharmacogenomics 1 (1):5-14 (2000); Judson et al., “The predictive power of haplotypes in clinical response,” Pharmacogenomics 1 (1):15-26 (2000).

As a preliminary to any such clinical prognostication, naturally occurring alleles must be identified and the alleles correlated with observable clinical phenotypes. A sufficient number of individuals must be studied for the correlations to achieve statistical reliability. Each of these requirements limits the utility of current pharmacogenomic approaches.

Although the first of these limitations is being addressed, in part, by public, quasi-public and private undertakings to identify all common single nucleotide polymorphisms (SNPs) in the human genome (see, e.g., NCBI's dbSNP database at http://www.ncbi.nlm.nih.gov/SNP/; the Karolinska Institute's Human Genic Bi-Allelic Sequences Database at http://hgbase.cgr.ki.se/; and the SNP Consortium's database at http://snp.cshl.org/), patients carrying uncommon, perhaps unique, alleles will remain outside the prognostic scope of such analyses. Furthermore, the requirement for observable clinical phenotypes and the requirement for patient populations of adequate statistical size are not addressed by the simple expedient of cataloguing common SNPs.

One clinical phenotype that has been proposed for pharmacogenomic-based prognostication is multidrug resistance. See, e.g., Kerb et al., “ABC drug transporters: hereditary polymorphisms and pharmacological impact in MDR1, MRP1 and MRP2,” Pharmacogenomics 2(1):51-64 (2000); Szakacs et al., “Diagnostics of multidrug resistance in cancer,” Pathol. Oncol. Res. 4(4):251-7 (1998).

Genetic polymorphisms in proteins other than the multidrug transporters are also known to play a role in drug sensitivity and in drug resistance. For example, the cytochrome 450 enzyme encoded by CYP2D6 is known to metabolize as many as 20% of commonly prescribed drugs. The gene is highly polymorphic in the population; certain alleles result in the poor metabolizer phenotype, characterized by a decreased ability to metabolize the enzyme's substrates.

In vitro assays have been developed to assess the drug sensitivity of individual cells. For example, U.S. Pat. Nos. 6,277,655 and 5,872,014 describe assays specific for activity of the multidrug transporter ABCB1 (MDR1), as does Ludescher et al., Br. J. Haematol. 82(1):161-8 (1992). See also, “In vitro assays for chemotherapy sensitivity,” Crit. Rev. Oncol. Hematol. 15(2):99-111 (1993); Cree et al., “Tumor chemosensitivity and chemoresistance assays,” Cancer 78(9):2031-2 (1996); Apoptosis and Cell Proliferation, 2^{nd} ed., Boehringer Mannheim, 1998 (available on-line at http://biochem.boehringer-mannheim.com/prod_inf/manuals/cell_man/acp.pdf), and Poirier (ed.), Apoptosis Techniques and Protocols, Humana Press, 1997 (ISBN: 0896034518).

Although the in vitro drug resistance (equally and conversely, drug sensitivity) phenotype of individual cells can at times predict the clinical phenotype of the entire organism, to apply such in vitro assays to pharmacogenomic analyses requires the in vitro assay of cells bearing different alleles of the gene or genes of interest. Few such alleles are available in cell lines that can readily be assayed, and when available, are often present on genetically disparate backgrounds.

Recently, there have been efforts to create collections of cell lines that have defined genetic modifications on a uniform genetic background for use in various in vitro assays.

Genetic modifications that have typically been contemplated for eukaryotic cells used in screening assays include targeted deletion or disruption of genes, dominant negative suppression of gene expression, and change in gene copy number. See, e.g., U.S. Pat. Nos. 5,569,588, 5,777,888, 6,165,709, 6,046,002. For the most part, the preferred organism for such genetic modification has been yeast, notably Saccharomyces cerevisiae, due in part to its ability to support homologous recombination at efficiencies far greater than those possible in mammalian cells. Where the cell line is mammalian, however, often the chosen modification leaves heterologous nucleic acids at or near the target locus, a legacy of virally-mediated modification events. See, e.g., U.S. Pat. No. 6,207,371.

Thus, there exists a need for methods that would more readily permit pharmacogenomic analyses without requiring the prior large scale correlation of naturally-occurring alleles with naturally-occurring, clinically observable phenotypes. There is a further need in the art for collections of eukaryotic cells, particularly mammalian cells, that have defined mutations in target loci, particularly mutations that recapitulate naturally-occurring alleles, on a uniform genetic background. There is a particular need for collections of eukaryotic cells that lack heterologous nucleic acid insertions additional to the targeted changes. In particular, there exists a need for such cell collections having targeted mutations in genes that affect drug resistance.

### SUMMARY OF THE INVENTION

The present invention satisfies these and other objects in the art by providing, in a first aspect a collection of cultured cells, comprising at least 5, 10, or at least 25 genotypically distinct cells, wherein each of the genotypically distinct cells is coisogenic with respect to the others in the collection at a common target locus. The genotypically distinct cells of the collection are separately assayable.

As used herein, two genotypically distinct cells are “coisogenic” with respect to one another if derived from a common ancestor cell and engineered to differ from one another in genomic sequence at a predetermined target locus. The genomic sequence differences at the target locus must be sufficient to alter the amino acid sequence encoded at the target locus by at least one amino acid. The term “coisogenic” permits of changes as between the genomes of the genotypically distinct cells additional to the changes at the target locus.

In certain preferred embodiments, the coisogenic cells of the collection are “exceptionally coisogenic”, that is, differ in genomic sequence by no more than 0.05%, excluding changes at the target locus, or “perfectly coisogenic”, differing in genomic sequence by no more than 0.005%, excluding changes at the target locus. In certain preferred embodiments, the cells are alternatively, or additionally, legacy-free, that is, lacking in heterologous genetic elements within 10 kilobases of any codon of the target locus.

The coisogenic cells can be from any eukaryote; although usefully mammalian, especially human, the cells can also be of yeast or plant origin.

In certain embodiments, the genotypically distinct cells of the collection collectively include each of the 20 natural amino acids at a single residue encoded at the target locus. In other embodiments, the genotypically distinct cells collectively include a predetermined amino acid at each residue encoded after the initiator methionine at the target locus. In particularly preferred embodiments, the genotypically distinct cells collectively include at least one, and on occasion a plurality, of naturally occurring allele of the target locus.

The cells of the collection can further comprise a common selectable marker at a genomic locus different from said target locus, and/or a marker unique to said genotypically distinct cell, the unique marker being at a locus different from the target locus.

The target locus can be any locus of interest, and in particularly useful embodiments, is selected from the group of loci affecting drug resistance (sensitivity) or drug metabolism consisting of: CYP1A2, CYP2C17, CYP2D6, CYP2E, CYP3A4, CYP4A11, CYP1B1, CYP1A1, CYP2A6, CYP2A13, CYP2B6, CYP2C8, CYP2C9, CYP11A, CYP2C19, CYP2F1, CYP2J2, CYP3A5, CYP3A7, CYP4B1, CYP4F2, CYP4F3, CYP6D1, CYP6F1, CYP7A1, CYP8, CYP11A, CYP11B1, CYP11B2, CYP17, CYP19, CYP21A2, CYP24, CYP27Al, CYP51, ABCB1, ABCB4, ABCC1, ABCC2, ABCC3, ABCC4, ABCC5, ABCC6, MRP7, ABCC8, ABCC9, ABCC10, ABCC11, ABCC12, EPHX1, EPHX2, LTA4H, TRAG3, GUSB, TMPT, BCRP, HERG, hKCNE2, UDP glucuronosyl transferase (UGT), sulfotransferase, sulfatase, glutathione S-transferase (GST)-alpha, glutathione S-transferase-mu, glutathione S-transferase-pi, ACE, and KCHN2.

In another aspect, the invention provides the coisogenic cell collection in the form of a kit. The kit comprises at least five genotypically distinct cells, the cells contained within separate, structurally discrete, fluidly noncommunicating containers, wherein each of the genotypically distinct cells is coisogenic with respect the others at a target locus common thereamong; the structurally discrete containers are commonly packaged.

In some embodiments, the kit further comprises a computer readable medium, recorded upon which is a dataset (typically, a relational database) that describes the target locus genotype of each of said genotypically distinct cells.

In another aspect, the invention provides a method of making a coisogenic cell collection.

In its most basic form, the method comprises collecting at least 5 genotypically distinct cells, each of the genotypically distinct cells being coisogenic with respect to the others at a target locus common thereamong, into a collection in which each of the genotypically distinct cells can be separately assayed.

Typically, the coisogenic cells will first be prepared, and the method will thus further comprise the antecedent step of engineering, into at least four of five cultured cells, the cells having derived from a common eukaryotic ancestor cell, a genomic sequence alteration at a target locus common thereamong. For purposes of the present invention, the sequence alterations should be sufficient to cause at least five distinct protein sequences collectively to be encoded by the cells at the target locus.

In preferred embodiments, the engineering is effected by introducing a targeting oligonucleotide into each of said at least four cultured cells. The targeting oligonucleotide effects site-specific change to the cellular genomic DNA. Alternatively, in a multistep process, a targeting oligonucleotide is first used to effect a change in a genomic recombination-competent substrate, such as an artificial chromosome, and the recombination-competent substrate then introduced into each of the four cultured cells.

In another aspect, the invention provides a kit useful for creating the coisogenic cell collections of the present invention. The kit comprises at least four targeting oligonucleotides of distinct sequence; and a eukaryotic cell. The targeting oligonucleotides are sufficient to effect four different sequence changes, each sequence change sufficient to alter the protein sequence, at the target genomic locus.

The coisogenic cell collections of the present invention can be used for multiplex, including high throughput multiplex screening for mutations that affect a cellular phenotype in vitro.

Thus, in another aspect, the invention provides a method of identifying genotypes of a target locus that alter a cellular phenotype, comprising a first step of assaying each genotypically distinct cell of a coisogenic cell collection for a common phenotypic characteristic. The genotypically distinct cells are coisogenic at the target locus, preferably exceptionally or perfectly coisogenic, and/or legacy-free. After assay, the method calls for identifying from the assay results at least one cell having an altered phenotypic characteristic; and correlating, for the cell or cells with altered phenotypic characteristic, the results of said phenotypic assay with the cell's target locus genotype. Such correlation of phenotypic assay results with target locus genotype identifies genotypes of the target locus that alter the cellular phenotype.

Usefully, the phenotypic characteristic can be responsiveness of the cell to a xenobiotic, and the method can thus include the antecedent step of contacting the coisogenic cell collection with a xenobiotic. In certain embodiments of the method, the cells of the collection are coisogenic at a target selected from the group consisting of: CYP1A2, CYP2C17, CYP2D6, CYP2E, CYP3A4, CYP4A11, CYP1B1, CYP1A1, CYP2A6, CYP2A13, CYP2B6, CYP2C8, CYP2C9, CYP11A, CYP2C19, CYP2F1, CYP2J2, CYP3A5, CYP3A7, CYP4B1, CYP4F2, CYP4F3, CYP6D1, CYP6Fl, CYP7A1, CYP8, CYP11A, CYP11B1, CYP11B2, CYP17, CYP19, CYP21A2, CYP24, CYP27A1, CYP51, ABCB1, ABCB4, ABCC1, ABCC2, ABCC3, ABCC4, ABCC5, ABCC6, MRP7, ABCC8, ABCC9, ABCC10, ABCC11, ABCC12, EPHX1, EPHX2, LTA4H, TRAG3, GUSB, TMPT, BCRP, HERG, hKCNE2, UDP glucuronosyl transferase (UGT), sulfotransferase, sulfatase, glutathione S-transferase (GST)-alpha, glutathione S-transferase-mu, glutathione S-transferase-pi, ACE, and KCHN2.

The correlations can thereafter optionally be collected into at least one dataset, typically one or more relational databases, usefully recorded on a computer-readable medium.

In a further aspect, the invention provides a method of predicting a phenotypic characteristic of a cell based upon its genotype at a target locus. The method comprises using the cell's genotype at the target locus, or a unique identifier thereof, as a query to retrieve from a dataset data that report a correlated phenotypic characteristic, wherein the dataset includes such correlations for at least five cells that are coisogenic at the target locus; the retrieved phenotypic characteristic provides a prediction of the cell's phenotypic characteristic.

The above and other objects and advantages of the present invention will be apparent upon consideration of the following detailed description.

### DETAILED DESCRIPTION OF THE INVENTION

Definitions

Unless otherwise made explicitly clear by context, the indefinite article “a” intends one or more of the objects referenced immediately thereafter.

As used herein, the term “cell” intends a eukaryotic cell. Unless otherwise made explicitly clear by context, the singular term “cell” equally intends a plurality of genetically identical cells, such as a plurality of cells from a clonal eukaryotic cell line. A “cultured cell” is a eukaryotic cell (or clonal eukaryotic cell line) that is maintained alive in vitro in nutrient media, or that has previously been propagated in vitro in nutrient media for at least one doubling.

“Genotypically distinct” cells have nonidentical genomic sequences.

A “target locus” is a genomic region that includes all exons of an expressed protein.

As used herein, two genotypically distinct cells are “coisogenic” with respect to one another if derived from a common ancestor cell and engineered to differ from one another in genomic sequence at a predetermined target locus. The genomic sequence differences at the target locus must be sufficient to alter the amino acid sequence encoded at the target locus by at least one amino acid. The term “coisogenic” permits of changes as between the genomes of the genotypically distinct cells additional to the changes at the target locus.

“Exceptionally coisogenic” cells are coisogenic cells that differ in genomic sequence by no more than 0.05%, excluding changes at the target locus.

“Perfectly coisogenic” cells are coisogenic cells that differ in genomic sequence by no more than 0.005%, excluding changes at the target locus.

Cells, or genetic alterations, therein are said to be “legacy-free” if lacking in heterologous genetic elements within 10 kilobases of an engineered genomic sequence alteration. When used with respect to coisogenic cells, the cells are legacy-free if lacking in heterologous genetic elements within 10 kilobases of any codon of the target locus.

As used herein, “heterologous genetic elements” are sequences of greater than 25 consecutive nucleotides that derive from—and that can thus be shown to be present in—species different from that from which the coisogenic cells derive; heterologous genetic elements thus include, inter alia, all genetic elements derived from prokaryotic cells, including prokaryotic genomic DNA; genetic elements derived from prokaryotic episomes, including fertility factors; genetic elements derived from bacteriophage; as well as genetic elements from eukaryotic viruses.

As used herein, the term “collection”, as applied to cells, intends that the cells are in sufficient spatial proximity to one another as readily and contemporaneously to be subject to the same experimental protocol. The term “library” is intended to be synonymous with “collection” in all respects.

As used herein, the term “xenobiotic” intends a foreign compound introduced into a biological system, such as an inorganic or organic compound foreign to the cell or organism under study, or a compound naturally present in the cell or organism under study but administered by normatural routes or at unnatural concentrations.

Coisogenic Eukaryotic Cell Collections, Methods of Making, and Methods of Use

The present invention is made possible by our recent discovery of methods and compositions, to be described in further detail below, for creating site-specific mutations in genomic DNA of eukaryotic cells, including mammalian cells, at efficiencies and with a precision not hitherto achievable using homologous recombination or earlier approaches based upon oligonucleotide-mediated gene repair.

The methods permit point mutations to be targeted with high efficiency to genomic DNA incubated in cellular extracts, such as artificial chromosomes incubated in cellular extracts, and also permit mutations to be targeted with high efficiency directly into the chromosomes of cultured cells. The efficiency is sufficiently high as to obviate the concomitant insertion of selectable markers or other exogenous DNA, permitting cells with defined mutations to be created legacy-free. These methods permit us readily to create collections of coisogenic eukaryotic cell lines, including legacy-free, perfectly coisogenic cell lines, that possess targeted and discrete changes at given target loci.

These collections of coisogenic cells have substantial utility in pharmacogenomic studies, obviating the identification of naturally-occurring allelic variants, observation of naturally occurring clinically-relevant phenotypes in a human population, and association of the naturally-occurring allelic variants with the naturally-occurring, clinically-relevant phenotypes. In embodiments particularly useful for pharmacogenomic studies, the target loci at which the collection of cells are coisogenic encode proteins known to affect drug resistance (conversely, drug sensitivity), and drug metabolism.

The collections of coisogenic cells have further utility in studies of the structure-activity relationships of existing, and of potential new, therapeutic agents, permitting multiplex analysis of the effects of amino acid changes on ligand-receptor interactions. The collections of coisogenic cells are also useful in screening for agonists and antagonists of proteins that affect drug resistance, sensitivity, and metabolism.

Thus, in a first aspect, the invention provides a collection of at least 5 genotypically distinct cells, typically as a collection of at least 5 genotypically distinct eukaryotic cell lines. Each of the genotypically distinct cells (or cell lines) is coisogenic to the others of the genotypically distinct cells (or cell lines) in the collection at a common target locus. In addition, each of the genotypically distinct cells can be separately assayed.

Given the generality of our oligonucleotide-mediated mutational approach, the cultured cells of the invention can be any eukaryotic cell amenable to in vitro culture.

Among mammalian cells, human cells have particular utility, particularly for pharmacogenomic uses. Also very useful, particularly for structure-activity studies, are cells from related primates, such as chimpanzee, monkeys (including rhesus macaque), baboon, orangutan, and gorilla, and those from rodents typically used as laboratory models, such as rats, mice, hamsters and guinea pigs. Cells can also usefully be from lagomorphs, such as rabbits; and from larger mammals, such as livestock, including horses, cattle, sheep, pigs, goats, and bison. Also useful are cells from fowl such as chickens, geese, ducks, turkeys, pheasant, ostrich and pigeon; fish such as zebrafish, salmon, tilapia, catfish, trout and bass; and domestic pet species, such as dogs and cats.

Plant cells for which coisogenic cell collections can usefully be constructed according to the methods of the present invention include, for example, experimental model plants, such as Chlamydomonas reinhardtii, Physcomitrella patens, and Arabidopsis thaliana; crop plants such as cauliflower (Brassica oleracea), artichoke (Cynara scolymus); fruits such as apples (Malus, e.g. Malus domesticus), mangoes (Mangifera, e.g. Mangifera indica), banana (Musa, e.g. Musa acuminata), berries (such as currant, Ribes, e.g. rubrum), kiwifruit (Actinidia, e.g. chinensis), grapes (Vitis, e.g. vinifera), bell peppers (Capsicum, e.g. Capsicum annuum), cherries (such as the sweet cherry, Prunus, e.g. avium), cucumber (Cucumis, e.g. sativus), melons (Cucumis, e.g. melo), nuts (such as walnut, Juglans, e.g. regia; peanut, Arachis hypogeae), orange (Citrus, e.g. maxima), peach (Prunus, e.g. Prunus persica), pear (Pyra, e.g. communis), plum (Prunus, e.g. domestica), strawberry (Fragaria, e.g. moschata or vesca), tomato (Lycopersicon, e.g. esculentum); leaves and forage, such as alfalfa (Medicago, e.g. sativa or truncatula), cabbage (e.g. Brassica oleracea), endive (Cichoreum, e.g. endivia), leek (Allium, e.g. porrum), lettuce (Lactuca, e.g. sativa), spinach (Spinacia, e.g. oleraceae), tobacco (Nicotiana, e.g. tabacum); roots, such as arrowroot (Maranta, e.g. arundinacea), beet (Beta, e.g. vulgaris), carrot (Daucus, e.g. carota), cassava (Manihot, e.g. esculenta), turnip (Brassica, e.g. rapa), radish (Raphanus, e.g. sativus), yam (Dioscorea, e.g. esculenta), sweet potato (Ipomoea batatas); seeds, including oilseeds, such as beans (Phaseolus, e.g. vulgaris), pea (Pisum, e.g. sativum), soybean (Glycine, e.g. max), cowpea (Vigna unguiculata), mothbean (Vigna aconitifolia), wheat (Triticum, e.g. aestivum), sorghum (Sorghum e.g. bicolor), barley (Hordeum, e.g. vulgare), corn (Zea, e.g. mays), rice (Oryza, e.g. sativa), rapeseed (Brassica napus), millet (Panicum sp.), sunflower (Helianthus annuus), oats (Avena sativa), chickpea (Cicer, e.g. arietinum); tubers, such as kohlrabi (Brassica, e.g. oleraceae), potato (Solanum, e.g. tuberosum) and the like; fiber and wood plants, such as flax (Linum, e.g. Linum usitatissimum), cotton (Gossypium e.g. hirsutum), pine (Pinus spp.), oak (Quercus sp.), eucalyptus (Eucalyptus sp.), and the like; and ornamental plants such as turfgrass (Lolium, e.g. rigidum), petunia (Petunia, e.g. x hybrida), hyacinth (Hyacinthus orientalis), carnation (Dianthus e.g. caryophyllus), delphinium (Delphinium, e.g. ajacis), Job's tears (Coix lacryma-jobi), snapdragon (Antirrhinum majus), poppy (Papaver, e.g. nudicaule), lilac (Syringa, e.g. vulgaris), hydrangea (Hydrangea e.g. macrophylla), roses (including Gallicas, Albas, Damasks, Damask Perpetuals, Centifolias, Chinas, Teas and Hybrid Teas) and ornamental goldenrods (e.g. Solidago spp.).

Given the conservation of basic metabolic pathways among all eukaryotes, cell collections of the present invention can also usefully be drawn from lower eukaryotes, such as yeasts, particularly Saccharomyces cerevisiae, Schizosaccharomyces pombe, Pichia species, such as methanolica, Ustillago maydis, and Candida species, from roundworms, such as C. elegans, from zebra fish, and from Drosophila melanogaster.

Eukaryotic cell lines from which coisogenic collections of the present invention may be created are readily available from a wide variety of sources known in the art, including the American Type Culture Collection (Manassas, Va., USA), the Deutsche Sammlung von Mikroorganismen und Zellkulturen (DSMZ, German Collection of Microorganisms and Cell Cultures), and the Riken Cell bank of Japan; 472 such culture collections are listed at http://wdcm.nig.ac.jp/hpcc.html.

Specialized cell collections are also well known, and include the NIGMS (National Institute of General Medical Studies) Human Genetic Cell Repository, the NIA Aging Cell Repository, the Autism Research Resource, the ADA Cell Repository Maturity Onset Diabetes Collection, and the HBDI Cell Repository Juvenile Diabetes Collection, all of which are maintained at the Coriell Institute for Medical Studies (Camden, N.J., USA). Specialized yeast collections include the National Collection of Yeast Cultures (Institute of Food Research, Norwich Research Park, Colney, Norwich, UK).

Existing cell lines are also amply well described in the literature. See, e.g., Drexler, The Leukemia-Lymphoma Cell Line FactsBook, (ISBN: 0122219708) (2000); Hay et al. (eds.), Atlas of Human Tumor Cell Lines, Academic Press, 1994 (ISBN: 0123335302); Masters et al. (eds.), Human Cell Culture: Cancer Cell Lines: Leukemias and Lymphomas, Vol. 3, Kluwer Academic, 2000 (ISBN: 079236225X); Dix (ed.), Plant Cell Line Selection: Procedures and Applications, John Wiley and Sons, 1990 (ISBN: 3527279636); Panchal (ed.), Yeast Strain Selection, Marcel Dekker, 1990 (ISBN: 0824782763).

Furthermore, methods are well known in the art for creating immortalized cell lines from a wide variety of primary cells having advantageous characteristics. For recent reviews see, e.g., Yeager et al., “Constructing immortalized human cell lines,” Curr. Opin. Biotechnol. 10(5):465-9 (1999); Rhim, “Development of human cell lines from multiple organs,” Ann. NY Acad. Sci. 919:16-25 (2000); McLean, “Improved techniques for immortalizing animal cells,” Trends Biotechnol. 11(6):232-8 (1993); and Hopfer et al., “Immortalization of epithelial cells,” Am. J. Physiol. 270(1 Pt 1):C1-C11 (1996).

Although at times preferred for convenience, the genotypically distinct cells need not be immortalized, or otherwise capable of indefinite propagation.

The collection includes at least 5 coisogenic cells (typically, as clonal cell lines). Higher assay throughput is often obtained when the collection includes greater than 5, such as 6, 7, 8, 9, or 10 genotypically distinct, coisogenic cells. Collections of 24 coisogenic cells can conveniently be disposed in a 24 well culture plate; collections of 96 coisogenic cells can conveniently be arrayed in a 96 well microtiter dish. With recent development of microtiter dishes with footprint identical to that of the standard microtiter dish, but with higher well density, collections of 384, 864, 1536, 3456, 6144, and as many as 9600 coisogenic cells can readily and usefully be present in the cell collections of the present invention. The collections need not necessarily contain such even numbers of genotypically distinct exceptionally coisogenic cells, and can thus include any number of genotypically distinct coisogenic cells greater than or equal to 5, including 6, 7, 8, 9, 10, 11, 12, 13, 14, 15, 16, 17, 18, 19, 20, 30, 40, 50, 60, 70, 75, 80, 85, 90, 95, 100, 200, 300, 400, 500 or more.

At least five of the genotypically distinct cells of the collections of the present invention are coisogenic at a common, predetermined, target locus. The target locus can be any protein-encoding locus of the cell. As will be further described below, preferred targets for pharmacogenomic studies encode proteins known to be involved in drug resistance and/or drug metabolism.

As defined herein, coisogenic cells have genomic sequence differences at the target locus that are sufficient to occasion change of at least one amino acid at the target locus. The genotypically distinct cells of the collection are coisogenic to the others of the genotypically distinct cells of the collection.

The methods and compositions for creating the coisogenic cells, which are further described below, readily permit the legacy-free substitution, addition, or deletion of as few as 1 and as many as 3 consecutive nucleotides in the genomic DNA of the target locus.

Alterations can include, for example, substitutions of one, two or three contiguous nucleotides, thus effecting a change in the amino acid encoded by one codon or by two adjacent codons. Since the standard genetic code is well known, the nucleotide changes required to effect change from any given codon to one that encodes any other desired amino acid would be apparent to the skilled artisan; examples are also presented herein below.

In one such embodiment, one predetermined amino acid residue is commonly targeted for change in each of the coisogenic cells; with a minimum of 20 genotypically distinct cells in the collection, each of the commonly occurring natural amino acids can be present in the collection at the target residue. Residues that are particularly informative as targets are those that occur in the protein at locations of known structural and/or functional importance, such as within highly structured, ligand-binding domains.

In an alternative embodiment, the genotypically distinct cells can differ not at the identical residue, but at successive amino acids of the target protein. By way of example, each genotypically distinct cell can contain a single alanine substitution. Thus, without disturbing the initiator methionine, the first cell of the collection can have alanine substituted for residue 2; the second cell of the collection can have alanine substituted for residue 3; the third cell of the collection can have alanine substituted for residue 4, etc. Collectively, the coisogenic cells of the cell collection present an in vivo alanine scan of the entire protein sequence, permitting ready identification of critical residues of the target protein.

Any amino acid can be used as the substitute in such an embodiment, with the choice dictated by the known chemical and biological properties of the naturally occurring amino acids. For example, proline can be substituted to effect disruption of secondary structures, such as beta sheets or alpha helices; tyrosine can be substituted to provide substrates for tyrosine-kinase mediated post-translational modification; glutamic acid can be substituted to increase local charge density.

Alterations can also include introduction of a termination codon. Because any codon of the target locus can be targeted, coisogenic cells can be collected that each individually possess a single engineered termination codon, but that collectively present consecutive, single amino acid truncations from the carboxy terminus of the target protein.

Alterations can also include insertion of an amino acid, through targeted insertion of a novel codon between two existing codons.

Alterations can, in other embodiments, include frameshift mutations, caused by insertion or deletion of 1 or 2 nucleotides. Frameshift can lead to truncation or elongation, depending upon presence of termination codons in the new reading frame. Introduction of compensating frameshifts (e.g., insertion of a single nucleotide followed, at some distance downstream, by deletion of a single nucleotide), can lead to alteration of a series of amino acids between the mutated nucleotides.

Other types of changes that can be created by targeted point mutations will be readily apparent to one skilled in the art.

Among the changes that can usefully be made, and that have particular utility for pharmacogenomic studies, are those that recapitulate naturally-occurring allelic variants at the target locus; such changes permit the phenotype occasioned by a naturally occurring alleles to be assessed against a common, defined, genetic background.

As would be understood, highly multiplex analyses can be done by combining the mutational embodiments set forth above. For example, the collection can include cells that are coisogenic at a first residue of the target locus, with the collection including all possible amino acids at that first target residue, with the collection further including cells that have substitutions at other residues of the target locus.

Greater differences can be achieved by targeting changes iteratively to the target locus using the methods of the present invention.

Furthermore, changes can be introduced into both alleles of the target locus, either in a single step or by iterative modification, thus creating a homozygous change. At present, homozygous changes are most desired, although heterozygous changes are permitted.

In certain embodiments, the coisogenic cells are legacy-free.

In certain embodiments, our methods for constructing coisogenic cell collections, further described below, can alter genomic DNA without concomitant insertion of heterologous nucleic acids, such as selectable markers, prokaryotic genetic elements, bacteriophage genetic elements, or eukaryotic viral elements, at the target locus. Because such heterologous nucleic acid close to the target locus can cause unpredictable changes in expression and/or activity of the target protein, they are disfavored, although permitted, in certain embodiments of the cell collections of the present invention.

Depending on their distance from a common cellular ancestor, the coisogenic cells of the present invention will, on occasion, have accumulated genetic differences at other than the target locus. Such differences are permissible.

In certain particularly useful embodiments, however, the coisogenic cells of the collections of the present invention, are “exceptionally coisogenic”, differing in genomic sequence by no more than 0.05%, excluding changes at the target locus. In other embodiments, the cells are “perfectly coisogenic”, differing in genomic sequence by no more than about 0.005%, excluding changes at the target locus. The exceptionally coisogenic cell collections and perfectly coisogenic cell collections of the present invention can each, additionally, be legacy-free.

The coisogenic cells of the cell collections of the present invention can also include intentional genetic changes at locations in the genome other than the target locus.

For example, mutations can be targeted to a second target locus, creating cell lines that are coisogenic at several targets.

As another example, markers, including selectable markers, can usefully, but optionally, be included, at a site other than the target locus. Such marker can be common to all cells in the collection, for example by prior introduction into a cellular ancestor common to all of the genotypically distinct cells, can be unique to each genotype, or can be common to some, but not to all, genotypically distinct cells in the collection.

For example, a selectable marker can commonly be included in all of the genotypically distinct cells of the collection to prevent overgrowth, either by cells of the same lineage, or by other species. Selectable markers are well known, and the choice thereof will depend upon the species from which the genotypically distinct cells of the collection are derived. Selectable markers for use in mammalian cells, e.g., include markers that confer resistance to neomycin (G418), blasticidin, hygromycin or to zeocin; other well-known selections are based upon the purine salvage pathway. Selectable markers in yeast include a variety of auxotrophic markers, such as alleles of URA3, HIS3, LEU2, TRP1 and LYS2.

At the other end of the spectrum, unique markers can be introduced into each of the genotypically distinct cells of the collection, allowing each genotypically distinct cell (typically, cell line) in the collection readily to be distinguished.

For example, the sequence can encode substrate-independent proteinaceous fluorophores with distinct emission spectra. See, e.g., Palm et al., “Spectral Variants of Green Fluorescent Protein,” in Green Fluorescent Proteins, Conn (ed.), Methods Enzymol. vol. 302, pp. 378-394 (1999)), the disclosure of which is incorporated herein by reference.

The markers can also be intended to distinguish the cells at the nucleic acid, rather than protein, level (genetic “bar codes”). If such bar codes are flanked by priming sites that are common to all of the bar codes of distinct sequence, a single amplification reaction (e.g., by PCR), can be used to stoichiometrically to amplify all bar codes, the presence and/or frequencies of which can thereafter readily be assayed. See, e.g., U.S. Pat. No. 6,046,002.

Other genetic alterations that can usefully be made outside the target locus include those that facilitate assay of the cells of the coisogenic cell collection of the present invention, as will be discussed below.

The target locus for the coisogenic cell collections of the present invention can be any locus believed to contribute to a relevant cellular or organismic phenotype, and thus usefully includes all proteins that are presently subject to drug screening assays (e.g., G protein coupled receptors, protein kinases, zinc finger-containing transcription factors), or pharmacogenomic analysis (such as ApoE, presenilin 1, presenilin 2, p53, etc.). Particularly useful targets in certain embodiments of the present invention are loci that encode proteins that affect drug responsiveness, in part because the clinical phenotype can readily be correlated with a cellular phenotype, permitting ready assay in vitro.

Accordingly, the cell collections of the present invention can usefully be coisogenic at loci that encode any one of the P450 enzymes, which are known significantly to affect the metabolism of many, if not most, therapeutic agents.

The cytochrome P450 superfamily includes a large number (as many as 60 in human beings) of separate, but related, monooxygenases that play a central role in oxidative metabolism of a wide range of compounds, including therapeutic drugs. Although the number of known P450 enzymes is large, and the endogenous substrates of most unknown, a half dozen or so appear to be responsible for metabolism of the vast majority of prescribed and over-the-counter drugs: CYP1A2, CYP2C17, CYP2D6, CYP2E (“CYP2E1”), CYP3A4, and CYP4A11. For recent reviews, see Anzenbacher et al., “Cytochromes P450 and metabolism of xenobiotics,” Cell. Mol. Life Sci. 58(5-6):737-47 (2001), and Drug. Ther. Bull. 38(12):93-5 (2000).

The cell collections of the present invention can thus usefully be coisogenic at CYP1A2 (cytochrome P450, subfamily I (aromatic compound-inducible), polypeptide 2) (also known as CP12, P3-450, P450(PA)). This gene, the human homologue of which is located about 25 kb away from CYP1A1 on chromosome 15 (at 15q22-qter), encodes a member of the cytochrome P450 superfamily of enzymes closely related to CYP1A1. The gene is aromatic compound-inducible, and is known to metabolize acetaminophen in human beings to the cytotoxic metabolite N-acetylbenzoquinoneimine (NABQI), Thatcher et al., Cancer Gene Ther. 7(4):521-5 (2000).

CYP2C17 can also usefully be targeted.

CYP2D6 (also known as CPD6, CYP2D, CYP2D@, P450C2D, P450-DB1) encodes cytochrome P450, subfamily IID (debrisoquine, sparteine, etc., -metabolizing), polypeptide 6, and is known to metabolize as many as 20% of commonly prescribed drugs; the cell collections of the present invention can usefully be coisogenic at this locus.

The enzyme's substrates include debrisoquine, an adrenergic-blocking drug; sparteine and propafenone, both anti-arrhythmic drugs; and amitryptiline, an anti-depressant. The gene is highly polymorphic in the population; certain alleles result in the poor metabolizer phenotype, characterized by a decreased ability to metabolize the enzyme's substrates. The gene is located near two cytochrome P450 pseudogenes on chromosome 22q13.1.

CYP2E (earlier denominated CPE1, CYP2E1, P450-J, P450C2E) encodes cytochrome P450, subfamily IIE (ethanol-inducible), located in the human genome at 10q24.3-qter, and can usefully be targeted in constructing coisogenic cell collections of the present invention. This P450 enzyme localizes to the endoplasmic reticulum and is induced by ethanol, the diabetic state, and starvation. The enzyme metabolizes both endogenous substrates, such as ethanol, acetone, and acetal, as well as exogenous substrates including benzene, carbon tetrachloride, ethylene glycol, and nitrosamines which are premutagens found in cigarette smoke. Due to its many substrates, this enzyme may be involved in such varied processes as gluconeogenesis, hepatic cirrhosis, diabetes, and cancer.

Another locus at which the cell collections of the present invention can usefully be coisogenic is CYP3A4 (also known as CP34, NF-25, P450C3, P450PCN1), which encodes cytochrome P450, subfamily IIIA (nifedipine oxidase), polypeptide 4.

The enzyme encoded by CYP3A4 localizes to the endoplasmic reticulum and its expression is induced by glucocorticoids and some pharmacological agents. This enzyme is involved in the metabolism of approximately half the drugs used today, including nifedipine, acetaminophen, codeine, cyclosporin A, diazepam and erythromycin. The enzyme also metabolizes some steroids and carcinogens.

Vinca alkaloids are important chemotherapeutic agents, and their pharmacokinetic properties display significant interindividual variations, possibly due to CYP3A4-mediated metabolism. See, Yao et al., “Detoxication of vinca alkaloids by human P450 CYP3A4-mediated metabolism: implications for the development of drug resistance,” J. Pharmacol. Exp. Ther. 294(1):387-95 (2000).

This gene is part of a cluster of cytochrome P450 genes on chromosome 7q21.1. Previously, another CYP3A gene, CYP3A3, was thought to exist; however, it is now thought that this sequence represents a transcript variant of CYP3A4.

CYP4A11 (also called CP4Y, CYP4A2, CYP4A11), encodes cytochrome P450, subfamily IVA, polypeptide 11, and can usefully serve as a target locus for the coisogenic cell collections of the present invention. CYP4A11 encodes a member of the cytochrome P450 superfamily of enzymes. This protein localizes to the endoplasmic reticulum and hydroxylates medium-chain fatty acids such as laurate and myristate.

Other cytochrome P450 enzymes can also usefully be targeted.

CYP1B1 (synonyms: CP1B, GLC3A), another target at which the cell collections of the present invention can usefully be coisogenic, encodes cytochrome P450, subfamily I (dioxin-inducible), polypeptide 1 (glaucoma 3, primary infantile), located in the human genome at 2p21. The P450 monooxygenase encoded by this gene localizes to the endoplasmic reticulum and metabolizes procarcinogens such as polycyclic aromatic hydrocarbons and 17beta-estradiol. Mutations in this gene have been associated with primary congenital glaucoma; therefore it is thought that the enzyme also metabolizes a signaling molecule involved in eye development, possibly a steroid.

Expression of CYP1B1, as with expression of CYP1A1, has been shown to be increased in an anti-estrogen-resistant breast cell line, Brockdorff et al., Int. J. Cancer 88(6):902-6 (2000), and has been generally implicated in tumor drug resistance, Rochat et al., “Human CYP1B1 and anticancer agent metabolism: mechanism for tumor-specific drug inactivation?”, J. Pharmacol. Exp. Ther. 296(2):537-41 (2001); McFadyen et al., “Cytochrome P450 CYP1B1 protein expression: a novel mechanism of anticancer drug resistance,” Biochem Pharmacol. 62(2):207-12 (2001).

CYP1A1 (cytochrome P450, subfamily I (aromatic compound-inducible), polypeptide1) (also known as AHH, AHRR, CP11, CYP1, P1-450, P450-C, P450DX), the human homologue of which is located at 15q22-24, can also usefully be targeted. Expression and activity of CYP1A are known to be induced by some polycyclic aromatic hydrocarbons (PAHs), some of which are found in cigarette smoke, and the enzyme is able to metabolize some PAHs to carcinogenic intermediates; the gene has specifically been associated with lung cancer risk.

CYP1A activity has been shown to be increased in a breast cell line resistant to the antiestrogen compound ICI 1827801, Brockdorff et al., “Increased expression of cytochrome p450 1A1 and 1B1 genes in anti-estrogen-resistant human breast cancer cell lines,” Int. J; Cancer 88(6):902-6 (2000), and has been suggested as a marker for sensitivity to anti-cancer drugs, Peters et al., “A mutation in exon 7 of the human cytochrome P-4501A1 gene as marker for sensitivity to anti-cancer drugs?”, Br. J. Cancer 75(9):1397 (1997).

Another target for which cell collections of the present invention can usefully be coisogenic is CYP2A6, the human homologue of which is found at 19q13.2, encoding cytochrome P450, subfamily IIA (phenobarbital-inducible), polypeptide 6 (also known as CPA6, CYP2A3). CYP2A6 encodes a P450 enzyme that localizes to the endoplasmic reticulum; its expression is induced by phenobarbital. The enzyme is known to hydroxylate coumarin, and also metabolizes nicotine, aflatoxin B1, nitrosamines, and some pharmaceuticals.

Individuals with certain allelic variants of CYP2A6 are said to have a “poor metabolizer” phenotype, meaning they do not efficiently metabolize drugs that are substantially metabolized by CYP2A6, such as coumarin, nicotine, or fluoxetine (Prozac®). CYP2A6 is part of a large cluster of cytochrome P450 genes from the CYP2A, CYP2B and CYP2F subfamilies on chromosome 19q.

CYP2A6 is predominantly responsible for the metabolism of nicotine to cotinine, and many allelic variants have been described. See, Zabetian et al., “Functional variants at CYP2A6: new genotyping methods, population genetics, and relevance to studies of tobacco dependence,” Am. J. Med. Genet. 96(5):638-45 (2000).

Another cytochrome P450 enzyme that can usefully be targeted in the coisogenic cell collections of the present invention is CYP2A13 (also known as CPAD), the human homologue of which is located at 19q13.2. CYP2A13 is phenobarbital-inducible, and is highly active in the metabolic activation of a major tobacco-specific carcinogen, 4-(methylnitrosamino)-1-(3-pyridyl)-1-butanone, with a catalytic efficiency much greater than that of other human cytochrome P450 isoforms. Su et al., “Human cytochrome P450 CYP2A13: predominant expression in the respiratory tract and its high efficiency metabolic activation of a tobacco-specific carcinogen, 4-(methylnitrosamino)-1-(3-pyridyl)-1-butanone,” Cancer Res 60(18):5074-9 (2000).

CYP2B6 (alternatively denominated CPB6, IIB1, P450, and CYPIIB6), encoding cytochrome P450, subfamily IIA (phenobarbital-inducible), polypeptide 6, is located at 19q13.2 in the human genome, and is a useful target locus for the coisogenic cell collections of the present invention. This P450 enzyme localizes to the endoplasmic reticulum and its expression is induced by phenobarbital. The enzyme is known to metabolize some xenobiotics, such as the anti-cancer drugs cyclophosphamide and ifosphamide. Transcript variants for this gene have been described; however, it has not been resolved whether these transcripts are in fact produced by this gene or by a closely related pseudogene, CYP2B7. Both the gene and the pseudogene are located in the middle of a CYP2A pseudogene found in a large cluster of cytochrome P450 genes from the CYP2A, CYP2B and CYP2F subfamilies on chromosome 19q. CYP2B6 is though to mediate the N-demethylation of (R)- and (S)-ketamine in human liver.

CYP2C8 (same as CPC8, P450 MP-12/MP-20) encoding cytochrome P450, subfamily IIC (mephenytoin 4-hydroxylase), polypeptide 8, is also a useful target for the coisogenic eukaryotic cell collections of the present invention. This protein localizes to the endoplasmic reticulum and its expression is induced by phenobarbital. The enzyme is known to metabolize many xenobiotics, including the anticonvulsive drug mephenytoin, benzo(a)pyrene, 7-ethyoxycoumarin, and the anti-cancer drug paclitaxel (Taxol®). CYP2C8 also metabolizes cerivastatin, which is a high potency, third generation synthetic statin with proven lipid-lowering efficacy.

Two transcript variants for this gene have been described; it is thought that the longer form does not encode an active cytochrome P450 since its protein product lacks the heme binding site. This gene is located within a cluster of cytochrome P450 genes on chromosome 10q24.

Another useful target for the coisogenic cell collections of the present invention is CYP2C9 (cytochrome P450, subfamily IIC (mephenytoin 4-hydroxylase), polypeptide 9), whose expression is induced by rifampin, and which is known to metabolize many xenobiotics, including phenytoin, tolbutamide, ibuprofen, aspirin and S-warfarin. See, e.g., Bigler et al., “CYP2C9 and UGT1A6 genotypes modulate the protective effect of aspirin on colon adenoma risk,” Cancer Res. 61(9):3566-9 (2001).

Studies identifying individuals who are poor metabolizers of phenytoin and tolbutamide suggest that this gene is polymorphic. The gene is located within a cluster of cytochrome P450 genes on chromosome 10q24.

CYP11A (same as P450SCC, cytochrome P450C11A1), also usefully targeted in the coisogenic cell collections of the present invention, encodes a member of the cytochrome P450 superfamily of enzymes. This protein localizes to the mitochondrial inner membrane and catalyzes the conversion of cholesterol to pregnenolone, the first and rate-limiting step in the synthesis of the steroid hormones. The human homologue is located at 15q23-q24.

CYP2C19 (same as CPCJ, CYP2C, P450C2C, P450IIC19, microsomal monooxygenase, xenobiotic monooxygenase, mephenytoin 4′-hydroxylase, flavoprotein-linked monooxygenase), encodes cytochrome P450, subfamily IIC (mephenytoin 4-hydroxylase), polypeptide 19. This protein localizes to the endoplasmic reticulum and is known to metabolize many xenobiotics, including the anticonvulsive drug mephenytoin, omeprazole, diazepam, proguanil, and some barbiturates. The enzyme is also responsible for the polymorphic (NAT2*) acetylation of hydrazine and aromatic amine drugs, such as isoniazid, hydralazine, and sulfasalazine. Polymorphism within this gene is associated with variable ability to metabolize mephenytoin, known respectively as the poor metabolizer phenotype and extensive metabolizer phenotype. The gene is located within a cluster of cytochrome P450 genes on chromosome 10q24, at 10q24.1-q24.3.

Other cytochrome P450 enzymes that can usefully be targeted to create the coisogenic cell collections of the present invention include CYP2F1, CYP2J2, CYP3A5, CYP3A7 (catalyzes the prenatal 4-hydroxylation of retinoic acid, playing an important role in protecting the human fetus against retinoic acid-induced embryotoxicity, Chen et al., “Catalysis of the 4-hydroxylation of retinoic acids by cyp3a7 in human fetal hepatic tissues,” Drug. Metab. Dispos. 28(9):1051-7 (2000)), CYP4B1, CYP4F2 (found to catalyze hydroxylation and dealkylation of an H(1)-antihistamine prodrug, ebastine, Hashizume et al., “A novel cytochrome p450 enzyme responsible for the metabolism of ebastine in monkey small intestine,” Drug Metab. Dispos. 29(6):798-805 (2001)), CYP4F3, CYP6Dl, CYP6F1 (related to CYP6D1 and involved in pyrethroid detoxification in insects), CYP7A1, CYP8, CYP11A, CYP11B1, CYP11B2, CYP17, CYP19, CYP21A2, CYP24, CYP27A1, and CYP51.

Other loci that affect drug resistance are also useful targets for oligonucleotide-mediated alterations for creating eukaryotic coisogenic cell collections of the present invention.

Among such non-P450 loci are the genes encoding ATP-binding cassette (ABC) proteins, which transport various molecules across extra- and intra-cellular membranes. ABC genes are divided into seven distinct subfamilies (ABC1, MDR/TAP, MRP, ALD, OABP, GCN20, White); some members are well known to confer a multi-drug (multiple drug) resistance phenotype on tumor cells.

Best known among the ABC proteins is ABCB1 (ATP-binding cassette, sub-family B (MDR/TAP), member 1), known alternatively as MDR1 (multi drug resistance 1), P-GP (P-glycoprotein), PGY1, ABC20, and GP170, the human homologue of which maps to 7q21.1.

The protein encoded by this gene is an ATP-dependent drug efflux pump for xenobiotic compounds with broad substrate specificity. It is responsible for decreased drug accumulation in multidrug-resistant cells and often mediates the development of resistance to anticancer drugs. A number of studies have demonstrated a negative correlation between Pgp expression levels and chemosensitivity or survival in a range of human malignancies. Lehne, “P-glycoprotein as a drug target in the treatment of multidrug resistant cancer,” Curr. Drug Targets 1(1):85-99 (2000).

P-glycoprotein is also expressed in normal tissues with excretory function such as liver, kidney and intestine. Apical expression of P-glycoprotein in such tissues results in reduced drug absorption from the gastrointestinal tract and enhanced drug elimination into bile and urine. Moreover, expression of P-glycoprotein in the endothelial cells of the blood-brain barrier prevents entry of certain drugs into the central nervous system. Human P-glycoprotein has been shown to transport a wide range of structurally unrelated drugs such as digoxin, quinidine, cyclosporin and HIV-1 protease inhibitors. Studies in humans indicate a particular importance of intestinal P-glycoprotein for bioavailability of the immunosuppressant cyclosporin. Moreover, induction of intestinal P-glycoprotein by rifampin has now been identified as the major underlying mechanism of reduced digoxin plasma concentrations during concomitant rifampin therapy. For reviews, see Fromm, “P-glycoprotein: a defense mechanism limiting oral bioavailability and CNS accumulation of drugs,” Int. J. Clin. Pharmacol. Ther. 38(2):69-74 (2000); Schinkel, “P-Glycoprotein, a gatekeeper in the blood-brain barrier,” Adv. Drug Deliv. Rev. 36(2-3):179-194 (1999); Van Asperen et al., “The pharmacological role of P-glycoprotein in the intestinal epithelium,” Pharmacol Res. 37(6):429-35 (1998); Tanigawara, “Role of P-glycoprotein in drug disposition,” Ther. Drug Monit. 22(1):137-40 (2000); and Schinkel, “The physiological function of drug-transporting P-glycoproteins,” Semin. Cancer Biol. 8(3):161-70 (1997).

Allelic variants of ABCB1 (MDR1) are known to affect its selectivity and/or activity. Hoffmeyer et al., “Functional polymorphisms of the human multidrug-resistance gene: multiple sequence variations and correlation of one allele with P-glycoprotein expression and activity in vivo,” Proc. Natl. Acad. Sci USA 97(7):3473-8 (2000); Choi et al., “An altered pattern of cross-resistance in multidrug-resistant human cells results from spontaneous mutations in the mdr1 (P-glycoprotein) gene,” Cell 53(4):519-29 (1988).

ABCB4 (ATP-binding cassette, sub-family B (MDR/TAP), member 4)(also known as MDR3, PGY3, ABC21, MDR2/3, PFIC-3) (human homologue maps to 7q21.1), is another useful target locus for the coisogenic cell collections of the present invention.

The membrane-associated protein encoded by this gene is a member of the superfamily of ATP-binding cassette (ABC) transporters. ABCB4 is a member of the MDR/TAP subfamily. Members of the MDR/TAP subfamily are involved in multidrug resistance as well as antigen presentation. This gene encodes a full transporter and member of the p-glycoprotein family of membrane proteins with phosphatidylcholine as its substrate.

ABCC1—ATP-binding cassette, sub-family C (CFTR/MRP), member 1—(same as MRP, ABCC, GS-X, MRP1, ABC29) is a member of the MRP subfamily of ATP-binding cassette (ABC) proteins, and is involved in multi-drug resistance. This protein functions as a multispecific organic anion transporter, with oxidized glutathione, cysteinyl leukotrienes, and activated aflatoxin B1 as known substrates. This protein also transports glucuronides and sulfate conjugates of steroid hormones and bile salts. Alternative splicing by exon deletion results in several splice variants but maintains the original open reading frame in all forms.

ABCC2 (same as DJS, MRP2, cMRP, ABC30, CMOAT, Canalicular multispecific organic anion transporter) encodes ATP-binding cassette, sub-family C(CFTR/MRP), member 2, and is a useful target locus for the coisogenic cell collections of the present invention. ABCC2 is a member of the MRP subfamily of ATP binding cassette proteins, and is involved in multi-drug resistance. This protein is expressed in the canalicular (apical) part of the hepatocyte and functions in biliary transport. Known substrates include anticancer drugs such as vinblastine.

Another ATP binding cassette protein usefully targeted in the coisogenic cell collections of the present invention is ABCC3 (also known as MLP2, MRP3, ABC31, CMOAT2, MOAT-D, EST90757), the human homologue of which is located at 17q22. The protein may play a role in the transport of biliary and intestinal excretion of organic anions. Alternative splicing of this gene results in three known transcript variants.

Also a useful target for the coisogenic cell collections of the present invention is ATP-binding cassette, sub-family C(CFTR/MRP), member 4, ABCC4, also known as MRP4, MOATB, MOAT-B, EST170205. The protein encoded by this gene is a member of the MRP subfamily of ABC transporters, and is involved in multi-drug resistance. The protein may play a role in cellular detoxification as a pump for its substrate, organic anions.

Other useful ABC transporter proteins that can usefully serve as the target locus for the coisogenic cell collections of the present invention include ABCC4 (MRP4), ABCC5 (MRP5) (provides resistance to thiopurine anticancer drugs, such as 6-mercatopurine and thioguanine, and the anti-HIV drug 9-(2-phosphonylmethoxyethyl)adenine; this protein may be involved in resistance to thiopurines in acute lymphoblastic leukemia and antiretroviral nucleoside analogs in HIV-infected patients); ABCC6 (MRP6), MRP7 (CFTR), ABCC8 (MRP8), ABCC9, ABCC10, ABCC11 (same as HI, SUR, MRP8, PHHI, SUR1, ABC36, HRINS), and ABCC12 (same as MRP9).

Other useful targets include EPHX1 (epoxide hydrolase 1, microsomal xenobiotic), EPHX2 (epoxide hydrolase 2), LTA4H (leukotriene A4 hydrolase), TRAG3 (Taxol® resistance associated gene 3, which is overexpressed in most melanoma cells and confers resistance to paclitaxel, Taxol®), GUSB (beta-glucuronidase), TMPT (thiopurine methyltransferase), BCRP, (breast cancer resistance protein, an ATP transporter), dihydropyrihidine dehydrogenase, HERG (involved in drug transport through potassium ion channels), hKCNE2 (involved in drug transport through potassium ion channels), UDP glucuronosyl transferase (UGT) (a hepatic metabolizing enzyme, a detoxifying enzyme for most carcinogens after different cytochrome P450 (CYP) isoforms), sulfotransferase, sulfatase, and glutathione S-transferase (GST)-alpha, -mu, -pi (which detoxify therapeutic drugs, not least several anti-cancer drugs), ACE (peptidyl-dipeptidase A), and KCHN2 (potassium voltage-gated channel, subfamily H (eag-related), member 2), location 7q35-q36).

Another protein usefully targeted in the coisogenic cell collections of the present invention is the BCR-ABL fusion responsible for chronic myeloid leukemia. The tyrosine kinase domain of the fusion protein is targeted by imatinib (Gleevec); allelic variants have been identified that confer polyclonal resistance to the drug. Shah et al., Cancer Cell 2:117-125 (2002), incorporated herein by reference in its entirety.

Another protein usefully targeted in the coisogenic cell collections of the present invention is beta tubulin. Paclitaxel is a tubulin-disrupting agent that binds preferentially to beta-tubulin. Allelic variants of beta tubulin have been identified that confer resistance to paclitaxel. Giannakakou et al., J. Biol. Chem. 272:17118-17125 (1997), incorporated herein by reference in its entirety.

As noted above, the coisogenic cell collections of the present invention can usefully include cells that have, at the coisogenic target locus, the sequence of a naturally-occurring allele; this permits the phenotype conferred by the allele to be assessed without the confounding presence of other genetic differences at the target locus or elsewhere in the cellular genome. Accordingly, the coisogenic cell collections of the present invention can usefully include cells that have the naturally occurring (allelic) variants set forth in the following tables.

TABLE 1

| Gene | | | mRNA/ | Structural |
|---|---|---|---|---|
| (Synonyms) | Locus | Accession #s | Protein | Information |
| ABCB1 | 7q21.1 | X58723 and | 4643 bp | |
| “ATP-binding | | X59732 | 1279 aa | |
| cassette, sub- | | AC002457, | | |
| family B | | | | |
| (MDR/TAP), | | AC005068 (g) | | |
| member 1” | | AF016535, | | |
| (MDR1, P-GP, | | M14758 (m) | | |
| PGY1, ABC20, | | NP_000918 (p) | | |
| GP170, P- | | | | |
| Glycoprotein) | | | | |

Allelic Variants from Scientific Literature

| | Protein | | |
|---|---|---|---|
| DNA Variant | Variant | Phenotype | References |
| GGA > GTA | Gly185Val | Correlated with | OMIM 171050, Safa et al. |
| | | increased | (1990), Choi et al. (1988) |
| | | colchicine | |
| | | resistance | |
| G2995A | Ala999Thr | Unknown | Mickley et al. (1998) |
| GCT > TCT, | Ala893Ser | “correlations of | Tanabe et al. (2001), Cascorbi et |
| G2677T | | mutations with | al. (2001) |
| | | expression levels” | |
| GCT > ACT, | Ala893Thr | “correlations of | Tanabe et al. (2001), Cascorbi et |
| G2677A | | mutations with | al. (2001) |
| | | expression levels” | |
| AAT > GAT, | Asn21Asp | Unknown | Cascorbi et al. (2001), |
| A61G | | | Hoffmeyer et al. (2000), |
| | | | WO 01/09183 |
| AGT > AAT, | Ser400Asn | “may correlate | Cascorbi et al. (2001), |
| G1199A | | with low | Hoffmeyer et al. (2000), |
| | | expression” WO | WO 01/09183 |
| | | 01/09183 (p40) | |
| CAG > CCG, | Gln1107Pro | Unknown | Cascorbi et al. (2001) |
| A3320C | | | |
| CAG > ???, | Phe103Ser | Unknown | WO 01/09183 (p7) |
| A3320? | | | |
| TTC > CTC, | Phe103Leu | Unknown | Hoffmeyer et al. (2000), |
| T307C | | | WO 01/09183 |
| ATC > ATT, | lle1145lle | Correlated with | OMIM 171050, Hoffmeyer et al. |
| C3435T | (wobble) | (2X) lower p- | (2000) |
| | | glycoprotein | |
| | | expression and | |
| | | activity | |

Allelic Variants from SNP Database

| | | dbSNPrs# | | | | | |
|---|---|---|---|---|---|---|---|
| Contig | Contig | (Cluster | Protein | dbSNP | Protein | Codon | Amino |
| Accession | Position | ID) | Accession | Allele | Residue | Position | Acid |
| NT_017168 | 4730224 | rs2235039 | XP_029059 | g | V | 1 | 801 |
| | | | | a | M | | |
| NT_017168 | 4735268 | rs2032581 | XP_029059 | a | I | 1 | 829 |
| | | | | g | V | | |

TABLE 2

| Gene | | | mRNA/ | Structural |
|---|---|---|---|---|
| (Synonyms) | Locus | Accession #s | Protein | Information |
| ABCB4 | 7q21.1 | NT_017168 | 5764, 5785, l | |
| “ATP-binding | | (working draft | and 5623 bp | |
| cassette, sub- | | chromo7) | 1279, 1286, | |
| family B | | M23234,Z35284 (m) | and 1232 aa | |
| (MDR/TAP), | | | | |
| member 4” | | | | |
| (MDR3, PGY3, | | | | |
| ABC21, | | | | |
| MDR2/3, PFIC- | | | | |
| 3, P- | | | | |
| glycoprotein 3) | | | | |

Allelic Variants from Scientific Literature:

| DNA Variant | Protein Variant | Phenotype | References |
|---|---|---|---|
| CGA > TGA | Arg957Ter | Cholestasis | OMIM 171060 |

Allelic Variants from SNP Database:

| Contig | Contig | dbSNPrs# | Protein | dbSNP | Protein | Codon | Amino |
|---|---|---|---|---|---|---|---|
| Accession | Position | (Cluster ID) | Accession | Allele | Residue | Position | Acid |
| NT_017168 | 4860286 | rs31655 | XP_004599 | g | A | 1 | 1107 |
| | | | | a | T | | |

TABLE 3

| | | | | Struc- |
|---|---|---|---|---|
| | | | | tural |
| | | | | In- |
| Gene | | | mRNA/ | forma- |
| (Synonyms) | Locus | Accession #s | Protein | tion |
| ABCC1 | 16p13.1 | AF022824 (exon2) | 5927, 5749, | |
| “ATP-binding | | AF022825 (exon3) | and 5759 bp | |
| cassette, sub- | | AF022826 (exon4) | 1531, 1472, | |
| family C | | AF022827 (exon5) | and 1475 aa | |
| (CFTR/MRP), | | AF022828 (exon6) | | |
| member 1” | | AF022829 (exon7) | | |
| (MRP, ABCC, | | AF022830 (exon8) | | |
| GS-X, MRP1, | | AF022831 (exon9) | | |
| ABC29) | | AF022832 (exon10) | | |
| | | AF017145 | | |
| | | (5′flanking | | |
| | | sequence) | | |
| | | L05628,U91318 (m) | | |
| | | NP_004987 isoform | | |
| | | 1 (p) | | |
| | | NP_063915 isoform | | |
| | | 2 (p) | | |
| | | NP_063953 isoform | | |
| | | 3 (p) | | |

Allelic Variants from Scientific Literature:

| | Protein | | |
|---|---|---|---|
| DNA Variant | Variant | Phenotype | References |
| G128C | Cys43Ser | Unknown | Ito et al. (2001) |
| C218T | Thr73Ile | Unknown | Ito et al. (2001) |
| G2168A | Arg723Gln | Unknown | Ito et al. (2001) |
| G3173A | Arg1058Gln | Unknown | Ito et al. (2001) |

TABLE 4

| Gene | | | mRNA/ | Structural |
|---|---|---|---|---|
| (Synonyms) | Locus | Accession #s | Protein | Information |
| ABCC2 | 10q24 | NT_029377 | 4868 bp | |
| “ATP-binding | | (working draft | 1545 aa | |
| cassette, sub- | | chromo10) | | |
| family C | | U63970 (m) | | |
| (CFTR/MRP), | | NP_000383 (p) | | |
| member 2” | | | | |
| (DJS, MRP2, | | | | |
| cMRP, ABC30, | | | | |
| CMOAT) | | | | |

Allelic Variants from Scientific Literature:

| | Protein | | |
|---|---|---|---|
| DNA Variant | Variant | Phenotype | References |
| C2302T | Arg768Trp | Dubin-Johnson | OMIM 601107, |
| | | syndrome | Toh et al. (1999), |
| | | | Wada et al. (1998), |
| | | | Ito et al. (2001) |
| A4145G | Gln1382Arg | Dubin-Johnson | OMIM 601107, |
| | | syndrome | Toh et al. (1999). |
| G1249A | Val417Ile | Unknown | Ito et al. (2001) |
| C2366T | Ser789Phe | Unknown | Ito et al. (2001) |
| G4348A | Ala1450Thr | Unknown | Ito et al. (2001) |

TABLE 5

| Gene | | | mRNA/ | Structural |
|---|---|---|---|---|
| (Synonyms) | Locus | Accession #s | Protein | Information |
| ABCC3 | 17q22 | NT_010783 | 5176, 5325, | |
| “ATP-binding | | (working draft | and 5380 bp | |
| cassette, sub- | | chromo17) | 1527, 1238, | |
| family C | | | and 510 aa | |
| (CFTR/MRP), | | AF009670 (m) | | |
| member 3” | | AF085690 (m) | | |
| (MLP2, MRP3, | | AF085691 (m) | | |
| ABC31, | | AF085692 (m) | | |
| CMOAT2, | | NP_003777 (p) | | |
| MOAT-D, | | NP_064421 (p) | | |
| EST90757) | | NP_064422 (p) | | |

Allelic Variants from SNP Database:

| Contig | Contig | dbSNPrs# | Protein | dbSNP | Protein | Codon | Amino |
|---|---|---|---|---|---|---|---|
| Accession | Position | (Cluster ID) | Accession | Allele | Residue | Position | Acid |
| NT_010783 | 1635643 | rs1051625 | XP_008422 | c | L | 1 | 120 |
| | | | | g | V | | |
| NT_010783 | 1619267 | _{—} | XP_037992 | c | T | 2 | 527 |
| | | | | g | R | | |
| NT_010783 | 1619270 | rs1003355 | XP_037992 | c | A | 2 | 528 |
| | | | | g | G | | |
| NT_010783 | 1629592 | rs967935 | XP_037992 | c | S | 2 | 1221 |
| | | | | t | F | | |
| NT_010783 | 1619267 | rs1003354 | XP_037994 | c | T | 2 | 527 |
| | | | | g | R | | |
| NT_010783 | 1619270 | rs1003355 | XP_037994 | c | A | 2 | 528 |
| | | | | g | G | | |
| NT_010783 | 1635643 | rs1051625 | XP_037994 | c | L | 1 | 1362 |
| | | | | g | V | | |
| NT_010783 | 1619267 | rs1003354 | XP_037997 | c | T | 2 | 454 |
| | | | | g | R | | |
| NT_010783 | 1619270 | rs1003355 | XP_037997 | c | A | 2 | 455 |
| | | | | g | G | | |
| NT_010783 | 1635643 | rs1051625 | XP_037997 | c | L | 1 | 1289 |
| | | | | g | V | | |
| NT_010783 | 1619267 | rs1003354 | XP_037999 | c | T | 2 | 527 |
| | | | | g | R | | |
| NT_010783 | 1619270 | rs1003355 | XP_037999 | c | A | 2 | 528 |
| | | | | g | G | | |
| NT_010783 | 1635643 | rs1051625 | XP_037999 | c | L | 1 | 1362 |
| | | | | g | V | | |
| NT_010783 | 1619267 | rs1003354 | XP_038002 | c | T | 2 | 527 |
| | | | | g | R | | |
| NT_010783 | 1619270 | rs1003355 | XP_038002 | c | A | 2 | 528 |
| | | | | g | G | | |
| NT_010783 | 1635643 | rs1051625 | XP_038002 | c | L | 1 | 1362 |
| | | | | g | V | | |

TABLE 6

| Gene | | | mRNA/ | Structural |
|---|---|---|---|---|
| (Synonyms) | Locus | Accession #s | Protein | Information |
| ABCC5 | 3q27 | NT_022676 | 5838 bp | |
| “ATP-binding | | (working draft | 1437 aa | |
| cassette, sub- | | chromo3) | | |
| family C | | NP_005679 (m) | | |
| (CFTR/MRP), | | | | |
| member 5” | | NP_005679 (p) | | |
| (MRP5, SMRP, | | | | |
| ABC33, | | | | |
| MOATC, | | | | |
| MOAT-C, | | | | |
| pABC11, | | | | |
| EST277145) | | | | |

Allelic Variants from SNP Database:

| Contig | Contig | dbSNPrs# | Protein | dbSNP | Protein | Codon | Amino |
|---|---|---|---|---|---|---|---|
| Accession | Position | (Cluster ID) | Accession | Allele | Residue | Position | Acid |
| NT_022676 | 100964 | rs1053351 | XP_002914 | c | Y | 3 | 1202 |
| | | | | a | * | | |
| NT_022676 | 124876 | rs1053387 | XP_002914 | c | T | 2 | 1383 |
| | | | | a | N | | |
| NT_022676 | 100964 | rs1053351 | XP_037577 | c | Y | 3 | 711 |
| | | | | a | * | | |
| NT_022676 | 124876 | rs1053387 | XP_037577 | c | T | 2 | 892 |
| | | | | a | N | | |

TABLE 7

| Gene | | | mRNA/ | Structural |
|---|---|---|---|---|
| (Synonyms) | Locus | Accession #s | Protein | Information |
| ABCC6 | 16p13.1 | U91318 (human | 4535 bp | |
| “ATP-binding | | BAC clone) | 1503 aa | |
| cassette, sub- | | | | |
| family C | | AF076622 (m) | | |
| (CFTR/MRP), | | NP_001162 (p) | | |
| member 6” | | | | |
| (ARA, PXE, | | | | |
| MLP1, MRP6, | | | | |
| ABC34, | | | | |
| MOATE, | | | | |
| EST349056) | | | | |

Allelic Variants from Scientific Literature:

| | Protein | | |
|---|---|---|---|
| DNA Variant | Variant | Phenotype | References |
| C3421T | Arg1141Ter | Pseudoxanthoma | OMIM 603234 |
| | | Elasticum | |
| G3413A | Arg1138Gln | Pseudoxanthoma | OMIM 603234 |
| | | Elasticum | |
| G3341C | Arg1114Pro | Pseudoxanthoma | OMIM 603234 |
| | | Elasticum | |
| C3940T | Arg1314Trp | Pseudoxanthoma | OMIM 603234 |
| | | Elasticum | |
| — | Arg1268Gln | Pseudoxanthoma | OMIM 603234 |
| | | Elasticum | |
| C3412T | Arg1138Trp | Pseudoxanthoma | OMIM 603234 |
| | | Elasticum | |
| C3490T | Arg1164Ter | Pseudoxanthoma | OMIM 603234 |
| | | Elasticum | |

Allelic Variants from SNP Database:

| Contig | Contig | dbSNPrs# | Protein | dbSNP | Protein | Codon | Amino |
|---|---|---|---|---|---|---|---|
| Accession | Position | (Cluster ID) | Accession | Allele | Residue | Position | Acid |
| NT_010393 | 2241302 | rs2238472 | XP_007798 | g | R | 2 | 1268 |
| | | | | a | Q | | |
| NT_010393 | 2241302 | rs2238472 | XP_027249 | g | R | 2 | 33 |
| | | | | a | Q | | |

TABLE 8

| Gene | | | mRNA/ | Structural |
|---|---|---|---|---|
| (Synonyms) | Locus | Accession #s | Protein | Information |
| ABCC8 | 11p15.1 | L78243 (exon39) | 4977 bp | |
| “ATP-binding | | U63455 (exon39 | 1581 aa | |
| cassette, sub- | | and complete cds.) | | |
| family C | | NT_009307 | | |
| (CFTR/MRP), | | (working draft | | |
| member 8” | | chromo11) | | |
| (HI, SUR, | | AH004854 (m) | | |
| MRP8, PHHI, | | NP_000343 (p) | | |
| SUR1, ABC36, | | | | |
| HRINS) | | | | |

Allelic Variants from Scientific Literature:

| | Protein | | |
|---|---|---|---|
| DNA Variant | Variant | Phenotype | References |
| G > T | Gly716Val | Persistent | OMIM 600509 |
| | | Hyperinsulinemic | |
| | | Hypoglycemia of | |
| | | Infancy | |
| G4058C | Arg1353Pro | Persistent | OMIM 600509 |
| | | Hyperinsulinemic | |
| | | Hypoglycemia of | |
| | | Infancy | |
| C4261T | Arg1421Cys | Persistent | OMIM 600509 |
| | | Hyperinsulinemic | |
| | | Hypoglycemia of | |
| | | Infancy | |
| C4480T | Arg1494Trp | Persistent | OMIM 600509 |
| | | Hyperinsulinemic | |
| | | Hypoglycemia of | |
| | | Infancy | |

Allelic Variants from SNP Database:

| Contig | Contig | dbSNPrs# | Protein | dbSNP | Protein | Codon | Amino |
|---|---|---|---|---|---|---|---|
| Accession | Position | (Cluster ID) | Accession | Allele | Residue | Position | Acid |
| NT_009307 | 1343092 | rs1048098 | XP_036346 | t | F | 1 | 157 |
| | | | | c | L | | |
| NT_009307 | 1343122 | rs1048096 | XP_036346 | c | L | 1 | 167 |
| | | | | g | V | | |
| NT_009307 | 1344909 | rs1048095 | XP_036346 | t | L | 2 | 225 |
| | | | | c | P | | |
| NT_009307 | 1345002 | rs1048094 | XP_036346 | c | A | 2 | 256 |
| | | | | t | V | | |
| NT_009307 | 1409710 | rs757110 | XP_036346 | g | A | 1 | 1369 |
| | | | | t | S | | |

TABLE 9

| Gene | | | mRNA/ | Structural |
|---|---|---|---|---|
| (Synonyms) | Locus | Accession #s | Protein | Information |
| ACE | 17q23 | NT_010698 | 4020 bp | |
| “angiotensin I | | (working draft | 1306 aa | |
| converting | | chromo17) | | |
| enzyme | | J04144 (m) | | |
| (peptidyl- | | NP_000780 (p) | | |
| dipeptidase A)1” | | | | |
| (ACE1, DCP1, | | | | |
| CD143) | | | | |

Allelic Variants from Scientific Literature:

| | Protein | | |
|---|---|---|---|
| DNA Variant | Variant | Phenotype | References |
| A2350G | ? | “significantly | OMIM 106180 |
| | | associated with | |
| | | blood pressure” | |

Allelic Variants from SNP Database:

| Contig | Contig | dbSNPrs# | Protein | dbSNP | Protein | Codon | Amino |
|---|---|---|---|---|---|---|---|
| Accession | Position | (Cluster ID) | Accession | Allele | Residue | Position | Acid |
| NT_010698 | 1458291 | rs4348 | XP_008260 | c | P | 2 | 5 |
| | | | | t | L | | |
| NT_010698 | 1460620 | rs4976 | XP_008260 | t | I | 2 | 94 |
| | | | | c | T | | |

TABLE 10

| Gene | | | mRNA/ | Structural |
|---|---|---|---|---|
| (Synonyms) | Locus | Accession #s | Protein | Information |
| CYP1A1 | 15q22-24 | X02612 | 2602 bp | |
| “cytochrome | | X04300 | 512 aa | |
| P450, | | X02612 (m) | | |
| subfamily I | | X04300 (m) | | |
| (aromatic | | NP_000490 (p) | | |
| compound- | | | | |
| inducible), | | | | |
| polypeptide1” | | | | |
| (AHH, AHRR, | | | | |
| CP11, CYP1, | | | | |
| P1-450, P450- | | | | |
| C, P450DX) | | | | |

Allelic Variants from Scientific Literature:

| | Protein | | |
|---|---|---|---|
| DNA Variant | Variant | Phenotype | References |
| ? | Ala462Val | Correlated with | OMIM 108330 |
| | | increased risk of | |
| | | lung cancer, but may | |
| | | be just marker | |

Allelic Variants from SNP Database:

| Contig | Contig | dbSNPrs# | Protein | dbSNP | Protein | Codon | Amino |
|---|---|---|---|---|---|---|---|
| Accession | Position | (Cluster ID) | Accession | Allele | Residue | Position | Acid |
| NT_010374 | 225016 | rs1048943 | XP_007727 | a | I | 1 | 462 |
| | | | | g | V | | |
| NT_010374 | 225018 | rs1799814 | XP_007727 | c | T | 2 | 461 |
| | | | | a | N | | |
| NT_010374 | 227193 | rs2229150 | XP_007727 | c | R | 1 | 93 |
| | | | | t | W | | |

TABLE 11

| Gene | | | mRNA/ | Structural |
|---|---|---|---|---|
| (Synonyms) | Locus | Accession #s | Protein | Information |
| CYP1B1 | 2p21 | U56438 | 5128 bp | |
| “cytochrome | | | 543 aa | |
| P450, | | X04300 (g) | | |
| subfamily I | | X02612 (g) | | |
| (dioxin- | | U56438 (g) | | |
| inducible), | | U03688 (m) | | |
| polypeptide 1 | | NP_000095 (p) | | |
| (glaucoma 3, | | | | |
| primary | | | | |
| infantile)” | | | | |
| (CP1B, | | | | |
| GLC3A) | | | | |

Allelic Variants from Scientific Literature:

| | Protein | | |
|---|---|---|---|
| DNA Variant | Variant | Phenotype | References |
| G3976A | Trp57Ter | Peters Anomaly | OMIM 601771 |
| T3807C | Met1Thr | Peters Anomaly | OMIM 601771 |
| G1505A | Lys387Gln | Glaucoma | OMIM 601771 |
| G7957A | Asp374Asn | Glaucoma | OMIM 601771 |
| C8242T | Arg469Trp | Glaucoma | OMIM 601771 |
| G3987A | Gly61Glu | Glaucoma | OMIM 601771 |
| ? | Gly365Trp | Glaucoma | OMIM 601771 |

Allelic Variants from SNP Database:

| Contig | Contig | dbSNPrs# | Protein | dbSNP | Protein | Codon | Amino |
|---|---|---|---|---|---|---|---|
| Accession | Position | (Cluster ID) | Accession | Allele | Residue | Position | Acid |
| NT_005274 | 679631 | rs10012 | XP_002576 | c | R | 1 | 48 |
| | | | | g | G | | |
| NT_005274 | 679844 | rs1056827 | XP_002576 | g | A | 1 | 119 |
| | | | | t | S | | |
| NT_005274 | 683818 | rs1056836 | XP_002576 | g | V | 1 | 432 |
| | | | | c | L | | |
| NT_005274 | 683871 | rs1056837 | XP_002576 | t | D | 3 | 449 |
| | | | | a | E | | |
| NT_005274 | 683882 | rs1800440 | XP_002576 | a | N | 2 | 453 |
| | | | | g | S | | |

TABLE 12

| Gene | | | mRNA/ | Structural |
|---|---|---|---|---|
| (Synonyms) | Locus | Accession #s | Protein | Information |
| CYP2A6 | 19q13.2 | U22027 | 1751 bp | |
| “cytochrome | | | 494 aa | |
| P450, | | NM_000762 (m) | | |
| subfamily IIA | | NP_000753 (p) | | |
| (phenobarbital- | | NG_000008 (g) | | |
| inducible), | | | | |
| polypeptide 6” | | | | |
| (CPA6, | | | | |
| CYP2A3) | | | | |

Allelic Variants from Scientific Literature:

| | Protein | | |
|---|---|---|---|
| DNA Variant | Variant | Phenotype | References |
| ? | Leu160His | Protein becomes | OMIM 601771 |
| | | “catalytically | |
| | | inactive” | |

TABLE 13

| Gene | | | mRNA/ | Structural |
|---|---|---|---|---|
| (Synonyms) | Locus | Accession #s | Protein | Information |
| CYP2A7 | 19q13.2 | NT_029481 | 2281 bp and | |
| “cytochrome | | (working draft | 2128 bp | |
| P450, | | chromo19) | 494 and 443 | |
| | | | aa | |
| subfamily IIA | | | | |
| (phenobarbital- | | NG_000008 (g) | | |
| inducible), | | NM_000764 (m) | | |
| polypeptide 7” | | NP_000755 (p) | | |
| (CPA7, CPAD, | | NP085079 (p) | | |
| CYPIIA7, | | | | |
| P450-IIA4) | | | | |

Allelic Variants from Scientific Literature:

| | Protein | | |
|---|---|---|---|
| DNA Variant | Variant | Phenotype | References |
| T > A | Leu160His | Uknown | OMIM 122720 |

TABLE 14

| Gene | | | mRNA/ | Structural |
|---|---|---|---|---|
| (Synonyms) | Locus | Accession #s | Protein | Information |
| CYP2C8 | 10cen- | L16876 (exon 9) | 1851 and 1890 bp | |
| “cytochrome | q26.11 | NT_008769 | | |
| P450, | | (working draft 10) | 490 and 393 aa | |
| subfamily IIC | | | | |
| (mephenytoin | | NM_000770 (m) | | |
| 4-hydroxylase), | | NM_030878 (m) | | |
| polypeptide 8” | | NP_000761 (p) | | |
| (CPC8, | | NP_110518 (p) | | |
| P450 MP- | | | | |
| 12/MP-20) | | | | |

Allelic Variants from SNP Database:

| Contig | Contig | dbSNPrs# | Protein | dbSNP | Protein | Codon | Amino |
|---|---|---|---|---|---|---|---|
| Accession | Position | (Cluster ID) | Accession | Allele | Residue | Position | Acid |
| NT_008769 | 823719 | rs1058930 | XP_011938 | c | I | 3 | 264 |
| | | | | g | M | | |
| NT_008769 | 823719 | rs1058930 | XP_050924 | c | I | 3 | 67 |
| | | | | g | M | | |
| NT_008769 | 823719 | rs1058930 | XP_050926 | c | I | 3 | 251 |
| | | | | g | M | | |
| NT_008769 | 823719 | rs1058930 | XP_050929 | c | I | 3 | 264 |
| | | | | g | M | | |

TABLE 15

| Gene | | | mRNA/ | Structural |
|---|---|---|---|---|
| (Synonyms) | Locus | Accession #s | Protein | Information |
| CYP2C9 | 10q24 | NT_008769 | 1835 bp | |
| “cytochrome | | (working draft | 490 aa | |
| P450, | | chromo10) | | |
| subfamily IIC | | | | |
| (mephenytoin | | NM_000771 (m) | | |
| 4-hydroxylase), | | NP_000762 (p) | | |
| polypeptide 9” | | | | |
| (CPC9, | | | | |
| CYP2C10, | | | | |
| P450IIC9, | | | | |
| P450 MP-4, | | | | |
| P450 PB-1) | | | | |

Allelic Variants from Scientific Literature:

| | Protein | | |
|---|---|---|---|
| DNA Variant | Variant | Phenotype | References |
| ? | Arg144Cys | Warfarin Sensitivity | OMIM 601129 |
| ? | Ile359Leu | Poor tolbutamide | OMIM 601129 |
| | | metabolism | |
| | | (diabetes mellitus) | |

Allelic Variants from SNP Database:

| Contig | Contig | dbSNPrs# | Protein | dbSNP | Protein | Codon | Amino |
|---|---|---|---|---|---|---|---|
| Accession | Position | (Cluster ID) | Accession | Allele | Residue | Position | Acid |
| NT_008769 | 43400 | rs1057910 | XP_050915 | a | I | 1 | 21 |
| | | | | c | L | | |
| NT_008769 | 43402 | rs1057909 | XP_050915 | a | Y | 2 | 20 |
| | | | | g | C | | |

TABLE 16

| Gene | | | mRNA/ | Structural |
|---|---|---|---|---|
| (Synonyms) | Locus | Accession #s | Protein | Information |
| CYP2C19 | 10q24.1- | NT_008769 | 1473 bp | arg433-to-trp |
| “cytochrome | q24.3 | (working draft | 490 aa | mutation in the |
| P450, | | chromo10) | | heme-binding |
| subfamily IIC | | M61854 (m) | | region |
| (mephenytoin | | NM_000769 (m) | | |
| 4-hydroxylase), | | NP_000760 (p) | | Ibeanu et al. |
| polypeptide | | | | (1998) |
| 19” | | | | |
| (CPCJ, | | | | |
| CYP2C, | | | | |
| P450C2C, | | | | |
| P450IIC19) | | | | |

Allelic Variants from Scientific Literature:

| | Protein | | |
|---|---|---|---|
| DNA Variant | Variant | Phenotype | References |
| ? | Arg433Trp | Mephenytoin 4- | OMIM 124020 |
| | | Hydroxylase defect, | |
| | | poor metabolizer | |

TABLE 17

| Gene | | | mRNA/ | Structural |
|---|---|---|---|---|
| (Synonyms) | Locus | Accession #s | Protein | Information |
| CYP2D6 | 22q13.1 | M33388 | 1655 bp | |
| “cytochrome | | NM_000106 (m) | 497 aa | |
| P450, | | NP_000097 (p) | | |
| subfamily IID | | | | |
| (debrisoquine, | | | | |
| sparteine, etc.,- | | | | |
| metabolizing), | | | | |
| polypeptide 6” | | | | |
| (CPD6, | | | | |
| CYP2D, | | | | |
| CYP2D@, | | | | |
| P450C2D, | | | | |
| P450-DB1) | | | | |

Allelic Variants from Scientific Literature:

| | Protein | | |
|---|---|---|---|
| DNA Variant | Variant | Phenotype | References |
| ? | Gly169Ter | Debrisoquine, poor | OMIM 124030 |
| | | drug metabolizer | |

Allelic Variants from SNP Database:

| Contig | Contig | dbSNPrs# | Protein | dbSNP | Protein | Codon | Amino |
|---|---|---|---|---|---|---|---|
| Accession | Position | (Cluster ID) | Accession | Allele | Residue | Position | Acid |
| NT_011520 | 21651359 | rs2103556 | XP_013013 | c | T | 2 | 396 |
| | | | | g | S | | |
| NT_011520 | 21651463 | rs2070905 | XP_013013 | g | M | 3 | 361 |
| | | | | a | I | | |
| NT_011520 | 21651686 | rs2070907 | XP_013013 | a | K | 1 | 320 |
| | | | | g | E | | |
| NT_011520 | 21652249 | rs1065569 | XP_013013 | g | V | 1 | 284 |
| | | | | a | M | | |
| NT_011520 | 21652275 | rs1974456 | XP_013013 | g | R | 2 | 275 |
| | | | | a | H | | |
| NT_011520 | 21652631 | rs1800754 | XP_013013 | c | S | 2 | 221 |
| | | | | t | L | | |
| NT_011520 | 21652662 | rs1058171 | XP_013013 | a | N | 1 | 211 |
| | | | | g | D | | |
| NT_011520 | 21652664 | rs1058170 | XP_013013 | g | G | 2 | 210 |
| | | | | c | A | | |
| NT_011520 | 21653063 | rs1058167 | XP_013013 | c | P | 2 | 141 |
| | | | | t | L | | |
| NT_011520 | 21651359 | rs2103556 | XP_040060 | c | T | 2 | 140 |
| | | | | g | S | | |
| NT_011520 | 21651463 | rs2070905 | XP_040060 | g | M | 3 | 105 |
| | | | | a | I | | |
| NT_011520 | 21651686 | rs2070907 | XP_040060 | a | K | 1 | 64 |
| | | | | g | E | | |
| NT_011520 | 21652249 | rs1065569 | XP_040060 | g | V | 1 | 28 |
| | | | | a | M | | |
| NT_011520 | 21652275 | rs1974456 | XP_040060 | g | R | 2 | 19 |
| | | | | a | H | | |
| NT_011520 | 21651359 | rs2103556 | XP_040062 | c | T | 2 | 140 |
| | | | | g | S | | |
| NT_011520 | 21651463 | rs2070905 | XP_040062 | g | M | 3 | 105 |
| | | | | a | I | | |
| NT_011520 | 21651686 | rs2070907 | XP_040062 | a | K | 1 | 64 |
| | | | | g | E | | |
| NT_011520 | 21652249 | rs1065569 | XP_040062 | g | V | 1 | 28 |
| | | | | a | M | | |
| NT_011520 | 21652275 | rs1974456 | XP_040062 | g | R | 2 | 19 |
| | | | | a | H | | |
| NT_011520 | 21651359 | rs2103556 | XP_040064 | c | T | 2 | 180 |
| | | | | g | S | | |
| NT_011520 | 21651463 | rs2070905 | XP_040064 | g | M | 3 | 145 |
| | | | | a | I | | |
| NT_011520 | 21651686 | rs2070907 | XP_040064 | a | K | 1 | 104 |
| | | | | g | E | | |
| NT_011520 | 21652249 | rs1065569 | XP_040064 | g | V | 1 | 68 |
| | | | | a | M | | |
| NT_011520 | 21652275 | rs1974456 | XP_040064 | g | R | 2 | 59 |
| | | | | a | H | | |
| NT_011520 | 21652631 | rs1800754 | XP_040064 | c | S | 2 | 5 |
| | | | | t | L | | |
| NT_011520 | 21651359 | rs2103556 | XP_040065 | c | T | 2 | 227 |
| | | | | g | S | | |
| NT_011520 | 21651463 | rs2070905 | XP_040065 | g | M | 3 | 192 |
| | | | | a | I | | |
| NT_011520 | 21651686 | rs2070907 | XP_040065 | a | K | 1 | 151 |
| | | | | g | E | | |
| NT_011520 | 21652249 | rs1065569 | XP_040065 | g | V | 1 | 115 |
| | | | | a | M | | |
| NT_011520 | 21652275 | rs1974456 | XP_040065 | g | R | 2 | 106 |
| | | | | a | H | | |
| NT_011520 | 21652631 | rs1800754 | XP_040065 | c | S | 2 | 52 |
| | | | | t | L | | |
| NT_011520 | 21652662 | rs1058171 | XP_040065 | a | N | 1 | 42 |
| | | | | g | D | | |
| NT_011520 | 21652664 | rs1058170 | XP_040065 | g | G | 2 | 41 |
| | | | | c | A | | |
| NT_011520 | 21651359 | rs2103556 | XP_040066 | c | T | 2 | 396 |
| | | | | g | S | | |
| NT_011520 | 21651463 | rs2070905 | XP_040066 | g | M | 3 | 361 |
| | | | | a | I | | |
| NT_011520 | 21651686 | rs2070907 | XP_040066 | a | K | 1 | 320 |
| | | | | g | E | | |
| NT_011520 | 21652249 | rs1065569 | XP_040066 | g | V | 1 | 284 |
| | | | | a | M | | |
| NT_011520 | 21652275 | rs1974456 | XP_040066 | g | R | 2 | 275 |
| | | | | a | H | | |
| NT_011520 | 21652631 | rs1800754 | XP_040066 | c | S | 2 | 221 |
| | | | | t | L | | |
| NT_011520 | 21652662 | rs1058171 | XP_040066 | a | N | 1 | 211 |
| | | | | g | D | | |
| NT_011520 | 21652664 | rs1058170 | XP_040066 | g | G | 2 | 210 |
| | | | | c | A | | |
| NT_011520 | 21653059 | rs1058169 | XP_040066 | c | H | 3 | 142 |
| | | | | t | H | | |
| NT_011520 | 21653063 | rs1058167 | XP_040066 | c | P | 2 | 141 |
| | | | | t | L | | |

Allelic variants from Karolinska Institute:

| | | Nucleotide_{—} | Trivial | | Enzyme activity | | |
|---|---|---|---|---|---|---|---|
| Allele | Protein | changes | name | Effect | In vivo | In vitro | References |
| CYP2D6*1A | CYP2D6.1 | None | Wild-type | . | Normal | Normal | Kimura et |
| | | | | | | | al, 1989 |
| CYP2D6*2A | CYP2D6.2 | −1584CG; | CYP2D6L | R296C; | Normal | | Johansson |
| | | −1235AG; | | S486T | (dx, d, s) | | et al, 1993 |
| | | −740CT; | | | | | Panserat |
| | | −678GA; | | | | | et al, 1994 |
| | | 1661GC; | | | | | Raimundo |
| | | 2850CT; | | | | | et al, 2000 |
| | | 4180GC | | | | | See also |
| | | | | | | | comment |
| | | | | | | | below the |
| | | | | | | | table. |
| CYP2D6*2B | CYP2D6.2 | 1039CT; | . | R296C; | . | . | Marez et |
| | | 1661GC; | | S486T | | | al, 1997 |
| | | 2850CT; | | | | | |
| | | 4180GC | | | | | |
| CYP2D6*2C | CYP2D6.2 | 1661GC; | . | R296C; | . | . | Marez et |
| | | 2470TC; | | S486T | | | al, 1997 |
| | | 2850CT; | | | | | Sachse et |
| | | 4180GC | | | | | al, 1997 |
| CYP2D6*2D | CYP2D6.2 | 2850CT; | M10 | R296C; | . | . | Marez et |
| | | 4180GC | | S486T | | | al, 1997 |
| CYP2D6*2E | CYP2D6.2 | 997CG; | M12 | R296C; | . | . | Marez et |
| | | 1661GC; | | S486T | | | al, 1997 |
| | | 2850CT; | | | | | |
| | | 4180GC | | | | | |
| CYP2D6*2F | CYP2D6.2 | 1661GC; | M14 | R296C; | . | . | Marez et |
| | | 1724CT; | | S486T | | | al, 1997 |
| | | 2850CT; | | | | | |
| | | 4180GC | | | | | |
| CYP2D6*2G | CYP2D6.2 | 1661GC; | M16 | R296C; | . | . | Marez et |
| | | 2470TC; | | S486T | | | al, 1997 |
| | | 2575CA; | | | | | |
| | | 2850CT; | | | | | |
| | | 4180GC | | | | | |
| CYP2D6*2H | CYP2D6.2 | 1661GC; | M17 | R296C; | . | . | Marez et |
| | | 2480CT; | | S486T | | | al, 1997 |
| | | 2850CT; | | | | | |
| | | 4180GC | | | | | |
| CYP2D6*2J | CYP2D6.2 | 1661GC; | M18 | R296C; | . | . | Marez et |
| | | 2850CT; | | S486T | | | al, 1997 |
| | | 2939GA; | | | | | |
| | | 4180GC | | | | | |
| CYP2D6*2K | CYP2D6.2 | 1661GC; | M21 | R296C; | . | . | Marez et |
| | | 2850CT; | | S486T | | | al, 1997 |
| | | 4115CT; | | | | | |
| | | 4180GC | | | | | |
| CYP2D6*2XN | CYP2D6.2 | 1661GC; | . | R296C; | Incr | . | Johansson |
| | | 2850CT; | | S486T | (d) | | et al, 1993 |
| (N = 2, 3, 4, | | 4180GC | | N active | | | Dahl et al, |
| 5 or 13) | | | | genes | | | 1995 |
| | | | | | | | Aklillu et al, |
| | | | | | | | 1996 |
| CYP2D6*3A | . | 2549Adel | CYP2D6A | Frameshift | None | None | Kagimoto |
| | | | | | (d, s) | (b) | et al, 1990 |
| CYP2D6*3B | . | 1749AG; | . | N166D; | . | . | Marez et |
| | | 2549Adel | | frameshift | | | al, 1997 |
| CYP2D6*4A | . | 100CT; | CYP2D6B | P34S; | None | None | Kagimoto |
| | | 974CA; | | L91M; | (d, s) | (b) | et al, 1990 |
| | | 984AG;_9 | | H94R; | | | Gough et |
| | | 97CG; | | Splicing | | | al, 1990 |
| | | 1661GC; | | defect; | | | Hanioka et |
| | | 1846GA; | | S486T | | | al, 1990 |
| | | 4180GC | | | | | |
| CYP2D6*4B | . | 100CT; | CYP2D6B | P34S; | None | None | Kagimoto |
| | | 974CA; | | L91M; | (d, s) | (b) | et al, 1990 |
| | | 984AG; | | H94R; | | | |
| | | 997CG; | | Splicing | | | |
| | | 1846GA; | | defect; | | | |
| | | 4180GC | | S486T | | | |
| CYP2D6*4C | . | 100CT; | K29-1 | P34S; | None | . | Yokota et |
| | | 1661GC; | | Splicing | | | al, 1993 |
| | | 1846GA; | | defect; | | | |
| | | 3887TC; | | L421P; | | | |
| | | 4180GC | | S486T | | | |
| CYP2D6*4D | . | 100CT; | . | P34S; | None (dx) | . | Marez et |
| | | 1039CT; | | Splicing | | | al, 1997 |
| | | 1661GC; | | defect; | | | |
| | | 1846GA; | | S486T | | | |
| | | 4180GC | | | | | |
| CYP2D6*4E | . | 100CT; | . | P34S; | . | . | Marez et |
| | | 1661GC; | | Splicing | | | al, 1997 |
| | | 1846GA; | | defect; | | | |
| | | 4180GC | | S486T | | | |
| CYP2D6*4F | . | 100CT; | . | P34S; | . | . | Marez et |
| | | 974CA; | | L91M; | | | al, 1997 |
| | | 984AG; | | H94R; | | | |
| | | 997CG; | | Splicing | | | |
| | | 1661GC; | | defect; | | | |
| | | 1846GA; | | R173C; | | | |
| | | 1858CT; | | S486T | | | |
| | | 4180GC | | | | | |
| CYP2D6*4G | . | 100CT; | . | P34S; | . | . | Marez et |
| | | 974CA; | | L91M; | | | al, 1997 |
| | | 984AG; | | H94R; | | | |
| | | 997CG; | | Splicing | | | |
| | | 1661GC; | | defect; | | | |
| | | 1846GA; | | P325L; | | | |
| | | 2938CT; | | S486T | | | |
| | | 4180GC | | | | | |
| CYP2D6*4H | . | 100CT; | . | P34S; | . | . | Marez et |
| | | 974CA; | | L91M; | | | al, 1997 |
| | | 984AG; | | H94R; | | | |
| | | 997CG; | | Splicing | | | |
| | | 1661GC; | | defect; | | | |
| | | 1846GA; | | E418Q; | | | |
| | | 3877GC; | | S486T | | | |
| | | 4180GC | | | | | |
| CYP2D6*4J | . | 100CT; | . | P34S; | . | . | Marez et |
| | | 974CA; | | L91M; | | | al, 1997 |
| | | 984AG; | | H94R; | | | |
| | | 997CG; | | Splicing | | | |
| | | 1661GC; | | defect | | | |
| | | 1846GA | | | | | |
| CYP2D6*4K | . | 100CT; | . | P34S; | None | . | Sachse et |
| | | 1661GC; | | Splicing | | | al, 1997 |
| | | 1846GA; | | defect; | | | |
| | | 2850CT; | | R296C; | | | |
| | | 4180GC | | S486T | | | |
| CYP2D6*4L | | 100CT; | | P34S; | | | Submitted |
| | | 997CG; | | Splicing | | | 17-Aug-00 |
| | | 1661GC; | | defect; | | | by Dr. T. |
| | | 1846GA; | | S486T | | | Shimada |
| | | 4180GC | | | | | |
| CYP2D6*4X2 | . | . | . | . | None | . | Løvlie et |
| | | | | | | | al, 1997 |
| | | | | | | | Sachse et |
| | | | | | | | al, 1998 |
| CYP2D6*5 | . | CYP2D6 | CYP2D6D | CYP2D6 | None | . | Gaedigk et |
| | | deleted | | deleted | (d, s) | | al, 1991 |
| | | | | | | | Steen et al, |
| | | | | | | | 1995 |
| CYP2D6*6A | . | 1707Tdel | CYP2D6T | Frameshift | None | . | Saxena et |
| | | | | | (d, dx) | | al, 1994 |
| CYP2D6*6B | . | 1707Tdel; | . | Frameshift; | None | . | Evert et al, |
| | | 1976GA | | G212E | (s, d) | | 1994 |
| | | | | | | | Daly et al, |
| | | | | | | | 1995 |
| CYP2D6*6C | . | 1707Tdel; | . | Frameshift; | None (s) | . | Marez et |
| | | 1976GA; | | G212E; | | | al, 1997 |
| | | 4180GC | | S486T | | | |
| CYP2D6*6D | . | 1707Tdel; | . | Frameshift; | . | . | Marez et |
| | | 3288GA | | G373S | | | al, 1997 |
| CYP2D6*7 | CYP2D6.7 | 2935AC | CYP2D6E | H324P | None | . | Evert et al, |
| | | | | | (s) | | 1994 |
| CYP2D6*8 | . | 1661GC; | CYP2D6G | Stop | None | . | Broly et al, |
| | | 1758GT; | | codon; | (d, s) | | 1995 |
| | | 2850CT; | | R296C; | | | |
| | | 4180GC | | S486T | | | |
| CYP2D6*9 | CYP2D6.9 | 2613-2615 | CYP2D6C | K281del | Decr | Decr | Tyndale et |
| | | delAGA | . | | (b, s, d) | (b, s, d) | al, 1991 |
| | | | | | | | Broly & |
| | | | | | | | Meyer, |
| | | | | | | | 1993 |
| CYP2D6*10A | CYP2D6.10 | 100CT; | CYP2D6J | P34S; | Decr | . | Yokota et |
| | | 1661GC; | | S486T | (s) | | al, 1993 |
| | | 4180GC | | | | | |
| CYP2D6*10B | CYP2D6.10 | 100CT; | CYP2D6C | P34S; | Decr | Decr | Johansson |
| | | 1039CT; | h1 | S486T | (d) | (b) | et al, 1994 |
| | | 1661GC; | | | | | |
| | | 4180GC | | | | | |
| CYP2D6*10C | | | see CYP2D6*36 | | | | |
| CYP2D6*11 | . | 883GC; | CYP2D6F | Splicing | None | . | Marez et |
| | | 1661GC; | | defect; | (s) | | al, 1995 |
| | | 2850CT; | | R296C; | | | |
| | | 4180GC | | S486T | | | |
| CYP2D6*12 | CYP2D6.12 | 124GA; | . | G42R;; | None | . | Marez et |
| | | 1661GC; | | R296C; | (s) | | al, 1996 |
| | | 2850CT; | | S486T | | | |
| | | 4180GC | | | | | |
| CYP2D6*13 | . | CYP2D7P/ | . | Frameshift | None | . | Panserat |
| | | CYP2D6 | | | (dx) | | et al, 1995 |
| | | hybrid. | | | | | |
| | | Exon 1 | | | | | |
| | | CYP2D7, | | | | | |
| | | exons 2-9 | | | | | |
| | | CYP2D6. | | | | | |
| CYP2D6*14 | CYP2D6.14 | 100CT; | . | P34S; | None | . | Wang, |
| | | 1758GA; | | G169R; | (d) | | 1992 |
| | | 2850CT; | | R296C; | | | Wang et al, |
| | | 4180GC | | S486T | | | 1999 |
| CYP2D6*15 | . | 138insT | . | Frameshift | None | . | Sachse et |
| | | | | | (d, dx) | | al, 1996 |
| CYP2D6*16 | . | CYP2D7P/ | CYP2D6D2 | Frameshift | None | . | Daly et al, |
| | | CYP2D6 | | | (d) | | 1996 |
| | | hybrid. | | | | | |
| | | Exons1-7 | | | | | |
| | | CYP2D7P- | | | | | |
| | | related, | | | | | |
| | | exons 8-9 | | | | | |
| | | CYP2D6. | | | | | |
| CYP2D6*17 | CYP2D6.17 | 1023CT; | CYP2D6Z | T107I; | Decr | Decr | Masimirem |
| | | 1638GC: | | R296C; | (d) | (b) | bwa et al, |
| | | 2850CT; | | S486T | | | 1996 |
| | | 4180GC | | | | | Oscarson |
| | | | | | | | et al, 1997 |
| CYP2D6*18 | CYP2D6.18 | 4125-4133 | CYP2D6(J | 468-470 | None (s) | Decr (b) | Yokoi et al, |
| | | insGT | 9) | VPT | | | 1996 |
| | | GCCCACT | | ins | | | |
| CYP2D6*19 | . | 1661GC; | . | Frameshift; | None | . | Marez et |
| | | 2539-2542 | | R296C; | | | al, 1997 |
| | | delAA | | S486T | | | |
| | | CT; | | | | | |
| | | 2850CT; | | | | | |
| | | 4180GC | | | | | |
| CYP2D6*20 | . | 1661GC; | . | Frameshift; | None (m) | . | Marez- |
| | | 1973insG; | | L213S; | | | Allorge et |
| | | 1978CT; | | R296C; | | | al, 1999 |
| | | 1979TC; | | S486T | | | |
| | | 2850CT; | | | | | |
| | | 4180GC | | | | | |
| CYP2D6*21 | CYP2D6.21 | 77GA | M1 | R26H | . | . | Marez et |
| | | | | | | | al, 1997 |
| CYP2D6*22 | CYP2D6.22 | 82CT | M2 | R28C | . | . | Marez et |
| | | | | | | | al, 1997 |
| CYP2D6*23 | CYP2D6.23 | 957CT | M3 | A85V | . | . | Marez et |
| | | | | | | | al, 1997 |
| CYP2D6*24 | CYP2D6.24 | 2853AC | M6 | I297L | . | . | Marez et |
| | | | | | | | al, 1997 |
| CYP2D6*25 | CYP2D6.25 | 3198CG | M7 | R343G | . | . | Marez et |
| | | | | | | | al, 1997 |
| CYP2D6*26 | CYP2D6.26 | 3277TC | M8 | I369T | . | . | Marez et |
| | | | | | | | al, 1997 |
| CYP2D6*27 | CYP2D6.27 | 3853GA | M9 | E410K | . | . | Marez et |
| | | | | | | | al, 1997 |
| CYP2D6*28 | CYP2D6.28 | 19GA; | M11 | V7M; | . | . | Marez et |
| | | 1661GC; | | Q151E; | | | al, 1997 |
| | | 1704CG; | | R296C; | | | |
| | | 2850CT; | | S486T | | | |
| | | 4180GC | | | | | |
| CYP2D6*29 | CYP2D6.29 | 1659GA; | M13 | V136M; | . | . | Marez et |
| | | 1661GC; | | R296C; | | | al, 1997 |
| | | 2850CT; | | V338M; | | | |
| | | 3183GA; | | S486T | | | |
| | | 4180GC | | | | | |
| CYP2D6*30 | CYP2D6.30 | 1661GC; | M15 | 172-174 | . | . | Marez et |
| | | 1863 ins | | FRP | | | al, 1997 |
| | | 9bp rep; | | rep; | | | |
| | | 2850CT; | | R296C; | | | |
| | | 4180GC | | S486T | | | |
| CYP2D6*31 | CYP2D6.31 | 1661GC; | M20 | R296C; | . | . | Marez et |
| | | 2850CT; | | R440H; | | | al, 1997 |
| | | 4042GA; | | S486T | | | |
| | | 4180GC | | | | | |
| CYP2D6*32 | CYP2D6.32 | 1661GC; | M19 | R296C; | . | . | Marez et |
| | | 2850CT; | | E410K; | | | al, 1997 |
| | | 3853GA; | | S486T | | | |
| | | 4180GC | | | | | |
| CYP2D6*33 | CYP2D6.33 | 2483GT | CYP2D6*1C | A237S | Normal (s) | . | Marez et |
| | | | | | | | al, 1997 |
| CYP2D6*34 | CYP2D6.34 | 2850CT | CYP2D6*1D | R296C | . | . | Marez et |
| | | | | | | | al, 1997 |
| CYP2D6*35 | CYP2D6.35 | 31GA; | CYP2D6*2B | V11M; | Normal (s) | . | Marez et |
| | | 1661GC; | | R296C; | | | al, 1997 |
| | | 2850CT; | | S486T | | | |
| | | 4180GC | | | | | |
| CYP2D6*35X2 | CYP2D6.35 | 31GA; | . | V11M; | Incr | . | Griese et |
| | | 1661GC; | | R296C; | | | al, 1998 |
| | | 2850CT; | | S486T | | | |
| | | 4180GC | | | | | |
| CYP2D6*36 | CYP2D6.36 | 100CT; | CYP2D6C | P34S; | Decr | Decr | Wang, |
| | | 1039CT; | h2 | S486T | (d) | (b) | 1992 |
| | | 1661GC; | | | | | Johansson |
| | | 4180GC; | | | | | et al, 1994 |
| | | gene | | | | | Leathart et |
| | | conversion | | | | | al, 1998 |
| | | to CYP2D7 | | | | | |
| | | in exon 9 | | | | | |
| CYP2D6*37 | CYP2D6.37 | 100CT; | CYP2D6* | P34S; | . | . | Marez et |
| | | 1039CT; | 10D | R201H; | | | al, 1997 |
| | | 1661GC; | | S486T | | | |
| | | 1943GA; | | | | | |
| | | 4180GC; | | | | | |
| CYP2D6*38 | . | 2587-2590 | N2 | Frameshift | None | . | Leathart et |
| | | delGA | | | | | al, 1998 |
| | | CT | | | | | |
| CYP2D6*39 | CYP2D6.39 | 1661GC; | | S486T | | | Submitted |
| | | 4180GC | | | | | 17-Aug-00 |
| | | | | | | | by Dr. T. |
| | | | | | | | Shimada |
| CYP2D6*40 | CYP2D6.40 | 1023CT; | | T107I; | None (dx) | | Submitted |
| | | 1661GC; | | 172-174 | | | 28-Feb-01 |
| | | 1863 | | (FRP)3; | | | by Dr. A. |
| | | ins(TTT | | R296C; | | | Gaedigk |
| | | CGC | | S486T | | | |
| | | CCC)2; | | | | | |
| | | 2850 CT; | | | | | |
| | | 4180GC | | | | | |
| CYP2D6*41 | CYP2D6.2 | −1235AG; | | R296C; | Decr (s) | | Raimundo |
| | | −740CT; | | S486T | | | et al, 2000 |
| | | −678GA; | | | | | This allele |
| | | 1661GC; | | | | | is being |
| | | 2850CT; | | | | | further |
| | | 4180GC | | | | | characterised. |

b, bufuralol;
d, debrisoquine;
dx, dextromethorphan;
s, sparteine
SwissProtGenBankOMIMGeneCards
Note:
The −1584CG; −1235AG; −740CT and −678GA polymorphisms are probably found in most alleles of the CYP2D6*2 series (Raimundo et al, 2000).

TABLE 18

| Gene | | | mRNA/ | Structural |
|---|---|---|---|---|
| (Synonyms) | Locus | Accession #s | Protein | Information |
| CYP4A11 | | NT_029224 | 2815 bp | |
| “cytochrome | | (working draft | 519 aa | |
| P450, | | chromo1) | | |
| subfamily IVA, | | | | |
| polypeptide 11” | | NM_000778 (m) | | |
| (CP4Y, | | NP_000769 (p) | | |
| CYP4A2, | | | | |
| CYP4AII) | | | | |

Allelic Variants from SNP Database:

| Contig | Contig | dbSNPrs# | Protein | dbSNP | Protein | Codon | Amino |
|---|---|---|---|---|---|---|---|
| Accession | Position | (Cluster ID) | Accession | Allele | Residue | Position | Acid |
| NT_029224 | 405284 | rs2056899 | XP_037166 | a | N | 1 | 48 |
| | | | | t | Y | | |
| NT_029224 | 405350 | rs2056900 | XP_037166 | g | G | 1 | 26 |
| | | | | a | S | | |

TABLE 19

| Gene | | | mRNA | Structural |
|---|---|---|---|---|
| (Synonyms) | Locus | Accession #s | Protein | Information |
| CYP4F2 | 19pter- | NT_011281 | 2360 bp | |
| “cytochrome | p13.11 | (working draft | 520 aa | |
| P450, | | chromo19) | | |
| subfamily IVF, | | NT_025130 | | |
| polypeptide 2” | | (working draft | | |
| (CPF2) | | chromo19) | | |
| | | U02388 (m) | | |
| | | NM_001082 (m) | | |
| | | NP_001073 (p) | | |

Allelic Variants from SNP Database:

| Contig | Contig | dbSNPrs# | Protein | dbSNP | Protein | Codon | Amino |
|---|---|---|---|---|---|---|---|
| Accession | Position | (Cluster ID) | Accession | Allele | Residue | Position | Acid |
| NT_011281 | 77228 | rs2108622 | XP_051256 | g | V | 1 | 433 |
| | | | | a | M | | |

TABLE 20

| Gene | | | mRNA/ | Structural |
|---|---|---|---|---|
| (Synonyms) | Locus | Accession #s | Protein | Information |
| CYP11A | 15q23- | D00169 | 1821 bp | |
| “cytochrome | q24 | | 521 aa | |
| P450, | | NT_010298 (g) | | |
| subfamily XIA | | M14565 (m) | | |
| (cholesterol | | NM_000781 (m) | | |
| side chain | | NP_000772 (p) | | |
| cleavage)” | | | | |
| (P450SCC, | | | | |
| cytochrome P4 | | | | |
| 50C11A1) | | | | |

Allelic Variants from SNP Database:

| Contig | Contig | dbSNPrs# | Protein | dbSNP | Protein | Codon | Amino |
|---|---|---|---|---|---|---|---|
| Accession | Position | (Cluster ID) | Accession | Allele | Residue | Position | Acid |
| NT_010298 | 262118 | rs1130841 | XP_007646 | g | C | 2 | 16 |
| | | | | a | Y | | |
| NT_010298 | 281261 | rs1049968 | XP_007646 | c | I | 3 | 301 |
| | | | | g | M | | |
| NT_010298 | 281298 | rs6161 | XP_007646 | g | E | 1 | 314 |
| | | | | a | K | | |
| NT_010298 | 281298 | rs6161 | XP_027406 | g | E | 1 | 4 |
| | | | | a | K | | |

TABLE 21

| Gene | | | mRNA/ | Structural |
|---|---|---|---|---|
| (Synonyms) | Locus | Accession #s | Protein | Information |
| CYP11B1 | 8q21 | D10169, D90428, | 2092 bp | |
| “cytochrome | | X55765 (exon1 and | 503 aa | |
| P450, | | 5′ flanking region) | | |
| subfamily XIB | | D16153 (exon 1 and | | |
| (steroid 11- | | 2 normal) | | |
| beta- | | M32863, J05140 | | |
| hydroxylase), | | (exon 1 and 2) | | |
| polypeptide 1” | | M32878 (exon 3-8) | | |
| (FHI, CPN1, | | D16154 (exon 3-9) | | |
| CYP11B, | | M32879 (exon 9) | | |
| P450C11) | | NT_008127 | | |
| | | (working draft | | |
| | | chromo8) | | |
| | | NT_008127 (g) | | |
| | | X55764 (m) | | |
| | | NM_000497 (m) | | |
| | | NP_000488 (p) | | |

Allelic Variants from Scientific Literature:

| | Protein | | |
|---|---|---|---|
| DNA Variant | Variant | Phenotype | References |
| ? | Pro42Ser | Steroid 11-Beta- | OMIM 202010 |
| | | hydroxylase | |
| | | deficiency | |
| ? | Thr319Met | Steroid 11-Beta- | OMIM 202010 |
| | | hydroxylase | |
| | | deficiency | |
| ? | Asn133His | Steroid 11-Beta- | OMIM 202010 |
| | | hydroxylase | |
| | | deficiency | |
| ? | Arg374Gln | Steroid 11-Beta- | OMIM 202010 |
| | | hydroxylase | |
| | | deficiency | |
| ? | Thr318Met | Steroid 11-Beta- | OMIM 202010 |
| | | hydroxylase | |
| | | deficiency | |
| CGC > CAC | Arg448His | Steroid 11-Beta- | OMIM 202010 |
| | | hydroxylase | |
| | | deficiency | |

Allelic Variants from SNP Database:

| Contig | Contig | dbSNPrs# | Protein | dbSNP | Protein | Codon | Amino |
|---|---|---|---|---|---|---|---|
| Accession | Position | (Cluster ID) | Accession | Allele | Residue | Position | Acid |
| NT_008127 | 147509 | rs5294 | XP_030748 | t | Y | 1 | 439 |
| | | | | c | H | | |
| NT_008127 | 147747 | rs4541 | XP_030748 | c | A | 2 | 386 |
| | | | | t | V | | |
| NT_008127 | 147756 | rs5312 | XP_030748 | a | E | 2 | 383 |
| | | | | t | V | | |
| NT_008127 | 148261 | rs6407 | XP_030748 | g | A | 1 | 348 |
| | | | | a | T | | |
| NT_008127 | 148788 | rs5292 | XP_030748 | c | L | 1 | 293 |
| | | | | g | V | | |
| NT_008127 | 148823 | rs5291 | XP_030748 | g | S | 2 | 281 |
| | | | | a | N | | |
| NT_008127 | 149180 | rs5288 | XP_030748 | t | F | 3 | 257 |
| | | | | g | L | | |
| NT_008127 | 149208 | rs4547 | XP_030748 | c | T | 2 | 248 |
| | | | | t | I | | |
| NT_008127 | 149286 | rs5308 | XP_030748 | a | N | 2 | 222 |
| | | | | c | T | | |
| NT_008127 | 149608 | rs5287 | XP_030748 | g | M | 3 | 160 |
| | | | c | I | | | |
| NT_008127 | 152097 | rs5282 | XP_030748 | g | D | 1 | 63 |
| | | | | c | H | | |
| NT_008127 | 152156 | rs4534 | XP_030748 | g | R | 2 | 43 |
| | | | | a | Q | | |
| NT_008127 | 152255 | rs6405 | XP_030748 | g | C | 2 | 10 |
| | | | | a | Y | | |

TABLE 22

| Gene | | | mRNA/ | Structural |
|---|---|---|---|---|
| (Synonyms) | Locus | Accession #s | Protein | Information |
| CYP11B2 | 8q21-q22 | D13752 | 2936 bp | |
| “cytochrome | | | 503 aa | |
| P450, | | X54741 (m) | | |
| subfamily XIB | | NM_000498 (m) | | |
| (steroid 11- | | NP_000489 (p) | | |
| beta- | | | | |
| hydroxylase), | | | | |
| polypeptide 2” | | | | |
| (CPN2, | | | | |
| CYP11B, | | | | |
| CYP11BL, P- | | | | |
| 450C18, | | | | |
| P450aldo) | | | | |

Allelic Variants from Scientific Literature:

| | Protein | | |
|---|---|---|---|
| DNA Variant | Variant | Phenotype | References |
| ? | Lys173Arg | Low renin, | OMIM 124080 |
| | | susceptibility to | |
| | | hypertension | |
| ? | Glu198Asp | Congenital | OMIM 124080 |
| | | hypoaldosteronism | |
| ? | Thr185Ile | Congenital | OMIM 124080 |
| | | hypoaldosteronism | |
| ? | Leu461Pro | Congenital | OMIM 124080 |
| | | hypoaldosteronism | |
| GTG > GCG | Val386Ala | Congenital | OMIM 124080 |
| | | hypoaldosteronism | |
| CGG > TGG | Arg181Trp | Congenital | OMIM 124080 |
| | | hypoaldosteronism | |

TABLE 23

| Gene | | | mRNA/ | Structural |
|---|---|---|---|---|
| (Synonyms) | Locus | Accession #s | Protein | Information |
| CYP17 | 10q24.3 | M19489 | 1755 bp | |
| “cytochrome | | | 508 aa | |
| P450, | | NT_029393 (g) | | |
| subfamily XVII | | M14564 (m) | | |
| (steroid 17- | | NM_000102 (m) | | |
| alpha- | | NP_000093 (p) | | |
| hydroxylase), | | | | |
| adrenal | | | | |
| hyperplasia” | | | | |
| (CPT7, S17AH, | | | | |
| P450C17) | | | | |

Allelic Variants from Scientific Literature:

| | Protein | | |
|---|---|---|---|
| DNA Variant | Variant | Phenotype | References |
| T > G | PHE417CYS | Alpha- | OMIM 202110 |
| | | hydroxylase/17,20- | |
| | | lyase deficiency | |
| G > A | ARG358GLN | Alpha- | OMIM 202110 |
| | | hydroxylase/17,20- | |
| | | lyase deficiency | |
| G > A | ARG347HIS | Alpha- | OMIM 202110 |
| | | hydroxylase/17,20- | |
| | | lyase deficiency | |
| CGG > TGG | ARG96TRP | Alpha- | OMIM 202110 |
| | | hydroxylase/17,20- | |
| | | lyase deficiency | |
| CCA > ACA | PRO342THR | Alpha- | OMIM 202110 |
| | | hydroxylase/17,20- | |
| | | lyase deficiency | |
| CGA > TGA | ARG239TER | Alpha- | OMIM 202110 |
| | | hydroxylase/17,20- | |
| | | lyase deficiency | |
| | SER106PRO | Adrenal hyperplasia | OMIM 202110 |

Allelic Variants from SNP Database:

| Contig | Contig | dbSNPrs# | Protein | dbSNP | Protein | Codon | Amino |
|---|---|---|---|---|---|---|---|
| Accession | Position | (Cluster ID) | Accession | Allele | Residue | Position | Acid |
| NT_029393 | 754865 | rs762563 | XP_005915 | c | C | 3 | 22 |
| | | | | g | W | | |

TABLE 24

| Gene | | | mRNA/ | Structural |
|---|---|---|---|---|
| (Synonyms) | Locus | Accession #s | Protein | Information |
| CYP19 | 15q21.1 | L21982 (gene, | 3007 and 3116 | |
| “cytochrome | | untranslated exon | bp | |
| P450, | | I.4) | 503 aa | |
| subfamily XIX | | NT_010204 | | |
| (aromatization | | (working draft | | |
| of androgens)” | | chromo15) | | |
| (ARO, ARO1, | | | | |
| CPV1, CYAR, | | NM_000103 (m) | | |
| P-450AROM) | | NM_031226 (m) | | |
| | | NP_000094 (p) | | |
| | | NP_112503 (p) | | |

Allelic Variants from Scientific Literature:

| | Protein | | |
|---|---|---|---|
| DNA Variant | Variant | Phenotype | References |
| C1303T | Arg435Cys | Aromatase | OMIM 107910 |
| | | deficiency | |
| G1310A | Cys437Tyr | Aromatase | OMIM 107910 |
| | | deficiency | |
| C1123T | Arg375Cys | Aromatase | OMIM 107910 |
| | | deficiency | |
| G1094A | Arg365Gln | Aromatase | OMIM 107910 |
| | | deficiency | |

Allelic Variants from SNP Database:

| Contig | Contig | dbSNPrs# | Protein | dbSNP | Protein | Codon | Amino |
|---|---|---|---|---|---|---|---|
| Accession | Position | (Cluster ID) | Accession | Allele | Residue | Position | Acid |
| NT_010204 | 1691214 | rs2236722 | XP_035593 | t | W | 1 | 39 |
| | | | | c | R | | |
| NT_010204 | 1706104 | rs1803154 | XP_035593 | a | K | 1 | 108 |
| | | | | t | * | | |
| NT_010204 | 1718241 | rs700519 | XP_035593 | c | R | 1 | 264 |
| | | | | t | C | | |

TABLE 25

| Gene | | | mRNA/ | Structural |
|---|---|---|---|---|
| (Synonyms) | Locus | Accession #s | Protein | Information |
| CYP21A2 | 6p21.3 | M13936 | 2112 bp | |
| “cytochrome | | | 495 aa | |
| P450, | | NG_000013 (g) | | |
| subfamily XXIA | | NT_007592 (g) | | |
| (steroid 21- | | NM_000500 (m) | | |
| hydroxylase, | | M26856 (m) | | |
| congenital | | NP_000491 (p) | | |
| adrenal | | | | |
| hyperplasia), | | | | |
| polypeptide 2” | | | | |
| (CPS1, | | | | |
| CA21H, | | | | |
| CYP21, | | | | |
| CYP21B, | | | | |
| P450C21B, | | | | |
| P450c21B) | | | | |

Allelic Variants from Scientific Literature:

| | Protein | | |
|---|---|---|---|
| DNA Variant | Variant | Phenotype | References |
| | Gly424Ser | Adrenal Hyperplasia | OMIM 201910 |
| | Glu380Asp | Adrenal Hyperplasia | OMIM 201910 |
| | Arg339His | Adrenal Hyperplasia | OMIM 201910 |
| | Met238Lys | Adrenal Hyperplasia | OMIM 201910 |
| | Val236Glu | Adrenal Hyperplasia | OMIM 201910 |
| | Ile235Asn | Adrenal Hyperplasia | OMIM 201910 |
| | Tyr102Arg | 21-Hydroxylase | OMIM 201910 |
| | | polymorphism | |
| | Pro453Ser | Adrenal Hyperplasia | OMIM 201910 |
| | Gly292Ser | Adrenal Hyperplasia | OMIM 201910 |
| | Ser268Thr | 21-Hydroxylase | OMIM 201910 |
| | | polymorphism | |
| | Pro30Leu | Adrenal Hyperplasia | OMIM 201910 |
| | Arg356Trp | Adrenal Hyperplasia | OMIM 201910 |
| | Val281Leu | Adrenal Hyperplasia | OMIM 201910 |
| | Ile172Asn | Adrenal Hyperplasia | OMIM 201910 |

Allelic Variants from SNP Database:

| Contig | Contig | dbSNPrs# | Protein | dbSNP | Protein | Codon | Amino |
|---|---|---|---|---|---|---|---|
| Accession | Position | (Cluster ID) | Accession | Allele | Residue | Position | Acid |
| NT_007592 | 8200835 | rs6473 | XP_004200 | g | S | 2 | 494 |
| | | | | a | N | | |
| NT_007592 | 8200956 | rs6445 | XP_004200 | c | P | 1 | 454 |
| | | | | t | S | | |
| NT_007592 | 8201852 | rs6471 | XP_004200 | g | V | 1 | 282 |
| | | | | t | L | | |
| NT_007592 | 8201890 | rs6472 | XP_004200 | g | S | 2 | 269 |
| | | | | c | T | | |
| NT_007592 | 8202146 | rs6476 | XP_004200 | t | M | 2 | 240 |
| | | | | a | K | | |
| NT_007592 | 8202414 | rs1040310 | XP_004200 | c | D | 3 | 184 |
| | | | | g | E | | |
| NT_007592 | 8202536 | rs6475 | XP_004200 | t | I | 2 | 173 |
| | | | | a | N | | |
| NT_007592 | 8202853 | rs6474 | XP_004200 | g | R | 2 | 103 |
| | | | | a | K | | |
| NT_007592 | 8200835 | rs6473 | XP_042400 | g | S | 2 | 225 |
| | | | | a | N | | |
| NT_007592 | 8200956 | rs6445 | XP_042400 | c | P | 1 | 185 |
| | | | | t | S | | |
| NT_007592 | 8201852 | rs6471 | XP_042400 | g | V | 1 | 13 |
| | | | | t | L | | |

TABLE 26

| Gene | | | mRNA/ | Structural |
|---|---|---|---|---|
| (Synonyms) | Locus | Accession #s | Protein | Information |
| CYP27A1 | 2q33-qter | S62709 (5′ | 2059 bp | Cali et al. |
| “cytochrome | | region) | 531 aa | (1991) |
| P450, | | NT_005289 | | OMIM |
| subfamily | | (working draft | | (213700 One |
| XXVIIA (steroid | | chromo2) | | mutation, |
| | | | | called by them |
| 27- | | NM_000784 | | CTX1, was at |
| hydroxylase, | | (m) | | codon 446 near |
| cerebrotendinous | | NP_000775 | | the heme |
| xanthomatosis), | | (p) | | ligand, cys444. |
| polypeptide 1” | | | | The second, |
| (CTX, CP27, | | | | called CTX2, |
| CYP27) | | | | was at codon |
| | | | | 362 in the |
| | | | | adrenodoxin |
| | | | | binding region. |

Allelic Variants from Scientific Literature:

| | Protein | | |
|---|---|---|---|
| DNA Variant | Variant | Phenotype | References |
| G > A | Arg372Gln | Cerebrotendinous | OMIM 213700 |
| | | xanthomatosis | |
| C > T | Arg441Trp | Cerebrotendinous | OMIM 213700 |
| | | xanthomatosis | |
| G-to-A | Arg441Gln | Cerebrotendinous | OMIM 213700 |
| | | xanthomatosis | |
| (CGPy) to | Arg362Cys | Cerebrotendinous | OMIM 213700 |
| cysteine | | xanthomatosis | |
| codons | | | |
| (TGPy). | | | |
| (CGPy) to | Arg446Cys | Cerebrotendinous | OMIM 213700 |
| cysteine | | xanthomatosis | |
| codons | | | |
| (TGPy). | | | |

TABLE 27

| Gene | | | mRNA/ | Structural |
|---|---|---|---|---|
| (Synonyms) | Locus | Accession #s | Protein | Information |
| CYP51 | 7q21.2- | AH006655 | 3381 bp | |
| “cytochrome | q21.3 | | | |
| P450, 51 | | NT_029333 (g) | | |
| (lanosterol 14- | | NM_000786 (m) | | |
| alpha- | | NP_000777 (p) | | |
| demethylase)” | | | | |
| (LDM, CP51, | | | | |
| CYPL1, | | | | |
| P450L1, P450- | | | | |
| 14DM) | | | | |

Allelic Variants from SNP Database:

| Contig | Contig | dbSNPrs# | Protein | dbSNP | Protein | Codon | Amino |
|---|---|---|---|---|---|---|---|
| Accession | Position | (Cluster ID) | Accession | Allele | Residue | Position | Acid |
| NT_029333 | 2609660 | rs2229188 | XP_004663 | t | V | 2 | 19 |
| | | | | c | A | | |

TABLE 28

| Gene | | | mRNA/ | Structural |
|---|---|---|---|---|
| (Synonyms) | Locus | Accession #s | Protein | Information |
| EPHX1 | 1q42.1 | AF253417, L29766, | 1856 bp | |
| “epoxide | | L25880 | 455 aa | |
| hydrolase 1, | | | | |
| microsomal | | NT_004525 (g) | | |
| (xenobiotic)” | | NM_000120 (m) | | |
| (MEH, EPHX) | | NP_000111 (p) | | |

Allelic Variants from Scientific Literature:

| | Protein | | |
|---|---|---|---|
| DNA Variant | Variant | Phenotype | References |
| ? | Tyr113His | Epoxide hydrolase | OMIM 132810 |
| | | polymorphism, | |
| | | susceptibility to | |
| | | aflatoxin B1? | |

Allelic Variants from SNP Database:

| Contig | Contig | dbSNPrs# | Protein | dbSNP | Protein | Codon | Amino |
|---|---|---|---|---|---|---|---|
| Accession | Position | (Cluster ID) | Accession | Allele | Residue | Position | Acid |
| NT_004525 | 1595032 | rs2234701 | XP_001799 | g | R | 2 | 454 |
| | | | | a | Q | | |
| NT_004525 | 1595753 | rs2137841 | XP_001799 | t | H | 3 | 387 |
| | | | | a | Q | | |
| NT_004525 | 1601658 | rs2234922 | XP_001799 | g | R | 2 | 139 |
| | | | | a | H | | |
| NT_004525 | 1608433 | rs1051740 | XP_001799 | c | H | 1 | 113 |
| | | | | t | Y | | |
| NT_004525 | 1611484 | rs2234697 | XP_001799 | c | R | 1 | 49 |
| | | | | t | C | | |

TABLE 29

| Gene | | | mRNA/ | Structural |
|---|---|---|---|---|
| (Synonyms) | Locus | Accession #s | Protein | Information |
| EPHX2 | 8p21-p12 | X97024 (exon 1) | 2100 bp | |
| (epoxide | | X97038 (exon 17, | 554 aa | |
| hydrolase 2, | | 18 and 19) | | |
| cytoplas) | | NT_007988 | | |
| | | (working draft | | |
| | | chromo 8) | | |
| | | NM_001979 (m) | | |
| | | L05779 (m) | | |
| | | NP_001970 (p) | | |

Allelic Variants from SNP Database:

| Contig | Contig | dbSNPrs# | Protein | dbSNP | Protein | Codon | Amino |
|---|---|---|---|---|---|---|---|
| Accession | Position | (Cluster ID) | Accession | Allele | Residue | Position | Acid |
| NT_007988 | 233832 | rs751141 | XP_005114 | g | R | 2 | 287 |
| | | | | a | Q | | |

TABLE 30

| Gene | | | mRNA/ | Structural |
|---|---|---|---|---|
| (Synonyms) | Locus | Accession #s | Protein | Information |
| GUSB | 7q21.11 | M65002 (5′ end) | 2191 bp | |
| (glucuronidase, | | Pseudogene | 651 aa | |
| beta) | | AL021368 (BAC | | |
| | | 55C20 on chromo6) | | |
| | | M15182 (m) | | |
| | | NM_000181 (m) | | |
| | | NP_000172 (p) | | |

Allelic Variants from Scientific Literature:

| | Protein | | |
|---|---|---|---|
| DNA Variant | Variant | Phenotype | References |
| ? | Trp446Ter | Mucopolysaccharidosis | |
| ? | Trp507Ter | Mucopolysaccharidosis | |
| ? | Tyr495Cys | Mucopolysaccharidosis | |
| ? | Pro148Ser | Mucopolysaccharidosis | |
| C1831T | Arg611Trp | Mucopolysaccharidosis | |
| C1061T | Arg354Val | Mucopolysaccharidosis | |
| C672T | Arg216Trp | Mucopolysaccharidosis | |
| C > T | Arg382Cys | Mucopolysaccharidosis | |
| C > T | Ala619Val | Mucopolysaccharidosis | |

TABLE 31

| Gene | | | mRNA/ | Structural |
|---|---|---|---|---|
| (Synonyms) | Locus | Accession #s | Protein | Information |
| KCNH2 | 7q35-q36 | NT_007704 | 4070 bp | |
| “potassium | | (working draft | 1159 aa | |
| voltage-gated | | chromo7) | | |
| channel, | | | | |
| subfamily H | | U04270 (m) | | |
| (eag-related), | | NP_000229 (p) | | |
| member 2” | | | | |
| (HERG, LQT2) | | | | |

Allelic Variants from Scientific Literature:

| | Protein | | |
|---|---|---|---|
| DNA Variant | Variant | Phenotype | References |
| G1468A | Ala490Thr | Long QT syndrome | OMIM 152427 |
| ? | Gly572Arg | Long QT syndrome | OMIM 152427 |
| ? | Arg582Cys | Long QT syndrome | OMIM 152427 |
| G1882A | Gly628Ser | Long QT syndrome | OMIM 152427 |
| G2647A | Val822Met | Long QT syndrome | OMIM 152427 |
| T1961G | Ile593Arg | Long QT syndrome | OMIM 152427 |
| A1408G | Asn470Asp | Long QT syndrome | OMIM 152427 |
| C1682T | Ala561Val | Long QT syndrome | OMIM 152427 |

Allelic Variants from SNP Database:

| Contig | Contig | dbSNPrs# | Protein | dbSNP | Protein | Codon | Amino |
|---|---|---|---|---|---|---|---|
| Accession | Position | (Cluster ID) | Accession | Allele | Residue | Position | Acid |
| NT_007704 | 8393 | rs731506 | XP_004743 | t | V | 2 | 41 |
| | | | | g | G | | |

TABLE 32

| Gene | | | mRNA/ | Structural |
|---|---|---|---|---|
| (Synonyms) | Locus | Accession #s | Protein | Information |
| LTA4H | 12q22 | U27293 (exon 19 | 2060 bp | |
| “leukotriene A4 | | and complete cds.) | 611 aa | |
| hydrolase” | | | | |
| | | NM_000895 (m) | | |
| | | J03459 (m) | | |
| | | NP_000886 (p) | | |

Allelic Variants from SNP Database:

| Contig | Contig | dbSNPrs# | Protein | dbSNP | Protein | Codon | Amino |
|---|---|---|---|---|---|---|---|
| Accession | Position | (Cluster ID) | Accession | Allele | Residue | Position | Acid |
| NT_009685 | 277202 | rs1803916 | XP_012237 | c | T | 2 | 600 |
| | | | | g | S | | |

TABLE 33

| Gene | | | mRNA/ | Structural |
|---|---|---|---|---|
| (Synonyms) | Locus | Accession #s | Protein | Information |
| PTGIS | 20q13.11-q13.13 | D83393 (exon 1) | 5605 bp | |
| “prostaglandinl2 | | NT_011362 | 500 aa | |
| | | (working draft | | |
| (prostacyclin) | | chromo20) | | |
| synthase” | | | | |
| (CYP8, PGIS, | | NP_000952 (m) | | |
| PTGI, | | NP_000952 (p) | | |
| CYP8A1) | | | | |

Allelic Variants from SNP Database:

| Contig | Contig | dbSNPrs# | Protein | dbSNP | Protein | Codon | Amino |
|---|---|---|---|---|---|---|---|
| Accession | Position | (Cluster ID) | Accession | Allele | Residue | Position | Acid |
| NT_011362 | 13177081 | rs5584 | XP_030507 | c | P | 1 | 500 |
| | | | | t | S | | |
| NT_011362 | 13193363 | rs5626 | XP_030507 | c | R | 1 | 236 |
| | | | | t | C | | |
| NT_011362 | 13213471 | rs5624 | XP_030507 | t | F | 1 | 171 |
| | | | | c | L | | |
| NT_011362 | 13213521 | rs5623 | XP_030507 | a | E | 2 | 154 |
| | | | | c | A | | |
| NT_011362 | 13217020 | rs5622 | XP_030507 | t | S | 3 | 118 |
| | | | | a | R | | |

TABLE 34

| Gene | | | mRNA/ | Structural |
|---|---|---|---|---|
| (Synonyms) | Locus | Accession #s | Protein | Information |
| TPMT | 6p22.3 | U81562 | 2742 bp | |
| “thiopurine S- | | NT_007180 (g) | 245 aa | |
| methyltransferase” | | NM_000367 (m) | | |
| | | S62904 (m) | | |
| | | NP_000358 (p) | | |

Allelic Variants from Scientific Literature:

| | Protein | | |
|---|---|---|---|
| DNA Variant | Variant | Phenotype | References |
| A719G | Tyr240Cys | 6-mercaptopurine | OMIM 187680 |
| | | sensitivity | |
| G644A | Arg215His | 6-mercaptopurine | OMIM 187680 |
| | | sensitivity | |
| G460A | Ala154Thr | 6-mercaptopurine | OMIM 187680 |
| | | sensitivity | |
| G238C | Ala80Pro | 6-mercaptopurine | OMIM 187680 |
| | | sensitivity | |

Allelic Variants from SNP Database:

| Contig | Contig | dbSNPrs# | Protein | dbSNP | Protein | Codon | Amino |
|---|---|---|---|---|---|---|---|
| Accession | Position | (Cluster ID) | Accession | Allele | Residue | Position | Acid |
| NT_007180 | 151037 | rs1800462 | XP_012752 | g | A | 1 | 80 |
| | | | | c | P | | |
| NT_007180 | 164074 | rs1142345 | XP_012752 | a | Y | 2 | 240 |
| | | | | g | C | | |

### TABLE REFERENCES

Cascorbi et al., Clin. Pharmacol. Ther. 69:169-174 (2001)

Choi et al., Cell 53:519-529 (1988)

Hoffmeyer et al., Proc. Natl. Acad. Sci. USA 97:3473-3478 (2000)

Ito et al., Pharmacogenetics 11:175-184 (2001)

Mickley et al., Blood 91:1749-1756 (1998)

Safa et al., Proc. Natl. Acad. Sci. USA 87:7225-7229 (1990)

Tanabe et al., J. Pharmacol. Exp. Ther. 297:1137-1143 (2001)

Toh et al., Amer. J. Hum. Genet. 64:739-746 (1999)

Wada et al., Hum. Mol. Genet. 7:203-207(1998)

TABLE 35
BCR-ABL Kinase Domain Mutations
Affecting Response to Imatinib

| | | Proposed mechanism |
|---|---|---|
| Mutation | Phase of disease* | of resistance |
| M244V | CP | impairs |
| | | conformational |
| | | change (P loop) |
| G250E | MBC | impairs |
| | | conformational |
| | | change (P loop) |
| Q252H/R | MBC | impairs |
| | | conformational |
| | | change (P loop) |
| Y253F/H | MBC, LBC | impairs |
| | | conformational |
| | | change (P loop) |
| E255K | MBC, LBC, CP, P-MBC | impairs |
| | | conformational |
| | | change (P loop) |
| T315I | MBC, LBC, CP, P-MBC | directly affects |
| | | imatinib binding |
| F317L | MBC, CP | directly affects |
| | | imatinib binding |
| M351T | MBC, LBC, CP | impairs |
| | | conformational |
| | | change (adjacent to |
| | | activation loop) |
| E355G | MBC | impairs |
| | | conformational |
| | | change (adjacent to |
| | | activation loop) |
| F359V | MBC, CP | directly affects |
| | | imatinib binding |
| V379I | CP-NCR | impairs |
| | | conformational |
| | | change |
| | | (activation |
| | | loop) |
| L387M | CP | impairs |
| | | conformational |
| | | change |
| | | (activation |
| | | loop) |
| H396R | MBC, CP | impairs |
| | | conformational |
| | | change |
| | | (activation |
| | | loop) |

*MBC: meyloid blast crisis
LBC: lyjphoid blast crisis
CP: chronic phase
CP-NCR: chronic phase with hematologic response in the absence of cytogenetic response (cytogenetic nonresponder)

TABLE 36
Beta tubulin (Isoform M40) Mutations
Affecting Response to Paclitaxel

| DNA Variant | Protein Variant | Phenotype | Reference |
|---|---|---|---|
| T810G | Phe270Val | paclitaxel | Gianakakou et al., J. Biol. |
| | | resistance | Chem. 272: 17118-25 |
| | | | (1997) |
| G1092A | Ala364Thr | paclitaxel | Gianakakou et al., J. Biol. |
| | | resistance | Chem. 272: 17118-25 |
| | | | (1997) |

The collections of the present invention require that the genotypically distinct coisogenic cells be in sufficient spatial proximity to one another as readily and contemporaneously to be subject to a common experimental protocol, yet remain separately assayable.

Separate assayability can easily be effected by maintaining each of the genotypically distinct coisogenic cells of the collection in fluid noncommunication with the others of the cells of the collection. Spatial proximity can be effected by disposing the cells within wells or other types of fluidly noncommunicating locations that are within or upon a common structure.

For example, each genotypically distinct cell (typically, cell line) can be disposed in a well (or wells) of a microtiter plate distinct from the well (or wells) in which genotypically-distinct cells are placed. Microtiter plates are now readily available commercially that have 24, 96, 384, 864, 1536, 3456, 6144, and 9600 wells. And variants abound. For example, U.S. Pat. No. 6,171,780 B1 describes low fluorescence multiwell platforms for cellular screening assays. U.S. Pat. No. 6,103,479 describes methods apparatus for non-uniform micro-patterned arrays of cells. Chiu et al., Proc. Natl. Acad. Sci. USA 97(6):2408-13 (2000) describe the patterned deposition of cells onto surfaces by using three-dimensional microfluidic systems. A wide variety of “chip-based”, microfluidic devices for arraying cells are also now described. See, e.g., U.S. Pat. No. 6,086,740 (“Multiplexed microfluidic devices and systems”).

Alternatively, the genotypically distinct cells of the collection can be maintained in fluid noncommunication by disposing each genotypically distinct cell (typically, as a genotypically distinct cell line) in a separate structurally discrete, fluidly noncommunicating container, such as a vial, ampule, or tube; spatial proximity can in such cases be effected by packaging the separate containers together. In such cases, the cell collections of the present invention take the form of a kit, and it is therefore another aspect of the present invention to provide kits comprising the coisogenic cell collections of the present invention.

The kits comprise at least five genotypically distinct cells, the cells contained within separate, structurally discrete, fluidly noncommunicating containers; the at least five structurally discrete containers are packaged together. As described above, each of the at least 5 genotypically distinct cells is coisogenic with respect the others of the at least 5 genotypically distinct cells at a target locus common thereamong.

Since the cell collections of the present invention can include a great many more than five genotypically distinct cells, the kits of the present invention can usefully and additionally include computer-readable media having at least one dataset that defines the genotype of the cells of the collection at least at the target locus; the dataset can usefully include links to extrinsic databases, such as the Online Mendelian Inheritance of Man (OMIM) (http://www.ncbi.nlm.nih.gov:80/entrez/query.fcgi?db=OM IM)), the Human Gene Mutation Database (HGMD) (http://archive.uwcm.ac.uk/uwcm/mg/hgmd0.html), or more general databases, such as GenBank, or the UCSC human genome project working draft (http://genome.ucsc.edu/).

Fluid noncommunication is not required where the genotypically distinct cells can be distinguished even in admixture. In such case, the cells can be contained in a common container, such as a tube, ampule, well, or dish; the required spatial proximity is of course thus necessarily maintained.

For example, if the assay measures cell proliferation under a chosen condition, such as exposure to a chemotherapeutic agent, e.g. paclitaxel or a derivative thereof, and the cells are individually bar coded, the cells can be commonly cultured in the presence of the drug agent, and the degree of individual proliferation assessed by stoichiometric amplification and quantification of their respective bar codes. See, e.g., U.S. Pat. No. 6,046,002, incorporated herein by reference in its entirety.

Additionally, the coisogenic cell collections of the present invention need not be in a form that can immediately be assayed. Rather, the collections can be provided in any physical form that will, at some point, permit the genotypically distinct cultured cells separately to be assayed. In one embodiment, for example, the cells can be provided frozen, either in individual tubes or ampules or collectively in the wells of a microtiter dish, thereafter to be thawed, propagated, and assayed. Where the cells are yeast cells, the cells can conveniently be provided frozen or lyophilized.

The invention further provides, in another aspect, methods of making the coisogenic cell collections of the present invention.

In a basic embodiment, the method comprises collecting at least 5 genotypically distinct cells, each of the cells being coisogenic with respect to the others of the at least 5 genotypically distinct cells at a target locus common thereamong, into a collection in which each of the genotypically distinct cells can be separately assayed.

Typically, but not invariably, the method further comprises the earlier step of making cells that are coisogenic at a common target locus. The coisogenic cells are made by engineering, into at least four of at least five cultured cells, the cells derived from a common eukaryotic ancestor cell, a genomic sequence alteration at a target locus common thereamong; the sequence alterations must be sufficient to cause at least five distinct protein sequences collectively to be encoded by the cells at the common target locus.

The genomic sequence alterations can be created by any means that permits mutations to be targeted to genomic sequence. In a presently preferred approach, mutations are targeted to a common target locus using modified single-stranded oligonucleotides (“targeting oligonucleotides”).

We have recently described methods for targeting single nucleotide changes directly into long pieces of genomic DNA present within YACs, BACs, and even intact cellular chromosomes through use of sequence-altering oligonucleotides. See international patent publication nos. WO 01/73002, WO 01/92512, and WO 02/10364; and commonly owned and copending U.S. provisional patent application No. 60/326,041, filed Sep. 27, 2001, No. 60/337,129, filed Dec. 4, 2001, No. 60/393,330, filed Jul. 1, 2002, No. 60/363,341, filed Mar. 7, 2002; No. 60/363,053, filed Mar. 7, 2002, and No. 60/363,054, filed Mar. 7, 2002, the disclosures of which are incorporated herein by reference in their entireties. These methods, described in further detail below, are presently preferred.

Other approaches for targeting sequence changes using sequence altering oligonucleotides have also been described. See e.g. U.S. Pat. Nos. 6,303,376; 5,776,744; 6,200,812; 6,074,853; 5,948,653; 6,136,601; 6,010,907; 5,888,983; 5,871,984; 5,760,012; 5,756,325; and 5,565,350, the disclosures of which are incorporated herein by reference in their entireties. These latter approaches typically have lower efficiency and are at present less preferred, although they may at times be used.

Changes can be targeted directly into cellular chromosomes within cultured eukaryotic cells. In other embodiments, changes can instead be targeted to recombinant constructs in vitro, with the modified target thereafter used to integrate the desired change into a cultured eukaryotic cell.

The first of these approaches is particularly preferred for creating coisogenic cell collections that are legacy-free, and/or exceptionally or perfectly coisogenic. The second approach is preferred, inter alia, in construction of coisogenic cell collections having identical targeted changes superimposed on different genetic backgrounds.

In the latter approach, the vector is usefully an artificial chromosome, such as YACs (yeast artificial chromosomes), BACs (bacterial artificial chromosomes), PACs (P-1 derived artificial chromosomes), HACs (human artificial chromosomes), and PLACs (plant artificial chromosomes).

Artificial chromosomes are reviewed in Larin et al., Trends Genet. 18(6):313-9 (2002); Choi et al., Methods Mol. Biol. 175:57-68 (2001); Brune et al., Trends Genet. 16(6):254-9 (2001); Ascenzioni et al., Cancer Lett. 118(2):135-42 (1997); Fabb et al., Mol. Cell. Biol. Hum. Dis. Ser. 5:104-24 (1995); Huxley, Gene Ther. 1 (1):7-12 (1994), the disclosures of which are incorporated herein by reference in their entireties. Other vectors that may be used include viral, typically eukaryotic viral, vectors, such as adenoviral, varicella, and herpesvirus vectors.

Yeast artificial chromosomes (YACs) are additionally described in Burke et al., Science 236:806; Peterson et al., Trends Genet. 13:61 (1997); Choi et al., Nature Genet., 4:117-223 (1993); Davies et al., Biotechnology 11:911-914 (1993); Matsuura et al., Hum. Mol. Genet., 5:451-459 (1996); Peterson et al., Proc. Natl. Acad. Sci., 93:6605-6609 (1996); and Schedl et al., Cell, 86:71-82 (1996)). Human artificial chromosomes (HACs) are additionally described in Kuroiwa et al., Nature Biotechnol. 18(10):1086-90 (2000); Henning et al., Proc. Natl. Acad. Sci. USA 96(2):592-7 (1999); Harrington et al., Nature Genet. 15(4):345-55 (1997). Bacterial artificial chromosomes (BACs) and P-1 derived artificial chromosomes (PACs) are further described in Mejia et al., Genome Res. 7:179-186 (1997); Shizuya et al., Proc. Natl. Acad. Sci. 89:8794-8797 (1992); Ioannou et al., Nature Genet., 6:84-89 (1994); Hosoda et al., Nucleic Acids Res. 18:3863 (1990). Other vectors useful in the present invention are further described in Sternberg et al., Proc. Natl. Acad. Sci. USA 87:103-107 (1990).

BACs have been developed for transformation of plants with high-molecular weight DNA using the T-DNA system (Hamilton, Gene 24:107-116 (1997); Frary et al., Transgenic Res. 10: 121-132 (2001)).

In certain useful embodiments, genomic targets are present within vectors that permit integration of the target into a cellular chromosome. In particularly useful embodiments, genomic targets are present within vectors that permit site-directed integration of the target into a cellular chromosome. Usefully, the vector is an artificial chromosome and site-specific integration may be performed by recombinase mediated cassette exchange (RMCE).

In RMCE, a region of DNA (cassette) desired to be integrated into a specific cellular chromosomal location is flanked in a recombinant vector by sites that are recognized by a site-specific recombinase, such as loxP sites and derivatives thereof for Cre recombinase and FRT sites and derivatives thereof for Flp recombinase. Other site-specific recombinases having cognate recognition/recombination sites useful in such methods are known (see, e.g., Blake et al., Mol. Microbiol. 23(2):387-98 (1997)).

The site in the cellular chromosome into which the cassette is desired site-specifically to be integrated is analogously flanked by recognition sites for the same recombinase.

To favor a double-reciprocal crossover exchange reaction between vector and chromosome, two approaches are typical. In the first, the two sites (such as lox or FRT) that flank the cassettes in both vector and cellular chromosome are heterospecific: that is, they differ from one another and recombine with each other with far lower efficiency than with sites identical to themselves. In the second, the lox or FRT sites are inverted. See, e.g., Baer et al., Curr. Opin. Biotechnol. 12:473-480 (2001); Langer et al., Nucl. Acids Res. 30:3067-3077 (2002); Feng et al., J. Mol. Biol. 292:779-785 (1999), the disclosures of which are incorporated herein by reference in their entireties.

Recombinational exchange of the cassettes from vector to cellular chromosome, with integration of the construct cassette site-specifically into the cellular chromosome, is effected by introducing the recombinant construct into the cell and expressing the site-specific recombinase appropriate to the recombination sites used. The site-specific recombinase may be expressed transiently or continuously, either from an episome or from a construct integrated into cellular chromosome, using techniques well known in the art.

Site-specific recombinational insertion provides a single-copy integrant of defined and chosen sequence in a defined cellular genomic milieu. It is known that such site-specific integration provides more consistent expression than does random integration. Feng et al., J. Mol. Biol. 292:779-285 (1999).

Our presently preferred methods for targeting single nucleotide changes directly into genomic DNA—whether targeted directly into a eukaryotic chromosome or first targeted into a recombinant construct in vitro—are further described in international patent publication nos. WO 01/73002, WO 01/92512, and WO 02/10364; and commonly owned and copending U.S. provisional patent application No. 60/326,041, filed Sep. 27, 2001, No. 60/337,129, filed Dec. 4, 2001, No. 60/393,330, filed Jul. 1, 2002, No. 60/363,341, filed Mar. 7, 2002; No. 60/363,053, filed Mar. 7, 2002, and No. 60/363,054, filed Mar. 7, 2002; the disclosures of which are incorporated herein by reference in their entireties.

Briefly, the method comprises combining the targeted nucleic acid, in the presence of cellular repair proteins, with a single-stranded oligonucleotide 17-121 nucleotides in length, the oligonucleotide having an internally unduplexed domain of at least 8 contiguous deoxyribonucleotides. The oligonucleotide is fully complementary in sequence to the sequence of a first strand of the nucleic acid target, but for one or more mismatches as between the sequences of the internally unduplexed deoxyribonucleotide domain and its complement on the target nucleic acid first strand. Each of the mismatches is positioned at least 8 nucleotides from each of the oligonucleotide's 5′ and 3′ termini, and the oligonucleotide has at least one terminal modification.

The oligonucleotide terminal modification is typically selected from the group consisting of at least one terminal locked nucleic acid (LNA), at least one terminal 2′—O—Me base analog, and at least three terminal phosphorothioate linkages.

LNAs are bicyclic and tricyclic nucleoside and nucleotide analogs and the oligonucleotides that contain such analogs. The basic structural and functional characteristics of LNAs and related analogues that usefully may be incorporated into the second (“annealing”) oligonucleotide in the methods of the present invention are disclosed in various publications and patents, including WO 99/14226, WO 00/56748, WO 00/66604, WO 98/39352, U.S. Pat. No. 6,043,060, and U.S. Pat. No. 6,268,490, the disclosures of which are incorporated herein by reference in their entireties. See also Singh et al., Chem. Commun. 1998: 455; Koshkin et al., Tetrahedron 54:3607 (1998); Koshkin et al., Tetrahedron Lett. 39:4381 (1998); Singh et al., Chem. Commun. 1998:1247, and are reviewed in Orum et al., “Locked nucleic acids: a promising molecular family for gene-function analysis and antisense drug development,” Curr. Opin. Mol. Ther. 3(3):239-43 (2001), the disclosures of which are incorporated herein by reference in their entireties.

Synthesis of LNA nucleosides and nucleoside analogs and oligonucleotides that contain them may be performed as disclosed in WO 99/14226, WO 00/56748, WO 00/66604, WO 98/39352, U.S. Pat. No. 6,043,060, and U.S. Pat. No. 6,268,490. Many may now be ordered commercially (Exiqon, Inc., Vedbaek, Denmark; Proligo LLC, Boulder, Colo., USA).

The oligonucleotides are typically at least 17 nucleotides in length, and can usefully be up to about 121 nucleotides in length, and even longer, although targeting oligonucleotides of about 17 to about 74 nucleotides in length are at present preferred. The oligonucleotides used to create the coisogenic cell collections may thus have lengths of 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, or 121 nt.

At present most preferred are targeting oligonucleotides at least about 25 bases in length, unless there are self-dimerization structures within the oligonucleotide; if the oligonucleotide has such an unfavorable structure, lengths longer than 35 bases are preferred.

The internally unduplexed alteration domain of the targeting oligonucleotide is preferably fully complementary to one strand of the target locus, except for the mismatched base (or up to about 3 mismatched bases) introduced to effect the gene alteration or conversion events. The central alteration domain is generally at least 8 nucleotides in length. Although it is presently preferred to locate the alteration domain approximately in the middle of the targeting oligonucleotide, there is no strict requirement for symmetrical extension adjacent to the alteration DNA domain. However, the base(s) targeted for alteration in the most preferred embodiments are at least about 8, 9 or 10 bases from each of the ends of the targeting oligonucleotide.

The targeting oligonucleotide preferably binds to the non-transcribed strand of a genomic DNA duplex.

The oligonucleotides used to make the coisogenic cell collections of the present invention preferably contain more than one of the aforementioned modifications (“backbone modifications”), preferably (but not obligately) at both ends of the oligonucleotide. In some embodiments, the backbone modifications are adjacent to one another. For oligonucleotides of the invention that are longer than about 17 to about 25 bases in length, internal as well as terminal region segments of the backbone can be altered.

The optimal number and placement of backbone modifications for any individual oligonucleotide will vary with the length of the oligonucleotide and the particular type of backbone modification(s) that are used, and may be determined by routine comparative studies, as further described in WO 01/73002 and commonly owned and copending U.S. patent application Ser. No. 09/818,875, filed Mar. 27, 2001, the disclosures of which are incorporated herein by reference in their entireties.

The sequence-altering oligonucleotide can be contacted to its genomic target within intact cells, within cell-free protein extracts having cellular repair proteins, or within purified protein fractions having cellular repair proteins.

Efficiency of conversion is defined herein as the percentage of recovered substrate molecules that have undergone a conversion event. Depending on the nature of the target genetic material, e.g. the genome of a cell or a genomic construct in a replicable vector, efficiency can be represented as the proportion of cells or clones containing an extrachromosomal element that exhibit a particular phenotype. Alternatively, representative samples of the target genetic material can be sequenced to determine the percentage that have acquired the desire change.

Efficiency can be increased using the methods set forth in commonly owned and copending U.S. provisional application serial No. 60/363,341, filed Mar. 7, 2002; No. 60/363,053, filed Mar. 7, 2002; and No. 60/363,054, filed Mar. 7, 2002, the disclosures of which are incorporated herein by reference in their entireties.

In the first of these methods, the eukaryotic cell to be targeted, or that provides the protein extract having cellular repair enzymes within which a recombinant construct is targeted, is first contacted with an inhibitor of histone deacetylase (HDAC), such as Trichostatin A. In the second of these methods, the sequence-altering oligonucleotide is contacted with the genomic target—either within a cell or within a cell extract—in the presence of lambda beta protein. In the third of these methods, the eukaryotic cell to be targeted, or that provides the protein extract within which a recombinant construct is targeted, is first contacted with hydroxyurea.

Targeting efficiency may also be increased using the methods set forth in U.S. provisional patent application serial No. 60/325,992, filed Sep. 27, 2001; No. 60/337,129, filed Dec. 4, 2001; and No. 60/393,330, filed Jul. 1, 2002, the disclosures of which are incorporated herein by reference in their entireties, and in U.S. provisional application serial No. 60/220,999, filed Jul. 27, 2000; and No. 60/244,989, filed Oct. 30, 2000, the disclosures of which are incorporated herein by reference in their entireties.

In various of these methods, the cell or cell-free extract within which targeting is performed has altered levels or activity of at least one protein from the RAD52 epistasis group, the mismatch repair group or the nucleotide excision repair group, such as reduced levels or activity of at least one protein selected from the group consisting of a homolog, ortholog or paralog of RAD1, RAD51, RAD52, RAD57 and PMS1.

In others of these methods, the cell or cell-free extract within which targeting is performed has increased levels or activity of at least one of RAD10, RAD51, RAD52, RAD54, RAD55, MRE11, PMS1 or XRS2 proteins and decreased levels or activity of at least one other protein selected from the group consisting of RAD1, RAD51, RAD52, RAD57 or PMS1.

The targeting oligonucleotides can introduce more than a single base change in a single step. For example, in an oligonucleotide that is about a 70-mer, with at least one modified residue incorporated on each of the two ends, multiple bases up to 27 nucleotides apart can be targeted. However, when the targeting oligonucleotide includes multiple sequence changes, not all transformants will include all genetic changes: there is a frequency distribution such that the closer the target bases are to each other in the alteration domain, the higher the frequency of change in a given cell. Target bases only two nucleotides apart are changed together in every case that has been analyzed. The farther apart the two target bases are, the less frequent the simultaneous change.

Thus, in creating the coisogenic cell collections of the present invention, targeting oligonucleotides can be used to alter multiple bases at the target locus, rather than just a single base. Furthermore, iterative rounds of targeting can be performed to introduce multiple changes.

In embodiments in which the genome is targeted directly in the cell, the targeting oligonucleotides can be introduced into the cell by any means known in the art, such as through use of poly-cations, cationic lipids, liposomes, polyethylenimine (PEI), electroporation, biolistics, microinjection and other methods known in the art to facilitate cellular uptake; indeed, at times the targeting oligonucleotides can be introduced by simple incubation without any adjunctive means.

In alternative embodiments, the targeting oligonucleotide can be used to introduce the alteration into a genomic DNA construct, with the altered construct thereafter introduced into the cells by known transfection techniques. Typically, the altered construct is far larger than the targeting oligonucleotide, and is sufficient in length to act as a substrate for subsequent homologous recombination with the cellular chromosome.

The coisogenic cell collections of the present invention are useful for screening for the phenotypic effects of changes in the protein sequence encoded at a target locus. Because the cells of the collection are coisogenic, phenotypic differences detected among the cells of the collection can more reliably be ascribed to the differences in sequence at the target locus than in assays using genetically more heterogeneous cells in which additional changes at the target locus, or further changes at loci other than the target locus, can confound the analysis. Furthermore, given the ability readily to include within the collection of the present invention coisogenic cells that collectively have changes at many (including all) of the amino acids encoded at the target locus, the coisogenic cell collections of the present invention are extremely useful for dissecting structure activity relationships within proteins.

Thus, in another aspect, the invention provides a method of identifying genotypes of a target locus that alter a cellular phenotype.

The method comprises assaying each genotypically distinct cell of a coisogenic cell collection of the present invention for a common phenotypic characteristic; the genotypically distinct cells are coisogenic at a desired target locus. From the assay results, at least one genotypically distinct cell is identified within the collection that has an alteration in the assayed phenotypic characteristic (i.e., that exhibits an altered phenotype). Assay results are correlated with the target locus genotype, the correlation identifying genotypes of the target locus that cause an alteration of the cellular phenotype.

The phenotypic characteristic can be any cellular characteristic relevant to the target locus that can be assayed in vitro. A wide variety of such in vitro assays exist, and the principles for design of such assays are by now well known; accordingly, details will not here be presented.

Briefly, however, and solely by way of example, where the target locus is, for example, a steroid receptor, the phenotypic characteristic can be the detectable translocation of the receptor from cytoplasm to nucleus upon contact of the cells to the receptor's cognate ligand, as is described, inter alia, in U.S. Pat. No. 5,989,835. The phenotypic characteristic where the target locus encodes a steroid hormone receptor can alternatively (or additionally) be the expression of a detectable reporter, such as a fluorescent protein (e.g., GFP), driven from a hormone-responsive promoter. In this latter case, the assay depends upon the presence commonly within the cells of the coisogenic collection of a recombinant reporter construct. The recombinant construct can be present within the cells either on an episome or, usefully, integrated into the cellular genome at a locus elsewhere than at the target locus.

Where the target locus encodes a protein known to affect drug responsiveness, such as those described in detail above, the cellular characteristic to be assayed can be as simple and fundamental as degree of cell death, or can alternatively (or additionally) be, for example, the degree of cellular proliferation, degree of metabolic activity, and/or the degree of apoptosis. Appropriate assays are described in several compendia, such as Apoptosis and Cell Proliferation, 2^{nd} ed., Boehringer Mannheim, 1998 (available on-line at http://biochem.boehringer-mannheim.com/prod_inf/manuals/cell_man/acp.pdf), and Poirier (ed.), Apoptosis Techniques and Protocols, Humana Press, 1997 (ISBN: 0896034518), the disclosures of which are incorporated herein by reference. In addition, a wide variety of assay kits are available commercially (e.g., the CellTiter 96® AQueous Non-Radioactive Cell Proliferation Assay, catalogue no. G5421, Promega, Madison, Wis., which is a calorimetric method for determining the number of viable cells in proliferation, cytotoxicity or chemosensitivity assays; the Apoptosis Detection System, Fluorescein, catalogue no. G3250, and the DeadEnd™ Colorimetric Apoptosis Detection System, catalogue no. G7360, both from Promega, Madison, Wis.; ApoAlert™ Apoptosis Detection Kits, Clontech Labs, Palo Alto, Calif., USA).

Where the target locus encodes a protein known to affect drug responsiveness by transport of the drug from the cell interior to the medium, the characteristic to be assayed can alternatively, or additionally, be accumulation or efflux of the drug of interest or proxy therefor. Assays are now well known that permit such accumulation and/or efflux to be measured.

For example, U.S. Pat. Nos. 6,277,655 and 5,872,014, incorporated herein by reference in their entireties, describe assays for activity of ABCB1 (MDR1) based upon fluorescent detection of the degree of cellular accumulation of free calcein after exposure to an acetoxymethyl ester or acetate ester of calcein. Ludescher et al., Br. J. Haematol. 82(1):161-8 (1992) describe a flow cytometric assay for ABCB1 activity based upon degree of intracellular accumulation of rhodamine 123. Gheuens et al., Cytometry 12(7):636-44 (1991), describe flow cytometric double labeling techniques for assay of multidrug resistance. Cano-Gauci et al., Biochem. Biophys. Res. Commun. 167(1):48-53 (1990) describe a fast kinetic analysis assay for drug transport in multidrug resistant cells using a pulsed quench-flow apparatus. Van Acker et al., Leukemia 9:1398-406 (1995) describe a rapid flow cytometric functional assay for P-glycoprotein (encoded by ABCB1) using fluo-3. Other assays are reviewed in Hoffman, “In vitro assays for chemotherapy sensitivity,” Crit. Rev. Oncol. Hematol. 15(2):99-111 (1993); Cree et al., “Tumor chemosensitivity and chemoresistance assays,” Cancer 78(9):2031-2 (1996).

The assay can detect a phenotypic characteristic under static environmental conditions, or can instead can detect a phenotypic characteristic during or after an alteration in the cellular environment. In a useful embodiment of this latter approach, the coisogenic collection of cells is first exposed to a xenobiotic, usefully a known or potential therapeutic agent, and a characteristic of the cells measured thereafter.

Analogously, the assay can detect an equilibrium or otherwise static aspect of the phenotypic characteristic, or can detect kinetic changes in the phenotypic characteristic. For example, in an assay for cytoplasm to nuclear translocation of a steroid receptor, the assay can measure the static nuclear:cytoplasmic ratio of the receptor or can, in the alternative or in addition, measure the rate of translocation from cytoplasm to nucleus.

The assay can be quantitative or qualitative, manual or automated.

From the assay results, at least one cell is identified that has an altered cellular phenotype.

As would be well understood, not all genotypic changes at the target locus will affect the measured phenotypic characteristic. In order, however, to identify residues of the target protein whose change (by way of substitution, deletion, elimination by truncation, etc.) affects a phenotypic characteristic, at least one cell must be identified that has an alteration in the assayed phenotypic characteristic.

That said, data on residues of the protein encoded at the target locus that are tolerant of substitution are also tremendously useful, and in another aspect, therefore, the invention provides the converse method, in which residues tolerant of alteration are identified; in this latter method, correlation of the target locus genotype of cells that do not exhibit change in the assayed phenotypic characteristic identifies residues tolerant of substitution.

As would be readily understood, the “altered phenotype” is altered relative to a chosen control. The control is typically a coisogenic cell, typically in the same collection, that has a desired reference target locus sequence. The desired reference target locus sequence can, for example, be that of the parent cell (typically, cell line) from which the coisogenic cells of the collection have been engineered; that which is most commonly observed in a given population (e.g., the predominant allelic variant of the target locus in a chosen human population); or one chosen based upon prior-determined results of a phenotypic assay.

Following the assay, the results of the phenotypic assay are correlated with the cells' respective target locus genotypes.

The correlation can be performed either before or after identifying, from the assay results, at least one cell with altered cellular phenotype. If performed after the subset with altered phenotypic characteristic is identified, the correlation of phenotype with target locus genotype can be limited to that subset; if performed before the subset with altered phenotype is identified, as would typically be the case in high throughput applications of the methods of the present invention, the correlation of phenotype with target locus genotype would typically be made for all cells of the coisogenic cell collection.

In either case, the correlation of the subset's phenotypic assay results with their respective target locus genotypes identifies those genotypes of the target locus that cause an alteration of the cellular phenotype.

Correlation can be as simple as noting a change in phenotype for a given genotype, such as an increase in cytotoxicity occasioned by contact with a chemotherapeutic agent in a cell having a change in a specific ABCB1 amino acid. Alternatively, or in addition, correlation can be performed using statistical algorithms known in the art.

Where the coisogenic cell collection includes cells that collectively include changes at each amino acid of the protein encoded at the target locus (typically excluding changes of the initiator methionine), correlation of phenotype with genotype can identify all residues of the protein that are critical to its function. Where the coisogenic cell collection includes cells that collectively include each of the 20 natural amino acids at a single residue location, typically a residue previously shown or suspected to contribute to protein function, correlation of phenotype with genotype can identify with precision the structural requirements for function at that residue. Where the coisogenic cell collection includes one or more cells that have a naturally-occurring allelic variant of the target locus, or that encode a protein having a sequence identical to that encoded by a naturally-occurring allelic variant of the target locus, correlation of phenotype with genotype allows the phenotypic effects of such natural variants readily to be assessed in the context of a uniform genetic background.

In one series of embodiments, the method is used to identify genotypes that alter the cellular responsiveness to xenobiotics, which will typically be known or potential therapeutic agents.

In such embodiments, as well as in other embodiments of the methods of the present invention, the target locus at which the cells of the collection are coisogenic can usefully be selected from the group consisting of: CYP1A2, CYP2C17, CYP2D6, CYP2E, CYP3A4, CYP4A11, CYP1B1, CYP1A1, CYP2A6, CYP2A13, CYP2B6, CYP2C8, CYP2C9, CYP11A, CYP2C19, CYP2F1, CYP2J2, CYP3A5, CYP3A7, CYP4B1, CYP4F2, CYP4F3, CYP6D1, CYP6F1, CYP7A1, CYPB, CYP11A, CYP11B1, CYP11B2, CYP17, CYP19, CYP21A2, CYP24, CYP27A1, CYP51, ABCB1, ABCB4, ABCC1, ABCC2, ABCC3, ABCC4, ABCC5, ABCC6, MRP7, ABCC8, ABCC9, ABCC10, ABCC11, ABCC12, EPHX1, EPHX2, LTA4H, TRAG3, GUSB, TMPT, BCRP, HERG, hKCNE2, UDP glucuronosyl transferase (UGT), sulfotransferase, sulfatase, glutathione S-transferase (GST)-alpha, glutathione S-transferase-mu, glutathione S-transferase-pi, ACE, and KCHN2.

The method can usefully include a step, before assay, of contacting the coisogenic cell collection with a xenobiotic, typically a known or potential therapeutic agent. Potential therapeutic agents can be natural products or products of a combinatorial chemical synthesis.

The method can also usefully include a later step, after the correlations have been made, of collecting the correlations into at least one dataset; the dataset is often, but not necessarily, recorded on a computer-readable medium. In such case, the dataset can thereafter usefully be queried, e.g. to predict a cellular phenotype based upon the genotype at the relevant target locus.

Thus, in another aspect, the invention provides a method of predicting a phenotypic characteristic of a cell based upon its genotype at a target locus. The method comprises using the cell's genotype at a chosen target locus, or a unique identifier thereof, as a query to retrieve from a dataset data that report a phenotypic characteristic correlated with the target locus genotype. The dataset that is queried in this method includes correlations from at least five cells that are coisogenic at the target locus. The phenotypic characteristic retrieved from query of the dataset provides a prediction of the cell's phenotypic characteristic.

The target locus “genotype” to be used as a query can be obtained by any means known in the art, including sequencing of the genomic DNA of the target locus, sequencing of the mRNA transcript from the target locus, sequencing of the protein encoded at the target locus, or any of the known methods for identifying allelic variants at a given locus, such as those set forth in U.S. Pat. Nos. 5,952,174, 5,846,710, 5,710,028 and 5,679,524, and those reviewed in Kwok, “High-throughput genotyping assay approaches,” Pharmacogenomics 1 (1):95-100 (2000), the disclosures of which are incorporated herein by reference. In addition, apparatus is now available commercially that permits the ready identification of allelic variants at a chosen target locus, such as the SniPer™ High Throughput SNP Scoring System (Amersham Pharmacia Biotech, Piscataway, N.J., USA) and the SNPstream™ (Orchid Biosciences, Princeton, N.J., USA).

The cell for which the genotype is to be used as query can be a cultured cell or, alternatively, can be a noncultured cell derived directly from a eukaryotic organism. In the latter case, the genotype can be obtained, for example, from cells, such as circulating blood cells, that are replenishable in vivo. The cell for which the genotype is determined can be normally present in the eukaryotic organism or can be aberrant or otherwise diseased.

Usefully, the target locus genotype can be obtained from cells of a human being.

The query itself can include the entirety of the nucleic acid or protein sequence of the target locus, a portion of the nucleic acid or protein sequence of the target locus, even a single nucleotide or protein identifier and base or residue number that can serve as a unique identifier of the target locus genotype. Methods are well known in the bioinformatic arts for querying databases having sequence-related information.

The dataset to be queried includes correlations derived from at least five cells that are coisogenic at the target locus. Typically, the coisogenic cells will have been a cell collection according to the present invention.

Where the cellular genotype used as query is derived from a human being, the above-described methods provide a streamlined approach to pharmacogenomic analysis.

An antecedent to traditional pharmacogenomic studies is the identification of a large number of naturally-occurring allelic variants, and correlation of the naturally-occurring alleles with naturally-occurring clinical phenotypes. Only then can a patient's genotype be used to predict the patient's probably clinical phenotype.

In contrast, the coisogenic collections of eukaryotic cells of the present invention allow all possible alleles readily to be constructed, and the resulting cellular phenotypes to be correlated with target locus genotype. Where the cellular phenotype can correlated with the phenotype of the entire organism, as can readily be done with loci that affect responsiveness to xenobiotics, the dataset of correlated phenotypes can provide reliable phenotypic predictions, even for alleles that had not previously been identified within the natural population.

Thus, in certain particularly useful embodiments, the query genotype is from a human cell, and the target locus is selected from the group consisting of CYP1A2, CYP2C17, CYP2D6, CYP2E, CYP3A4, CYP4A11, CYP1B1, CYP1A1, CYP2A6, CYP2A13, CYP2B6, CYP2C8, CYP2C9, CYP11A, CYP2C19, CYP2F1, CYP2J2, CYP3A5, CYP3A7, CYP4B1, CYP4F2, CYP4F3, CYP6D1, CYP6F1, CYP7A1, CYP8, CYP11A, CYP11B1, CYP11B2, CYP17, CYP19, CYP21A2, CYP24, CYP27A1, CYP51, ABCB1, ABCB4, ABCC1, ABCC2, ABCC3, ABCC4, ABCC5, ABCC6, MRP7, ABCC8, ABCC9, ABCC10, ABCC11, ABCC12, EPHX1, EPHX2, LTA4H, TRAG3, GUSB, TMPT, BCRP, HERG, hKCNE2, UDP glucuronosyl transferase (UGT), sulfotransferase, sulfatase, glutathione S-transferase (GST)-alpha, glutathione S-transferase-mu, glutathione S-transferase-pi, ACE, and KCHN2, and the cellular phenotypic characteristic can usefully be cellular responsiveness to a xenobiotic; in such case, the prediction can be a prediction of an individual's potential responsiveness to that xenobiotic agent.

The following examples are offered for purpose of illustration and not by way of limitation.

### EXAMPLE 1

### Coisogenic Eukaryotic Cell Collections Having Natural Allelic Variants of ABCB1 (MDR1)

Targeting oligos are used to create a cell collection coisogenic at the human ABCB1 (MDR1) locus.

The targeting oligonucleotides include terminal modifications as set forth above, including at least one phosphorothiate linkage, and are introduced in parallel into separate aliquots of HBL100 cells using standard techniques. Potential cellular tranformants are propagated in vitro, cloned, and clonal cell lines having the desired targeted change identified by sequencing DNA amplified from the ABCB1 locus.

The targeting oligos have sequences (presented in Table 35, below) designed to create natural allelic variants of the ABCB1 gene, creating a legacy-free, perfectly coisogenic cell collection in which the naturally occurring alleles of ABCB1 are presented on the identical genetic background of a human breast epithelial cell line.

The left-most column of the table identifies the alteration that converts the wild type to the variant allele, at both the amino acid and the nucleotide level. At the amino acid level, mutations are presented according to the following standard nomenclature. The centered number identifies the position of the mutated codon in the protein sequence; to the left of the number is the wild type residue and to the right of the number is the mutant codon. At the nucleic acid level, the entire triplet of the wild type and mutated codons is shown.

The middle column presents, for each alteration (mutation), four oligonucleotides capable of changing the wild type sequence site-specifically to the identified allelic variant.

All oligonucleotides are presented, per convention, in the 5′ to 3′ orientation. The nucleotide that effects the change in the genome is underlined and presented in bold.

The first of the four oligonucleotides for each mutation is a 121 nt oligonucleotide centered about the altering (“repair”) nucleotide. The second oligonucleotide, its reverse complement, targets the opposite strand of the DNA duplex for change (“repair”). The third oligonucleotide is the minimal 17 nt domain of the first oligonucleotide, also centered about the repair nucleotide. The fourth oligonucleotide is the reverse complement of the third, and thus represents the minimal 17 nt domain of the second.

The third column of the table presents the SEQ ID NO: of the respective targeting oligonucleotide.

TABLE 35

ABCB1 (MDR1) Targeting Oligos to Create Natural
Alleles

| | |
|---|---|
| Allelic Variation | Sequence of Targeting Oligos SEQ ID NO: |

| | | |
|---|---|---|
| Asn21Asp | ATGGATCTTGAAGGGGA | 1 |
| AAT-GAT | | |
| | CCGCAATGGAGGAGCAA | |
| | AGAAGAAGAACTTTTTTA | |
| | AACTGAACGATAAAAGG | |
| | TAACTAGCTTGTTTCATT | |
| | TTCATAGTTTACATAGTT | |
| | GCGAGATTTGAGTAAT | |
| | ATTACTCAAATCTCGCAA | 2 |
| | CTATGTAAACTATGAAAA | |
| | TGAAACAAGCTAGTTACC | |
| | TTTTATCGTTCAGTTTAA | |
| | AAAAGTTCTTCTTCTTTG | |
| | CTCCTCCATTGCGGTCC | |
| | CCTTCAAGATCCAT | |
| | AACTGAACGATAAAAGG | 3 |
| | CCTTTTATCGTTCAGTT | 4 |
| Phe103Ser | AAGAGACATAAATGGTAT | 5 |
| TTC-TCC | | |
| | GTTTGTTTTGTGGTGGTC | |
| | TAGGTGATATCAATGATA | |
| | CAGGGTCCTTCATGAAT | |
| | CTGGAGGAAGACATGAC | |
| | CAGGTAATTAGACATTCT | |
| | CCTTACTATTGTTAA | |
| | TTAACAATAGTAAGGAGA | 6 |
| | ATGTCTAATTACCTGGTC | |
| | ATGTCTTCCTCCAGATTC | |
| | ATGAAGGACCCTGTATC | |
| | ATTGATATCACCTAGACC | |
| | ACCACAAAACAAACATAC | |
| | CATTTATGTCTCTT | |
| | TACAGGGTCCTTCATGA | 7 |
| | TCATGAAGGACCCTGTA | 8 |
| Phe103Leu | AAAGAGACATAAATGGTA | 9 |
| TTC-CTC | | |
| | TGTTTGTTTTGTGGTGGT | |
| | CTAGGTGATATCAATGAT | |
| | ACAGGGCTCTTCATGAA | |
| | TCTGGAGGAAGACATGA | |
| | CCAGGTAATTAGACATTC | |
| | TCCTTACTATTGTTA | |
| | TAACAATAGTAAGGAGAA | 10 |
| | TGTCTAATTACCTGGTCA | |
| | TGTCTTCCTCCAGATTCA | |
| | TGAAGAGCCCTGTATCA | |
| | TTGATATCACCTAGACCA | |
| | CCACAAAACAAACATACC | |
| | ATTTATGTCTCTTT | |
| | ATACAGGGCTCTTCATG | 11 |
| | CATGAAGAGCCCTGTAT | 12 |
| Gly185Val | TTCTGACAATTATTTCTA | 13 |
| GGA-GTA | | |
| | ACACTATCTGTTCTTTCA | |
| | GTGATGTCTCCAAGATTA | |
| | ATGAAGTAATTGGTGACA | |
| | AAATTGGAATGTTCTTTC | |
| | AGTCAATGGCAACATTTT | |
| | TCACTGGGTTTAT | |
| | ATAAACCCAGTGAAAAAT | 14 |
| | GTTGCCATTGACTGAAA | |
| | GAACATTCCAATTTTGTC | |
| | ACCAATTACTTCATTAAT | |
| | CTTGGAGACATCACTGA | |
| | AAGAACAGATAGTGTTA | |
| | GAAATAATTGTCAGAA | |
| | TAATGAAGTAATTGGTG | 15 |
| | CACCAATTACTTCATTA | 16 |
| Ser400Asn | AGAGTGGGCACAAACCA | 17 |
| AGT-AAT | | |
| | GATAATATTAAGGGAAAT | |
| | TTGGAATTCAGAAATGTT | |
| | CACTTCAATTACCCATCT | |
| | CGAAAAGAAGTTAAGGT | |
| | ACAGTGATAAATGATTAA | |
| | TCAACAATTAATCTA | |
| | TAGATTAATTGTTGATTA | 18 |
| | ATCATTTATCACTGTACC | |
| | TTAACTTCTTTTCGAGAT | |
| | GGGTAATTGAAGTGAAC | |
| | ATTTCTGAATTCCAAATT | |
| | TCCCTTAATATTATCTGG | |
| | TTTGTGCCCACTCT | |
| | TCACTTCAATTACCCAT | 19 |
| | ATGGGTAATTGAAGTGA | 20 |
| Val801Met | GGAGCTGAGAGTCTCAT | 21 |
| GTG-ATG | | |
| | AAACAGCTTTAAGGTAAT | |
| | AAAATCATTTTCTGTGCC | |
| | ACAGGATATGAGTTGGT | |
| | TTGATGACCCTAAAAACA | |
| | CCACTGGAGCATTGACT | |
| | ACCAGGCTCGCCAATG | |
| | CATTGGCGAGCCTGGTA | 22 |
| | GTCAATGCTCCAGTGGT | |
| | GTTTTTAGGGTCATCAAA | |
| | CCAACTCATATCCTGTG | |
| | GCACAGAAAATGATTTTA | |
| | TTACCTTAAAGCTGTTTA | |
| | TGAGACTCTCAGCTCC | |
| | CACAGGATATGAGTTGG | 23 |
| | CCAACTCATATCCTGTG | 24 |
| Ile829Val | AGCATGAGTTGTGAAGA | 25 |
| ATA-GTA | | |
| | TAATATTTTTAAAATTTCT | |
| | CTAATTTGTTTTGTTTTG | |
| | CAGGCTGTAGGTTCCAG | |
| | GCTTGCTGTAATTACCCA | |
| | GAATATAGCAAATCTTGG | |
| | GACAGGAATAATTA | |
| | TAATTATTCCTGTCCCAA | 26 |
| | GATTTGCTATATTCTGGG | |
| | TAATTACAGCAAGCCTG | |
| | GAACCTACAGCCTGCAA | |
| | AACAAAACAAATTAGAGA | |
| | AATTTTAAAAATATTATCT | |
| | TCACAACTCATGCT | |
| | TGCAGGCTGTAGGTTCC | 27 |
| | GGAACCTACAGCCTGCA | 28 |
| Ser893Ala | GTTGTTGAAATGAAAATG | 29 |
| TCT-GCT | | |
| | TTGTCTGGACAAGGACT | |
| | GAAAGATAAGAAAGAAC | |
| | TAGAAGGTGCTGGGAAG | |
| | GTGAGTCAAACTAAATAT | |
| | GATTGATTAATTAAGTAG | |
| | AGTAAAGTATTCTAAT | |
| | ATTAGAATACTTTACTCT | 30 |
| | ACTTAATTAATCAATCAT | |
| | ATTTAGTTTGACTCACCT | |
| | TCCCAGCACCTTCTAGTT | |
| | CTTTCTTATCTTTCAGTG | |
| | CTTGTCCAGACAACATTT | |
| | TCATTTCAACAAC | |
| | TAGAAGGTGCTGGGAAG | 31 |
| | CTTCCCAGCACCTTCTA | 32 |
| Ser893Thr | GTTGTTGAAATGAAAATG | 33 |
| TCT-ACT | | |
| | TTGTCTGGACAAGCACT | |
| | GAAAGATAAGAAAGAAC | |
| | TAGAAGGTACTGGGAAG | |
| | GTGAGTCAAACTAAATAT | |
| | GATTGATTAATTAAGTAG | |
| | AGTAAAGTATTCTAAT | |
| | ATTAGAATACTTTACTCT | 34 |
| | ACTTAATTAATCAATCAT | |
| | ATTTAGTTTGACTCACCT | |
| | TCCCAGTACCTTCTAGTT | |
| | CTTTCTTATCTTTCAGTG | |
| | CTTGTCCAGACAACATTT | |
| | TCATTTCAACAAC | |
| | TAGAAGGTACTGGGAAG | 35 |
| | CTTCCCAGTACCTTCTA | 36 |
| Ala999Thr | TCAGCTGTTGTCTTTGGT | 37 |
| GCC-ACC | | |
| | GCCATGGCCGTGGGGC | |
| | AAGTCAGTTCATTTGCTC | |
| | CTGACTATACCAAAGCC | |
| | AAAATATCAGCAGCCCA | |
| | CATCATCATGATCATTGA | |
| | AAAAACCCCTTTGATTG | |
| | CAATCAAAGGGGTTTTTT | 38 |
| | CAATGATCATGATGATGT | |
| | GGGCTGCTGATATTTTG | |
| | GCTTTGGTATAGTCAGG | |
| | AGCAAATGAACTGACTT | |
| | GCCCCACGGCCATGGCA | |
| | CCAAAGACAACAGCTGA | |
| | CTGACTATACCAAAGCC | 39 |
| | GGCTTTGGTATAGTCAG | 40 |
| Gln1107Pro | GATCTGTGAACTCTTGTT | 41 |
| CAG-CCG | | |
| | TTCAGCTGCTTGATGGC | |
| | AAAGAAATAAAGCGACT | |
| | GAATGTTCCGTGGCTCC | |
| | GAGCACACCTGGGCATC | |
| | GTGTCCCAGGAGCCCAT | |
| | CCTGTTTGACTGCAGCA | |
| | T | |
| | ATGCTGCAGTCAAACAG | 42 |
| | GATGGGCTCCTGGGACA | |
| | CGATGCCCAGGTGTGCT | |
| | CGGAGCCACGGAACATT | |
| | CAGTCGCTTTATTTCTTT | |
| | GCCATCAAGCAGCTGAA | |
| | AACAAGAGTTCACAGAT | |
| | C | |
| | GAATGTTCCGTGGCTCC | 43 |
| | GGAGCCACGGAACATTC | 44 |

Aliquots of the coisogenic cell collection are thereafter separately contacted with a variety of chemotherapeutic agents presently used for, or contemplated for use in, treatment of breast adenocarcinoma, and alleles that increase or decrease sensitivity to the cytotoxic effects of the agents are identified.

### EXAMPLE 2

### Coisogenic Eukaryotic Cell Collections Having Natural Allelic Variants of CYP2D6

Targeting oligos are used to create a cell collection coisogenic at the human CYP2D6 locus.

The targeting oligonucleotides include terminal modifications as set forth above, including at least one phosphorothiate linkage, and are introduced in parallel into separate aliquots of HBL100 cells using standard techniques. Potential cellular tranformants are propagated in vitro, cloned, and clonal cell lines having the desired targeted change identified by sequencing DNA amplified from the CYP2D6 locus.

The targeting oligos have sequences (presented in Table 36, below) designed to create natural allelic variants of the CYP2D6 gene, creating a legacy-free, perfectly coisogenic cell collection in which the naturally occurring alleles of CYP2D6 are presented on the identical genetic background of a human breast epithelial cell line.

The left-most column of the table identifies the alteration that converts the wild type to the variant allele, at both the amino acid and the nucleotide level. At the amino acid level, mutations are presented according to the following standard nomenclature. The centered number identifies the position of the mutated codon in the protein sequence; to the left of the number is the wild type residue and to the right of the number is the mutant codon. At the nucleic acid level, the entire triplet of the wild type and mutated codons is shown.

The middle column presents, for each alteration (mutation), four oligonucleotides capable of changing the wild type sequence site-specifically to the identified allelic variant.

All oligonucleotides are presented, per convention, in the 5′ to 3′ orientation. The nucleotide that effects the change in the genome is underlined and presented in bold.

The first of the four oligonucleotides for each mutation is a 121 nt oligonucleotide centered about the altering (“repair”) nucleotide. The second oligonucleotide, its reverse complement, targets the opposite strand of the DNA duplex for change (“repair”). The third oligonucleotide is the minimal 17 nt domain of the first oligonucleotide, also centered about the repair nucleotide. The fourth oligonucleotide is the reverse complement of the third, and thus represents the minimal 17 nt domain of the second.

The third column of the table presents the SEQ ID NO: of the respective targeting oligonucleotide.

TABLE 36

CYP2D6 Targeting Oligos to
Create Natural Alleles

| | | |
|---|---|---|
| Allelic | | SEQ ID |
| Variation | Sequence of Targeting Oligos | NO: |

| | | |
|---|---|---|
| Val7Met | GCCAGGTGTGTCCAGAGGAGCCCATTTGGTAGT | 45 |
| GTG-ATG | | |
| | GAGGCAGGTATGGGGCTAGAAGCACTGATGCCC | |
| | CTGGCCGTGATAGTGGCCATCTTCCTGCTCCTGG | |
| | TGGACCTGATGCACCGGCGCC | |
| | GGCGCCGGTGCATCAGGTCCACCAGGAGCAGGA | 46 |
| | AGATGGCCACTATCACGGCCAGGGGCATCAGTG | |
| | CTTCTAGCCCCATACCTGCCTCACTACCAAATGG | |
| | GCTCCTCTGGACACACCTGGC | |
| | AAGCACTGATGCCCCTG | 47 |
| | CAGGGGCATCAGTGCTT | 48 |
| Val11Met | CAGAGGAGCCCATTTGGTAGTGAGGCAGGTATG | 49 |
| GTG-ATG | | |
| | GGGCTAGAAGCACTGGTGCCCCTGGCCATGATA | |
| | GTGGCCATCTTCCTGCTCCTGGTGGACCTGATGC | |
| | ACCGGCGCCAACGCTGGGCTG | |
| | CAGCCCAGCGTTGGCGCCGGTGCATCAGGTCCA | 50 |
| | CCAGGAGCAGGAAGATGGCCACTATCATGGCCA | |
| | GGGGCACCAGTGCTTCTAGCCCCATACCTGCCTC | |
| | ACTACCAAATGGGCTCCTCTG | |
| | CCCTGGCCATGATAGTG | 51 |
| | CACTATCATGGCCAGGG | 52 |
| Arg26His | TGGTGCCCCTGGCCGTGATAGTGGCCATCTTCCT | 53 |
| CGC-CAC | | |
| | GCTCCTGGTGGACCTGATGCACCGGCACCAACG | |
| | CTGGGCTGCACGCTACCCACCAGGCCCCCTGCC | |
| | ACTGCCCGGGCTGGGCAACCT | |
| | AGGTTGCCCAGCCCGGGCAGTGGCAGGGGGCC | 54 |
| | TGGTGGGTAGCGTGCAGCCCAGCGTTGGTGCCG | |
| | GTGCATCAGGTCCACCAGGAGCAGGAAGATGGC | |
| | CACTATCACGGCCAGGGGCACCA | |
| | GCACCGGCACCAACGCT | 55 |
| | AGCGTTGGTGCCGGTGC | 56 |
| Arg28Cys | CCCCTGGCCGTGATAGTGGCCATCTTCCTGCTCC | 57 |
| CGC-TGC | | |
| | TGGTGGACCTGATGCACCGGCGCCAATGCTGGG | |
| | CTGCACGCTACCCACCAGGCCCCCTGCCACTGC | |
| | CCGGGCTGGGCAACCTGCTGC | |
| | GCAGCAGGTTGCCCAGCCCGGGCAGTGGCAGG | 58 |
| | GGGCCTGGTGGGTAGCGTGCAGCCCAGCATTGG | |
| | CGCCGGTGCATCAGGTCCACCAGGAGCAGGAAG | |
| | ATGGCCACTATCACGGCCAGGGG | |
| | GGCGCCAATGCTGGGCT | 59 |
| | AGCCCAGCATTGGCGCC | 60 |
| Pro34Ser | GCCATCTTCCTGCTCCTGGTGGACCTGATGCACC | 61 |
| CCA-TCA | | |
| | GGCGCCAACGCTGGGCTGCACGCTACTCACCAG | |
| | GCCCCCTGCCACTGCCCGGGCTGGGCAACCTGC | |
| | TGCATGTGGACTTCCAGAACA | |
| | TGTTCTGGAAGTCCACATGCAGCAGGTTGCCCAG | 62 |
| | CCCGGGCAGTGGCAGGGGGCCTGGTGAGTAGC | |
| | GTGCAGCCCAGCGTTGGCGCCGGTGCATCAGGT | |
| | CCACCAGGAGCAGGAAGATGGC | |
| | CACGCTACTCACCAGGC | 63 |
| | GCCTGGTGAGTAGCGTG | 64 |
| Gly42Arg | CTGATGCACCGGCGCCAACGCTGGGCTGCACGC | 65 |
| GGG-AGG | | |
| | TACCCACCAGGCCCCCTGCCACTGCCCAAGGCTG | |
| | GGCAACCTGCTGCATGTGGACTTCCAGAACACAC | |
| | CATACTGCTTCGACCAGGTGA | |
| | TCACCTGGTCGAAGCAGTATGGTGTGTTCTGGAA | 66 |
| | GTCCACATGCAGCAGGTTGCCCAGCCTGGGCAG | |
| | TGGCAGGGGGCCTGGTGGGTAGCGTGCAGCCCA | |
| | GCGTTGGCGCCGGTGCATCAG | |
| | CACTGCCCAGGCTGGGC | 67 |
| | GCCCAGCCTGGGCAGTG | 68 |
| Ala85Val | TCGGGGACGTGTTCAGCCTGCAGCTGGCCTGGA | 69 |
| GCG-GTG | | |
| | CGCCGGTGGTCGTGCTCAATGGGCTGGTGGCCG | |
| | TGCGCGAGGCGCTGGTGACCCACGGCGAGGACA | |
| | CCGCCGACCGCCCGCCTGTGCC | |
| | GGCACAGGCGGGCGGTCGGCGGTGTCCTCGCC | 70 |
| | GTGGGTCACCAGCGCCTCGCGCACGGCCACCAG | |
| | CCCATTGAGCACGACCACCGGCGTCCAGGCCAG | |
| | CTGCAGGCTGAACACGTCCCCGA | |
| | TGGGCTGGTGGCCGTGC | 71 |
| | GCACGGCCAACCAGGCCCA | 72 |
| Leu91Met | CTGCAGCTGGCCTGGACGCCGGTGGTCGTGCTC | 73 |
| CTG-ATG | | |
| | AATGGGCTGGCGGCCGTGCGCGAGGCGATGGT | |
| | GACCCACGGCGAGGACACCGCCGACCGCCCGC | |
| | CTGTGCCCATCACCCAGATCCTGG | |
| | CCAGGATCTGGGTGATGGGCACAGGCGGGCGGT | 74 |
| | CGGCGGTGTCCTCGCCGTGGGTCACCATCGCCT | |
| | CGCGCACGGCCGCCAGCCCATTGAGCACGACCA | |
| | CCGGCGTCCAGGCCAGCTGCAG | |
| | GCGAGGCGATGGTGACC | 75 |
| | GGTCACCATCGCCTCGC | 76 |
| His94Arg | CCTGGACGCCGGTGGTCGTGCTCAATGGGCTGG | 77 |
| CAC-CGC | | |
| | CGGCCGTGCGCGAGGCGCTGGTGACCCGCGGC | |
| | GAGGACACCGCCGACCGCCCGCCTGTGCCCATC | |
| | ACCCAGATCCTGGGTTTCGGGCC | |
| | GGCCCGAAACCCAGGATCTGGGTGATGGGCACA | 78 |
| | GGCGGGCGGTCGGCGGTGTCCTCGCCGCGGGT | |
| | CACCAGCGCCTCGCGCACGGCCGCCAGCCCATT | |
| | GAGCACGACCACCGGCGTCCAGG | |
| | GGTGACCCGCGGCGAGG | 79 |
| | CCTCGCCGCGGGTCACC | 80 |
| Thr107Ile | TGCGCGAGGCGCTGGTGACCCACGGCGAGGACA | 81 |
| ACC-ATC | | |
| | CCGCCGACCGCCCGCCTGTGCCCATCATCCAGA | |
| | TCCTGGGTTTCGGGCCGCGTTCCCAAGGCAAGC | |
| | AGCGGTGGGGACAGAGACAGAT | |
| | ATCTGTCTCTGTCCCCACCGCTGCTTGCCTTGGG | 82 |
| | AACGCGGCCCGAAACCCAGGATCTGGATGATGG | |
| | GCACAGGCGGGCGGTCGGCGGTGTCCTCGCCG | |
| | TGGGTCACCAGCGCCTCGCGCA | |
| | GCCCATCATCCAGATCC | 83 |
| | GGATCTGGATGATGGGC | 84 |
| Val136Met | CCCCCAGGGGTGTTCCTGGCGCGCTATGGGCCC | 85 |
| GTG-ATG | | |
| | GCGTGGCGCGAGCAGAGGCGCTTCTCCATGTCC | |
| | ACCTTGCGCAACTTGGGCCTGGGCAAGAAGTCG | |
| | CTGGAGCAGTGGGTGACCGAGG | |
| | CCTCGGTCACCCACTGCTCCAGCGACTTCTTGCC | 86 |
| | CAGGCCCAAGTTGCGCAAGGTGGACATGGAGAA | |
| | GCGCCTCTGCTCGCGCCACGCGGGCCCATAGCG | |
| | CGCCAGGAACACCCCTGGGGG | |
| | GCTTCTCCATGTCCACC | 87 |
| | GGTGGACATGGAGAAGC | 88 |
| Gln151Glu | CAGAGGCGCTTCTCCGTGTCCACCTTGCGCAACT | 89 |
| CAG-GAG | | |
| | TGGGCCTGGGCAAGAAGTCGCTGGAGGAGTGGG | |
| | TGACCGAGGAGGCCGCCTGCCTTTGTGCCGCCT | |
| | TCGCCAACCACTCCGGTGGGT | |
| | ACCCACCGGAGTGGTTGGCGAAGGCGGCACAAA | 90 |
| | GGCAGGCGGCCTCCTCGGTCACCCACTCCTCCA | |
| | GCGACTTCTTGCCCAGGCCCAAGTTGCGCAAGGT | |
| | GGACACGGAGAAGCGCCTCTG | |
| | CGCTGGAGGAGTGGGTG | 91 |
| | CACCCACTCCTCCAGCG | 92 |
| Asn166Asp | AAGAAGTCGCTGGAGCAGTGGGTGACCGAGGAG | 93 |
| AAC-GAC | | |
| | GCCGCCTGCCTTTGTGCCGCCTTCGCCGACCACT | |
| | CCGGTGGGTGATGGGCAGAAGGGCACAAAGCGG | |
| | GAACTGGGAAGGCGGGGGACG | |
| | CGTCCCCCGCCTTCCCAGTTCCCGCTTTGTGCCC | 94 |
| | TTCTGCCCATCACCCACCGGAGTGGTCGGCGAA | |
| | GGCGGCACAAAGGCAGGCGGCCTCCTCGGTCAC | |
| | CCACTGCTCCAGCGACTTCTT | |
| | CCTTCGCCGACCACTCC | |
| | CCACTGCTCCAGCGACTTCTT | |
| | CCTTCGCCGACCACTCC | 95 |
| | GGAGTGGTCGGCGAAGG | 96 |
| Gly169Arg | CTGGAGCAGTGGGTGACCGAGGAGGCCGCCTGC | 97 |
| GGA-AGA | | |
| | CTTTGTGCCGCCTTCGCCAACCACTCCAGTGGGT | |
| | GATGGGCAGAAGGGCACAAAGCGGGAACTGGGA | |
| | AGGCGGGGGACGGGGAAGGCG | |
| | CGCCTTCCCCGTCCCCCGCCTTCCCAGTTCCCGC | 98 |
| | TTTGTGCCCTTCTGCCCATCACCCACTGGAGTGG | |
| | TTGGCGAAGGCGGCACAAAGGCAGGCGGCCTCC | |
| | TCGGTCACCCACTGCTCCAG | |
| | ACCACTCCAGTGGGTGA | 99 |
| | TCACCCACTGGAGTGGT | 100 |
| Arg173Cys | AGGCGGGGGACGGGGAAGGCGACCCCTTACCC | 101 |
| CGC-TGC | | |
| | GCATCTCCCACCCCCAGGACGCCCCTTTTGCCCC | |
| | AACGGTCTCTTGGACAAAGCCGTGAGCAACGTGA | |
| | TCGCCTCCCTCACCTGCGGGC | |
| | GCCCGCAGGTGAGGGAGGCGATCACGTTGCTCA | 102 |
| | CGGCTTTGTCCAAGAGACCGTTGGGGCAAAAGG | |
| | GGCGTCCTGGGGGTGGGAGATGCGGGTAAGGG | |
| | GTCGCCTTCCCCGTCCCCCGCCT | |
| | GCCCCTTTTGCCCCAAC | 103 |
| | GTTGGGGCAAAAGGGGC | 104 |
| Arg201His | CCGTGAGCAACGTGATCGCCTCCCTCACCTGCG | 105 |
| CGC-CAC | | |
| | GGCGCCGCTTCGAGTACGACGACCCTCACTTCCT | |
| | CAGGCTGCTGGACCTAGCTCAGGAGGGACTGAA | |
| | GGAGGAGTCGGGCTTTCTGCG | |
| | CGCAGAAAGCCCGACTCCTCCTTCAGTCCCTCCT | 106 |
| | GAGCTAGGTCCAGCAGCCTGAGGAAGTGAGGGT | |
| | CGTCGTACTCGAAGCGGCGCCCGCAGGTGAGGG | |
| | AGGCGATCACGTTGCTCACGG | |
| | CGACCCTCACTTCCTCA | 107 |
| | TGAGGAAGTGAGGGTCG | 108 |
| Gly212Glu | GGCGCCGCTTCGAGTACGACGACCCTCGCTTCC | 109 |
| GGA-GAA | | |
| | TCAGGCTGCTGGACCTAGCTCAGGAGGAACTGA | |
| | AGGAGGAGTCGGGCTTTCTGCGCGAGGTGCGGA | |
| | GCGAGAGACCGAGGAGTCTCTG | |
| | CAGAGACTCCTCGGTCTCTCGCTCCGCACCTCGC | 110 |
| | GCAGAAAGCCCGACTCCTCCTTCAGTTCCTCCTG | |
| | AGCTAGGTCCAGCAGCCTGAGGAAGCGAGGGTC | |
| | GTCGTACTCGAAGCGGCGCC | |
| | TCAGGAGGAACTGAAGG | 111 |
| | CCTTCAGETCCTCCTGA | 112 |
| Leu231Pro | CAGGAGGGATTGAGACCCCGTTCTGTCTGGTGTA | 113 |
| CTG-CCG | | |
| | GGTGCTGAATGCTGTCCCCGTCCTCCCGCATATC | |
| | CCAGCGCTGGCTGGCAAGGTCCTACGCTTCCAAA | |
| | AGGCTTTCCTGACCCAGCT | |
| | AGCTGGGTCAGGAAAGCCTTTTGGAAGCGTAGG | 114 |
| | ACCTTGCCAGCCAGCGCTGGGATATGCGGGAGG | |
| | ACGGGGACAGCATTCAGCACCTACACCAGACAGA | |
| | ACGGGGTCTCAATCCCTCCTG | |
| | CGTCCTCCCGCATATCC | 115 |
| | GGATATGCGGGAGGACG | 116 |
| Ala237Ser | CCGTTCTGTCTGGTGTAGGTGCTGAATGCTGTCC | 117 |
| GCT-TCT | | |
| | CCGTCCTCCTGCATATCCCAGCGCTGTCTGGCAA | |
| | GGTCCTACGCTTCCAAAAGGCTTTCCTGACCCAG | |
| | CTGGATGAGCTGCTAACTG | |
| | CAGTTAGCAGCTCATCCAGCTGGGTCAGGAAAGC | 118 |
| | CTTTTGGAAGCGTAGGACCTTGCCAGACAGCGCT | |
| | GGGATATGCAGGAGGACGGGGACAGCATTCAGC | |
| | ACCTACACCAGACAGAACGG | |
| | CAGCGCTGTCTGGCAAG | 119 |
| | CTTGCCAGACAGCGCTG | 120 |
| Arg296Cys | GCTCTCGGCCCTGCTCAGGCCAAGGGGAACCCT | 121 |
| CGC-TGC | | |
| | GAGAGCAGCTTCAATGATGAGAACCTGTGCATAG | |
| | TGGTGGCTGACCTGTTCTCTGCCGGGATGGTGA | |
| | CCACCTCGACCACGCTGGCCT | |
| | AGGCCAGCGTGGTCGAGGTGGTCACCATCCCGG | 122 |
| | CAGAGAACAGGTCAGCCACCACTATGCACAGGTT | |
| | CTCATCATTGAAGCTGCTCTCAGGGTTCCCCTTG | |
| | GCCTGAGCAGGGCCGAGAGC | |
| | AGAACCTGTGCATAGTG | 123 |
| | CACTATGCACAGGTTCT | 124 |
| Ile297Leu | CTCGGCCCTGCTCAGGCCAAGGGGAACCCTGAG | 125 |
| ATA-CTA | | |
| | AGCAGCTTCAATGATGAGAACCTGCGCCTAGTGG | |
| | TGGCTGACCTGTTCTCTGCCGGGATGGTGACCAC | |
| | CTCGACCACGCTGGCCTGGG | |
| | CCCAGGCCAGCGTGGTCGAGGTGGTCACCATCC | 126 |
| | CGGCAGAGAACAGGTCAGCCACCACTAGGCGCA | |
| | GGTTCTCATCATTGAAGCTGCTCTCAGGGTTCCC | |
| | CTTGGCCTGAGCAGGGCCGAG | |
| | ACCTGCGCCTAGTGGTG | 127 |
| | CACCACTAGGCGCAGGT | 128 |
| Ala300Gly | CTCAGGCCAAGGGGAACCCTGAGAGCAGCTTCA | 129 |
| GCG-GGT | | |
| | ATGATGAGAACCTGCGCATAGTGGTGGGTGACCT | |
| | GTTCTCTGCCGGGATGGTGACCACCTCGACCAC | |
| | GCTGGCCTGGGGCCTCCTGCT | |
| | AGCAGGAGGCCCCAGGCCAGCGTGGTCGAGGT | 130 |
| | GGTCACCATCCCGGCAGAGAACAGGTCACCCAC | |
| | CACTATGCGCAGGTTCTCATCATTGAAGCTGCTC | |
| | TCAGGGTTCCCCTTGGCCTGAG | |
| | AGTGGTGGGTGACCTGT | 131 |
| | ACAGGTCACCCACCACT | 132 |
| Asp301Asn | CAGGCCAAGGGGAACCCTGAGAGCAGCTTCAAT | 133 |
| GAC-AAC | | |
| | GATGAGAACCTGCGCATAGTGGTGGCTAACCTGT | |
| | TCTCTGCCGGGATGGTGACCACCTCGACCACGCT | |
| | GGCCTGGGGCCTCCTGCTCA | |
| | TGAGCAGGAGGCCCCAGGCCAGCGTGGTCGAG | 134 |
| | GTGGTCACCATCCCGGCAGAGAACAGGTTAGCC | |
| | ACCACTATGCGCAGGTTCTCATCATTGAAGCTGC | |
| | TCTCAGGGTTCCCCTTGGCCTG | |
| | TGGTGGCTAACCTGTTC | 135 |
| | GAACAGGTTAGCCACCA | 136 |
| Ser311Leu | ATGATGAGAACCTGCGCATAGTGGTGGCTGACCT | 137 |
| TCG-TTG | | |
| | GTTCTCTGCCGGGATGGTGACCACCTTGACCACG | |
| | CTGGCCTGGGGCCTCCTGCTCATGATCCTACATC | |
| | CGGATGTGCAGCGTGAGCC | |
| | GGCTCACGCTGCACATCCGGATGTAGGATCATGA | 138 |
| | GCAGGAGGCCCCAGGCCAGCGTGGTCAAGGTG | |
| | GTCACCATCCCGGCAGAGAACAGGTCAGCCACC | |
| | ACTATGCGCAGGTTCTCATCAT | |
| | GACCACCTTGACCACGC | 139 |
| | GCGTGGTCAAGGTGGTC | 140 |
| His324Pro | CTGCCGGGATGGTGACCACCTCGACCACGCTGG | 141 |
| CAT-CCT | | |
| | CCTGGGGCCTCCTGCTCATGATCCTACCTCCGGA | |
| | TGTGCAGCGTGAGCCCATCTGGGAAACAGTGCA | |
| | GGGGCCGAGGGAGGAAGGGTA | |
| | TACCCTTCCTCCCTCGGCCCCTGCACTGTTTCCC | 142 |
| | AGATGGGCTCACGCTGCACATCCGGAGGTAGGA | |
| | TCATGAGCAGGAGGCCCCAGGCCAGCGTGGTCG | |
| | AGGTGGTCACCATCCCGGCAG | |
| | GATCCTACCTCCGGATG | 143 |
| | CATCCGGAGGTAGGATC | 144 |
| Pro325Leu | CCGGGATGGTGACCACCTCGACCACGCTGGCCT | 145 |
| CCG-CTG | | |
| | GGGGCCTCCTGCTCATGATCCTACATCTGGATGT | |
| | GCAGCGTGAGCCCATCTGGGAAACAGTGCAGGG | |
| | GCCGAGGGAGGAAGGGTACAG | |
| | CTGTACCCTTCCTCCCTCGGCCCCTGCACTGTTT | 146 |
| | CCCAGATGGGCTCACGCTGCACATCCAGATGTAG | |
| | GATCATGAGCAGGAGGCCCCAGGCCAGCGTGGT | |
| | CGAGGTGGTCACCATCCCGG | |
| | CCTACATCTGGATGTGC | 147 |
| | GCACATCCAGATGTAGG | 148 |
| Val338Met | TGCTGACCCATTGTGGGGACGCATGTCTGTCCAG | 149 |
| GTG-ATG | | |
| | GCCGTGTCCAACAGGAGATCGACGACATGATAG | |
| | GGCAGGTGCGGCGACCAGAGATGGGTGACCAG | |
| | GCTCACATGCCCTACACCACTG | |
| | CAGTGGTGTAGGGCATGTGAGCCTGGTCACCCAT | 150 |
| | CTCTGGTCGCCGCACCTGCCCTATCATGTCGTCG | |
| | ATCTCCTGTTGGACACGGCCTGGACAGACATGCG | |
| | TCCCCACAATGGGTCAGCA | |
| | TCGACGACATGATAGGG | 151 |
| | CCCTATCATGTCGTCGA | 152 |
| Arg343Gly | GGGACGCATGTCTGTCCAGGCCGTGTCCAACAG | 153 |
| CGG-GGG | | |
| | GAGATCGACGACGTGATAGGGCAGGTGGGGCGA | |
| | CCAGAGATGGGTGACCAGGCTCACATGCCCTACA | |
| | CCACTGCCGTGATTCATGAGG | |
| | CCTCATGAATCACGGCAGTGGTGTAGGGCATGTG | 154 |
| | AGCCTGGTCACCCATCTCTGGTCGCCCCACCTGC | |
| | CCTATCACGTCGTCGATCTCCTGTTGGACACGGC | |
| | CTGGACAGACATGCGTCCC | |
| | GGCAGGTGGGGCGACCA | 155 |
| | TGGTCGCCCCACCTGCC | 156 |
| Arg365His | CAGAGATGGGTGACCAGGCTCACATGCCCTACAC | 157 |
| CGC-CAC | | |
| | CACTGCCGTGATTCATGAGGTGCAGCACTTTGGG | |
| | GACATCGTCCCCCTGGGTGTGACCCATATGACAT | |
| | CCCGTGACATCGAAGTACA | |
| | TGTACTTCGATGTCACGGGATGTCATATGGGTCA | 158 |
| | CACCCAGGGGGACGATGTCCCCAAAGTGCTGCA | |
| | CCTCATGAATCACGGCAGTGGTGTAGGGCATGTG | |
| | AGCCTGGTCACCCATCTCTG | |
| | GGTGCAGCACTTTGGGG | 159 |
| | CCCCAAAGTGCTGCACC | 160 |
| Ile369Thr | ACCAGGCTCACATGCCCTACACCACTGCCGTGAT | 161 |
| ATC-ACC | | |
| | TCATGAGGTGCAGCGCTTTGGGGACACCGTCCC | |
| | CCTGGGTGTGACCCATATGACATCCCGTGACATC | |
| | GAAGTACAGGGCTTCCGCAT | |
| | ATGCGGAAGCCCTGTACTTCGATGTCACGGGATG | 162 |
| | TCATATGGGTCACACCCAGGGGGACGGTGTCCC | |
| | CAAAGCGCTGCACCTCATGAATCACGGCAGTGGT | |
| | GTAGGGCATGTGAGCCTGGT | |
| | TGGGGACACCGTCCCCC | 163 |
| | GGGGGACGGTGTCCCCA | 164 |
| Gly373Ser | ATGCCCTACACCACTGCCGTGATTCATGAGGTGC | 165 |
| GGT-AGT | | |
| | AGCGCTTTGGGGACATCGTCCCCCTGAGTGTGA | |
| | CCCATATGACATCCCGTGACATCGAAGTACAGGG | |
| | CTTCCGCATCCCTAAGGTAG | |
| | CTACCTTAGGGATGCGGAAGCCCTGTACTTCGAT | 166 |
| | GTCACGGGATGTCATATGGGTCACACTCAGGGG | |
| | GACGATGTCCCCAAAGCGCTGCACCTCATGAATC | |
| | ACGGCAGTGGTGTAGGGCAT | |
| | TCCCCCTGAGTGTGACC | 167 |
| | GGTCACACTCAGGGGGA | 168 |
| Val374Met | CCCTACACCACTGCCGTGATTCATGAGGTGCAGC | 169 |
| GTG-ATG | | |
| | GCTTTGGGGACATCGTCCCCCTGGGTATGACCCA | |
| | TATGACATCCCGTGACATCGAAGTACAGGGCTTC | |
| | CGCATCCCTAAGGTAGGCC | |
| | GGCCTACCTTAGGGATGCGGAAGCCCTGTACTTC | 170 |
| | GATGTCACGGGATGTCATATGGGTCATACCCAGG | |
| | GGGACGATGTCCCCAAAGCGCTGCACCTCATGAA | |
| | TCACGGCAGTGGTGTAGGG | |
| | CCCTGGGTATGACCCAT | 171 |
| | ATGGGTCATACCCAGGG | 172 |
| Glu410Lys | GCCCAGGGAACGACACTCATCACCAACCTGTCAT | 173 |
| GAG-AAG | | |
| | CGGTGCTGAAGGATGAGGCCGTCTGGAAGAAGC | |
| | CCTTCCGCTTCCACCCCGAACACTTCCTGGATGC | |
| | CCAGGGCCACTTTGTGAAGC | |
| | GCTTCACAAAGTGGCCCTGGGCATCCAGGAAGT | 174 |
| | GTTCGGGGTGGAAGCGGAAGGGCTTCTTCCAGA | |
| | CGGCCTCATCCTTCAGCACCGATGACAGGTTGGT | |
| | GATGAGTGTCGTTCCCTGGGC | |
| | CCGTCTGGAAGAAGCCC | 175 |
| | GGGCTTCTTCCAGACGG | 176 |
| Glu418Gln | AACCTGTCATCGGTGCTGAAGGATGAGGCCGTCT | 177 |
| GAA-CAA | | |
| | GGGAGAAGCCCTTCCGCTTCCACCCCCAACACTT | |
| | CCTGGATGCCCAGGGCCACTTTGTGAAGCCGGA | |
| | GGCCTTCCTGCCTTTCTCAG | |
| | CTGAGAAAGGCAGGAAGGCCTCCGGCTTCACAA | 178 |
| | AGTGGCCCTGGGCATCCAGGAAGTGTTGGGGGT | |
| | GGAAGCGGAAGGGCTTCTCCCAGACGGCCTCAT | |
| | CCTTCAGCACCGATGACAGGTT | |
| | TCCACCCCCAACACTTC | 179 |
| | GAAGTGTTGGGGGTGGA | 180 |
| Leu421Pro | CGGTGCTGAAGGATGAGGCCGTCTGGGAGAAGC | 181 |
| CTG-CCG | | |
| | CCTTCCGCTTCCACCCCGAACACTTCCCGGATGC | |
| | CCAGGGCCACTTTGTGAAGCCGGAGGCCTTCCT | |
| | GCCTTTCTCAGCAGGTGCCTG | |
| | CAGGCACCTGCTGAGAAAGGCAGGAAGGCCTCC | 182 |
| | GGCTTCACAAAGTGGCCCTGGGCATCCGGGAAG | |
| | TGTTCGGGGTGGAAGCGGAAGGGCTTCTCCCAG | |
| | ACGGCCTCATCCTTCAGCACCG | |
| | ACACTTCCCGGATGCCC | 183 |
| | GGGCATCCGGGAAGTGT | 184 |
| Arg440His | TCTTGCAGGGGTATCACCCAGGAGCCAGGCTCA | 185 |
| CGC-CAC | | |
| | CTGACGCCCCTCCCCTCCCCACAGGCCACCGTG | |
| | CATGCCTCGGGGAGCCCCTGGCCCGCATGGAGC | |
| | TCTTCCTCTTCTTCACCTCCCT | |
| | AGGGAGGTGAAGAAGAGGAAGAGCTCCATGCGG | 186 |
| | GCCAGGGGCTCCCCGAGGCATGCACGGTGGCCT | |
| | GTGGGGAGGGGAGGGGCGTCAGTGAGCCTGGC | |
| | TCCTGGGTGATACCCCTGCAAGA | |
| | CACAGGCCACCGTGCAT | 187 |
| | ATGCACGGTGGCCTGTG | 188 |
| Met451Ile | TGACGCCCCTCCCCTCCCCACAGGCCGCCGTGC | 189 |
| ATG-ATA | | |
| | ATGCCTCGGGGAGCCCCTGGCCCGCATAGAGCT | |
| | CTTCCTCTTCTTCACCTCCCTGCTGCAGCACTTCA | |
| | GCTTCTCGGTGCCCACTGGA | |
| | TCCAGTGGGCACCGAGAAGCTGAAGTGCTGCAG | 190 |
| | CAGGGAGGTGAAGAAGAGGAAGAGCTCTATGCG | |
| | GGCCAGGGGCTCCCCGAGGCATGCACGGCGGC | |
| | CTGTGGGGAGGGGAGGGGCGTCA | |
| | GCCCGCATAGAGCTCTT | 191 |
| | AAGAGCTCTATGCGGGC | 192 |
| Ser486Thr | TCTCGGTGCCCACTGGACAGCCCCGGCCCAGCC | 193 |
| AGC-ACC | | |
| | ACCATGGTGTCTTTGCTTTCCTGGTGACCCCATC | |
| | CCCCTATGAGCTTTGTGCTGTGCCCCGCTAGAAT | |
| | GGGGTACCTAGTCCCCAGCC | |
| | GGCTGGGGACTAGGTACCCCATTCTAGCGGGGC | 194 |
| | ACAGCACAAAGCTCATAGGGGGATGGGGTCACC | |
| | AGGAAAGCAAAGACACCATGGTGGCTGGGCCGG | |
| | GGCTGTCCAGTGGGCACCGAGA | |
| | CCTGGTGACCCCATCCC | 195 |
| | GGGATGGGGTCACCAGG | 196 |

Aliquots of the coisogenic cell collection are thereafter separately contacted with a variety of chemotherapeutic agents presently used for, or contemplated for use in, treatment of breast adenocarcinoma, and alleles that increase or decrease sensitivity to the cytotoxic effects of the agents are identified.

All patents, patent publications, and other published references mentioned herein are hereby incorporated by reference in their entireties as if each had been individually and specifically incorporated by reference herein. While preferred illustrative embodiments of the present invention are described, one skilled in the art will appreciate that the present invention can be practiced by other than the described embodiments, which are presented for purposes of illustration only and not by way of limitation. The present invention is limited only by the claims that follow.

What is claimed is:

1. A collection of cultured cells, comprising:
at least 5 genotypically distinct cells,
wherein each of said at least 5 genotypically distinct cells is coisogenic with respect to the others of said at least 5 genotypically distinct cells at a target locus common thereamong, and
wherein each of said at least 5 genotypically distinct cells can be separately assayed.

2. The cell collection of claim 1, comprising at least 10 genotypically distinct cells.

3. The cell collection of claim 2, comprising at least 25 genotypically distinct cells.

4. The cell population of claim 1, wherein said cells are mammalian cells.

5. The cell population of claim 4, wherein said mammalian cells are human cells.

6. The cell population of claim 4, wherein said mammalian cells are rodent cells.

7. The cell population of claim 6, wherein said rodent cells are mouse cells.

8. The cell population of claim 1, wherein said cells are yeast cells.

9. The cell population of claim 1, wherein said cells are plant cells.

10. The cell collection of claim 1, wherein each of said genotypically distinct cells is disposed in fluid noncommunication with each of the other of said genotypically distinct cells.

11. The cell collection of claim 10, wherein each of said genotypically distinct cells is spatially addressable.

12. The cell collection of claim 1, wherein said genotypically distinct cells collectively include each of the 20 natural amino acids at a single residue encoded at the target locus.

13. The cell collection of claim 1, wherein said genotypically distinct cells collectively include a predetermined amino acid at each residue encoded after the initiator methionine at the target locus.

14. The cell collection of claim 1, wherein said genotypically distinct cells collectively include at least one naturally occurring allele of the target locus.

15. The cell collection of claim 14, wherein said genotypically distinct cells collectively include a plurality of naturally occurring alleles of the target locus.

16. The cell collection of claim 1, wherein said genotypically distinct cells further comprise a common selectable marker at a genomic locus different from said target locus.

17. The cell collection of claim 1, wherein said genotypically distinct cells each further comprises a marker unique to said genotypically distinct cell, said marker being at a locus different from said target locus.

18. The cell collection of claim 1, wherein said target locus is selected from the group consisting of: CYP1A2, CYP2C17, CYP2D6, CYP2E, CYP3A4, CYP4A11, CYP1B1, CYP1A1, CYP2A6, CYP2A13, CYP2B6, CYP2C8, CYP2C9, CYP11A, CYP2C19, CYP2F1, CYP2J2, CYP3A5, CYP3A7, CYP4B1, CYP4F2, CYP4F3, CYP6Dl, CYP6F1, CYP7A1, CYP8, CYP11A, CYP11B1, CYP11B2, CYP17, CYP19, CYP21A2, CYP24, CYP27A1, CYP51, ABCB1, ABCB4, ABCC1, ABCC2, ABCC3, ABCC4, ABCC5, ABCC6, MRP7, ABCC8, ABCC9, ABCC10, ABCC11, ABCC12, EPHX1, EPHX2, LTA4H, TRAG3, GUSB, TMPT, BCRP, HERG, hKCNE2, UDP glucuronosyl transferase (UGT), sulfotransferase, sulfatase, glutathione S-transferase (GST)-alpha, glutathione S-transferase-mu, glutathione S-transferase-pi, ACE, and KCHN2.

19. The cell collection of claim 18, wherein said target locus is ABCB1.

20. The cell collection of any one of claims 1-19, wherein said coisogenic cells are legacy-free.

21. The cell collection of claim 1, wherein said coisogenic cells are exceptionally coisogenic.

22. The cell collection of claim 1, wherein said coisogenic cells are perfectly coisogenic.

23. A kit, comprising:
at least five genotypically distinct cells, said cells contained within separate, structurally discrete, fluidly noncommunicating containers, wherein each of said at least 5 genotypically distinct cells is coisogenic with respect the others of said at least 5 genotypically distinct cells at a target locus common thereamong;
wherein said at least five structurally discrete containers are commonly packaged.

24. The kit of claim 23, wherein said at least five genotypically distinct, commonly packaged, cells constitute a coisogenic cell collection according to claim 1.

25. The kit of claim 23, further comprising:
a computer readable medium, said computer readable medium containing a dataset that describes the target locus genotype of each of said genotypically distinct cells.

26. A method of making a coisogenic cell collection, the method comprising:
collecting at least 5 genotypically distinct cells, each of said genotypically distinct cells being coisogenic with respect to the others of said at least 5 genotypically distinct cells at a target locus common thereamong, into a collection in which each of said at least 5 genotypically distinct cells can be separately assayed.

27. The method of claim 26, further comprising the antecedent step of:
engineering, into at least four of said at least five cultured cells, said cells having derived from a common eukaryotic ancestor cell, a genomic sequence alteration at a target locus common thereamong, said sequence alterations being sufficient to cause at least five distinct protein sequences collectively to be encoded by said cells at said target locus.

28. The method of claim 27, wherein said engineering is effected by introducing a targeting oligonucleotide into each of said at least four cultured cells.

29. The method of claim 27, wherein said engineering step is effected by introducing into each of said at least four cultured cells a recombination-competent substrate into which said genomic sequence alteration has previously been introduced using a targeting oligonucleotide.

30. A kit, comprising:
at least four targeting oligonucleotides of distinct sequence; and
a eukaryotic cell, wherein said oligonucleotides are sufficient for use in the method of claim 28 to create the cell collections of claim 1 from said eukaryotic cell.

31. A method of identifying genotypes of a target locus that alter a cellular phenotype, comprising:
assaying each genotypically distinct cell of a coisogenic cell collection for a common phenotypic characteristic, wherein said genotypically distinct cells are coisogenic at said target locus, and wherein said collection is a coisogenic cell collection according to claim 1;
identifying from said assay results at least one cell having an altered phenotypic characteristic; and
correlating, for at least said at least one cell with altered phenotypic characteristic, the results of said phenotypic assay with said cell's target locus genotype,
the correlation of phenotypic assay results with target locus genotype identifying genotypes of said target locus that alter said cellular phenotype.

32. The method of claim 31, wherein said phenotypic characteristic is responsiveness of said cell to a xenobiotic.

33. The method of claim 31, further comprising the antecedent step of:
contacting said coisogenic cell collection with a xenobiotic.

34. The method of claim 31, wherein said target locus is selected from the group consisting of: CYP1A2, CYP2C17, CYP2D6, CYP2E, CYP3A4, CYP4A11, CYP1B1, CYP1A1, CYP2A6, CYP2A13, CYP2B6, CYP2C8, CYP2C9, CYP11A, CYP2C19, CYP2F1, CYP2J2, CYP3A5, CYP3A7, CYP4B1, CYP4F2, CYP4F3, CYP6D1, CYP6F1, CYP7A1, CYP8, CYP11A, CYP11B1, CYP11B2, CYP17, CYP19, CYP21A2, CYP24, CYP27A1, CYP51, ABCB1, ABCB4, ABCC1, ABCC2, ABCC3, ABCC4, ABCC5, ABCC6, MRP7, ABCC8, ABCC9, ABCC10, ABCC11, ABCC12, EPHX1, EPHX2, LTA4H, TRAG3, GUSB, TMPT, BCRP, HERG, hKCNE2, UDP glucuronosyl transferase (UGT), sulfotransferase, sulfatase, glutathione S-transferase (GST)-alpha, glutathione S-transferase-mu, glutathione S-transferase-pi, ACE, and KCHN2.

35. The method of claim 31, further comprising the step, after said correlating, of:
collecting said correlations into at least one dataset.

36. The method of claim 34, wherein said dataset is recorded on a computer-readable medium.

37. A method of predicting a phenotypic characteristic of a cell based upon its genotype at a target locus, comprising:
using said cell's genotype at said target locus, or a unique identifier thereof, as a query to retrieve from a dataset data that report a correlated phenotypic characteristic,
wherein said dataset includes correlations of a phenotypic characteristic with target locus genotype for at least five cells that are coisogenic at said target locus,
said retrieved phenotypic characteristic providing a prediction of said cell's phenotypic characteristic.

38. The method of claim 37, wherein said at least five cells that are coisogenic at said target locus genotype are a cell collection according to claim 1.

39. The method of claim 37, wherein said target locus is selected from the group consisting of: CYP1A2, CYP2C17, CYP2D6, CYP2E, CYP3A4, CYP4A11, CYP1B1, CYP1A1, CYP2A6, CYP2A13, CYP2B6, CYP2C8, CYP2C9, CYP11A, CYP2C19, CYP2F1, CYP2J2, CYP3A5, CYP3A7, CYP4B1, CYP4F2, CYP4F3, CYP6D1, CYP6F1, CYP7A1, CYP8, CYP11A, CYP11B1, CYP11B2, CYP17, CYP19, CYP21A2, CYP24, CYP27A1, CYP51, ABCB1, ABCB4, ABCC1, ABCC2, ABCC3, ABCC4, ABCC5, ABCC6, MRP7, ABCC8, ABCC9, ABCC10, ABCC11, ABCC12, EPHX1, EPHX2, LTA4H, TRAG3, GUSB, TMPT, BCRP, HERG, hKCNE2, UDP glucuronosyl transferase (UGT), sulfotransferase, sulfatase, glutathione S-transferase (GST)-alpha, glutathione S-transferase-mu, glutathione S-transferase-pi, ACE, and KCHN2.
